SEQUENCE LISTING (14 sequences)

SEQ ID NO: 1 - protein, 441 aa, Homo sapiens
MGRKKIQITRIMDERNRQVTFTKRKFGLMKKAYELSVLCDCEIALIIFNSTNKLFQYASTDMDKVLLKYTEYNEPHESRTNSDIVETLRKKGLNGCDSPDPDADDSVGHSPESEDKYRKINEDIDLMISRQRLCAVPPPNFEMPVSIPVSSHNSLVYSNPVSSLGNPNLLPLAHPSLQRNSMSPGVTHRPPSAGNTGGLMGGDLTSGAGTSAGNGYGNPRNSPGLLVSPGNLNKNMQAKSPPPMNLGMNNRKPDLRVLIPPGSKNTMPSVSEDVDLLLNQRINNSQSAQSLATPVVSVATPTLPGQGMGGYPSAISTTYGTEYSLSSADLSSLSGFNTASALHLGSVTGWQQQHLHNMPPSALSQLGDRTTTPSRYPQHTRHEAGRSPVDSLSSCSSSYDGSDREDHRNEFHSPIGLTRPSPDERESPSVKRMRLSEGWAT

SEQ ID NO: 2 - DNA, 1326 bp, Homo sapiens
  source 1..1326: mol_type = genomic DNA
atggggagaaaaaagattcagattacgaggattatggatgaacgtaacagacaggtgacatttacaaagaggaaatttgggttgatgaagaaggcttatgagctgagcgtgctgtgtgactgtgagattgcgctgatcatcttcaacagcaccaacaagctgttccagtatgccagcaccgacatggacaaagtgcttctcaagtacacggagtacaacgagccgcatgagagccggacaaactcagacatcgtggagacgttgagaaagaagggccttaatggctgtgacagcccagaccccgatgcggacgattccgtaggtcacagccctgagtctgaggacaagtacaggaaaattaacgaagatattgatctaatgatcagcaggcaaagattgtgtgctgttccacctcccaacttcgagatgccagtctccatcccagtgtccagccacaacagtttggtgtacagcaaccctgtcagctcactgggaaaccccaacctattgccactggctcacccttctctgcagaggaatagtatgtctcctggtgtaacacatcgacctccaagtgcaggtaacacaggtggtctgatgggtggagacctcacgtctggtgcaggcaccagtgcagggaacgggtatggcaatccccgaaactcaccaggtctgctggtctcacctggtaacttgaacaagaatatgcaagcaaaatctcctcccccaatgaatttaggaatgaataaccgtaaaccagatctccgagttcttattccaccaggcagcaagaatacgatgccatcagtgtctgaggatgtcgacctgcttttgaatcaaaggataaataactcccagtcggctcagtcattggctaccccagtggtttccgtagcaactcctactttaccaggacaaggaatgggaggatatccatcagccatttcaacaacatatggtaccgagtactctctgagtagtgcagacctgtcatctctgtctgggtttaacaccgccagcgctcttcaccttggttcagtaactggctggcaacagcaacacctacataacatgccaccatctgccctcagtcagttgggagaccgtaccaccaccccttcgagatacccacaacacacgcgccacgaggcggggagatctcctgttgacagcttgagcagctgtagcagttcgtacgacgggagcgaccgagaggatcaccggaacgaattccactcccccattggactcaccagaccttcgccggacgaaagggaaagtccctcagtcaagcgcatgcgactttctgaaggatgggcaacatga

SEQ ID NO: 3 - DNA, 1326 bp, synthetic construct
  source 1..1326: mol_type = other DNA
atgggaagaaagaaaatccaaataaccagaatcatggacgagcggaataggcaagttaccttcacgaaacggaagttcggactgatgaagaaggcctacgaactcagtgtcttgtgcgattgcgagatcgcccttattatctttaattctacaaacaaactcttccaatacgcttccactgatatggataaggtgttgctgaagtacaccgagtataatgaaccacacgaaagtcgtaccaattctgatattgtagaaaccctcaggaagaaaggactcaacgggtgcgatagtcctgatccggacgctgatgactctgttgggcattctcccgaatcagaagataaatatagaaagatcaatgaggacatcgacctgatgatttcccggcagaggctctgcgcggtcccgccccctaattttgaaatgcccgtgagtattcctgtctctagtcataatagcctggtctattccaatccagtgtcaagccttgggaatccaaatctgctgcctctcgcccatccctcattgcaacggaactcaatgagtcccggcgtcacccacagaccgccatccgccggaaatactggcggccttatgggcggtgatctgacatcaggcgctgggacttcagccggaaatggatacggaaacccacgcaatagtccgggcttgcttgtgtctccaggaaatcttaataagaacatgcaggctaagagtccaccacctatgaacctgggcatgaacaataggaagcccgacttgagggtgctcatcccacccggttctaagaacaccatgccttccgtttcagaagacgtggatctcttgctcaaccagcgaatcaacaattctcaaagcgcccaatctcttgcaacgcctgtagtctctgttgctacacccacactgcctgggcaggggatgggcggctaccctagcgctatctccactacgtacggcactgaatattcccttagcagcgccgatctcagcagtttgagcggcttcaatacagcttccgcgctgcatctggggtctgttactggttggcagcaacagcatttgcacaatatgccgcctagcgcgcttagccaactcggggatcggactacaacaccctctcgctatccgcagcatactcggcatgaagcaggacgatcccccgtggattcactgtctagttgctcctcttcctatgatggcagtgatagggaagaccatcgtaatgagtttcatagtccaatcggcctgacacggccatcacctgatgagcgcgagtctccttctgtgaaacggatgcggctgtcagaggggtgggctacctaa

SEQ ID NO: 4 - protein, 521 aa, Homo sapiens
MGRKKIQIQRITDERNRQVTFTKRKFGLMKKAYELSVLCDCEIALIIFNHSNKLFQYASTDMDKVLLKYTEYNEPHESRTNADIIETLRKKGFNGCDSPEPDGEDSLEQSPLLEDKYRRASEELDGLFRRYGSTVPAPNFAMPVTVPVSNQSSLQFSNPSGSLVTPSLVTSSLTDPRLLSPQQPALQRNSVSPGLPQRPASAGAMLGGDLNSANGACPSPVGNGYVSARASPGLLPVANGNSLNKVIPAKSPPPPTHSTQLGAPSRKPDLRVITSQAGKGLMHHLTEDHLDLNNAQRLGVSQSTHSLTTPVVSVATPSLLSQGLPFSSMPTAYNTDYQLTSAELSSLPAFSSPGGLSLGNVTAWQQPQQPQQPQQPQPPQQQPPQPQQPQPQQPQQPQQPPQQQSHLVPVSLSNLIPGSPLPHVGAALTVTTHPHISIKSEPVSPSRERSPAPPPPAVFPAARPEPGDGLSSPAGGSYETGDRDDGRGDFGPTLGLLRPAPEPEAEGSAVKRMRLDTWTLK

SEQ ID NO: 5 - DNA, 1566 bp, Homo sapiens
  source 1..1566: mol_type = genomic DNA
atggggaggaaaaagattcagatccagcgaatcaccgacgagcggaaccgacaggtgactttcaccaagcggaagtttggcctgatgaagaaggcgtatgagctgagcgtgctatgtgactgcgagatcgcactcatcatcttcaaccactccaacaagctgttccagtacgccagcaccgacatggacaaggtgctgctcaagtacacggagtacaatgagccacacgagagccgcaccaacgccgacatcatcgagaccctgaggaagaagggcttcaacggctgcgacagccccgagcccgacggggaggactcgctggaacagagccccctgctggaggacaagtaccgacgcgccagcgaggagctcgacgggctcttccggcgctatgggtcaactgtcccggcccccaactttgccatgcctgtcacggtgcccgtgtccaatcagagctcactgcagttcagcaatcccagcggctccctggtcaccccttccctggtgacatcatccctcacggacccgcggctcctgtccccccagcagccagcactacagaggaacagtgtgtctcctggcctgccccagcggccagctagtgcgggggccatgctggggggtgacctgaacagtgctaacggagcctgccccagccctgttgggaatggctacgtcagtgctcgggcttcccctggcctcctccctgtggccaatggcaacagcctaaacaaggtcatccctgccaagtctccacccccacctacccacagcacccagcttggagcccccagccgcaagcccgacctgcgagtcatcacttcccaggcaggaaaggggttaatgcatcacttgactgaggaccatttagatctgaacaatgcccagcgccttggggtctcccagtctactcattcgctcaccaccccagtggtttctgtggcaacgccgagtttactcagccagggcctccccttctcttccatgcccactgcctacaacacagattaccagttgaccagtgcagagctctcctccttaccagcctttagttcacctggggggctgtcgctaggcaatgtcactgcctggcaacagccacagcagccccagcagccgcagcagccacagcctccacagcagcagccaccgcagccacagcagccacagccacagcagcctcagcagccgcaacagccacctcagcaacagtcccacctggtccctgtatctctcagcaacctcatcccgggcagccccctgccccacgtgggtgctgccctcacagtcaccacccacccccacatcagcatcaagtcagaaccggtgtccccaagccgtgagcgcagccctgcgcctccccctccagctgtgttcccagctgcccgccctgagcctggcgatggtctcagcagcccagccgggggatcctatgagacgggagaccgggatgacggacggggggacttcgggcccacactgggcctgctgcgcccagccccagagcctgaggctgagggctcagctgtgaagaggatgcggcttgatacctggacattaaagtga

SEQ ID NO: 6 - DNA, 1566 bp, synthetic construct
  source 1..1566: mol_type = other DNA
atgggaagaaagaaaatccaaatacaaagaattacagatgaacgcaacagacaagttacattcaccaaaaggaagttcggtttgatgaagaaagcctacgaactttcagtcctctgcgattgtgaaatagccctgattatcttcaatcatagcaacaaactgttccaatatgccagcacagatatggataaagtactcctgaagtatacagaatataacgaaccccacgaatcacggacaaatgctgatattattgaaacgcttcgcaagaaagggtttaatgggtgtgatagtcctgaaccagatggcgaagatagcttggagcaatccccattgctcgaagataaatatcgcagagcttctgaagaattggatggactgtttagacggtacggctccaccgtgcctgctccaaatttcgctatgccagttacagttccagtttctaaccaatcaagcctgcaattttctaacccttctggtagtctcgtgacaccctctctcgttacgtctagcttgacagatcctaggctgttgtctcctcaacaacctgccctgcaacggaattcagtctcaccgggactcccacaacgtcccgcaagcgcaggagcgatgctcggcggggatcttaatagcgcgaatggcgcttgtccttcacccgtaggcaacgggtatgtgtcagcgcgcgcaagccccggactgctgcccgttgcgaacggtaattcactgaacaaagtgattcccgcaaagagtcctcctccaccaacacattctacacaactgggcgcacctagtagaaaacctgatttgcgcgtgattacctctcaagccggcaaaggcctgatgcaccatctcacggaagatcaccttgacctcaataacgcacaaagactcggagtgagccaatccacacactccctgacgacgcccgtcgtctccgtagccaccccatcccttttgtcacaaggtctgccatttagctctatgcctacagcttataatactgactatcaactgacgtccgctgaactgagttcacttcctgctttctccagccccggtggcctctcacttgggaacgtgaccgcgtggcagcaacctcaacaaccacaacaaccccaacaaccgcaaccgcctcaacaacaaccccctcaaccgcaacaaccccaaccccaacaaccgcaacaaccccagcaaccgccccaacagcaaagccatcttgttccagtgagtctgtccaatctgattcctggaagcccactccctcatgttggcgcggctctgactgtgactactcatcctcatatttctatcaaaagtgagcctgtttctccctctagagaaagatctcccgctccgcccccacccgccgtctttcctgccgctagacccgaaccaggggacgggctgtcctcacctgcaggcggaagttacgaaaccggggatagagacgatggtagaggtgattttggccctactcttggactgctccgacctgcaccggaacccgaagccgaaggaagcgccgtaaaacgaatgagactggacacatggacgcttaaataa

SEQ ID NO: 7 - protein, 463 aa, Homo sapiens
MYGNYSHFMKFPAGYGGSPGHTGSTSMSPSAALSTGKPMDSHPSYTDTPVSAPRTLSAVGTPLNALGSPYRVITSAMGPPSGALAAPPGINLVAPPSSQLNVVNSVSSSEDIKPLPGLPGIGNMNYPSTSPGSLVKHICAICGDRSSGKHYGVYSCEGCKGFFKRTIRKDLIYTCRDNKDCLIDKRQRNRCQYCRYQKCLVMGMKREAVQEERQRSRERAESEAECATSGHEDMPVERILEAELAVEPKTESYGDMNMENSTNDPVTNICHAADKQLFTLVEWAKRIPHFSDLTLEDQVILLRAGWNELLIASFSHRSVSVQDGILLATGLHVHRSSAHSAGVGSIFDRVLTELVSKMKDMQMDKSELGCLRAIVLFNPDAKGLSNPSEVETLREKVYATLEAYTKQKYPEQPGRFAKLLLRLPALRSIGLKCLEHLFFFKLIGDTPIDTFLMEMLETPLQIT

SEQ ID NO: 8 - DNA, 1392 bp, Homo sapiens
  source 1..1392: mol_type = genomic DNA
atgtatggaaattattctcacttcatgaagtttcccgcaggctatggaggctcccctggccacactggctctacatccatgagcccatcagcagccttgtccacagggaagccaatggacagccaccccagctacacagataccccagtgagtgccccacggactctgagtgcagtggggacccccctcaatgccctgggctctccatatcgagtcatcacctctgccatgggcccaccctcaggagcacttgcagcgcctccaggaatcaacttggttgccccacccagctctcagctaaatgtggtcaacagtgtcagcagttcagaggacatcaagcccttaccagggcttcccgggattggaaacatgaactacccatccaccagccccggatctctggttaaacacatctgtgccatctgtggagacagatcctcaggaaagcactacggggtatacagttgtgaaggctgcaaagggttcttcaagaggacgataaggaaggacctcatctacacgtgtcgggataataaagactgcctcattgacaagcgtcagcgcaaccgctgccagtactgtcgctatcagaagtgccttgtcatgggcatgaagagggaagctgtgcaagaagaaagacagaggagccgagagcgagctgagagtgaggcagaatgtgctaccagtggtcatgaagacatgcctgtggagaggattctagaagctgaacttgctgttgaaccaaagacagaatcctatggtgacatgaatatggagaactcgacaaatgaccctgttaccaacatatgtcatgctgctgacaagcagcttttcaccctcgttgaatgggccaagcgtattccccacttctctgacctcaccttggaggaccaggtcattttgcttcgggcagggtggaatgaattgctgattgcctctttctcccaccgctcagtttccgtgcaggatggcatccttctggccacgggtttacatgtccaccggagcagtgcccacagtgctggggtcggctccatctttgacagagtcctaactgagctggtttccaaaatgaaagacatgcagatggacaagtcggaactgggatgcctgcgagccattgtactctttaacccagatgccaagggcctgtccaacccctctgaggtggagactctgcgagagaaggtttatgccacccttgaggcctacaccaagcagaagtatccggaacagccaggcaggtttgccaagctgctgctgcgcctcccagctctgcgttccattggcttgaaatgcctggagcacctcttcttcttcaagctcatcggggacacccccattgacaccttcctcatggagatgttggagaccccgctgcagatcacctga

SEQ ID NO: 9 - DNA, 1392 bp, synthetic construct
  source 1..1392: mol_type = other DNA
atgtacggcaactactcacattttatgaaattcccagccggatacggcgggtctcccggccatacaggtagtacgagcatgtcccccagcgcggcgctgagcaccggcaaacccatggattcccatccttcttataccgacacgcctgtcagcgctcccagaacactctctgccgtcggcacgcctctgaacgctcttggtagtccctacagagttattacatccgctatgggaccaccgagtggggcgctggctgccccacccggcataaatctcgtggctccgccaagctcccaattgaacgtcgtgaattctgtatcttctagtgaagacataaaacctcttcctggcctgccgggtatcggtaatatgaattatccttctacgtcccctggttcactcgtgaagcatatttgcgctatttgcggcgatcggtcatctgggaaacattatggagtctattcctgcgagggatgtaagggtttctttaaacgtacaatccgtaaagatcttatttatacctgcagggacaacaaggattgtcttatcgacaaaaggcaacgaaatcggtgtcaatattgcagataccagaaatgtctcgtgatgggtatgaaaagagaggccgtccaggaagagcgccaacggagtagagaaagggcggaatccgaagctgagtgcgccactagcgggcacgaggatatgcccgtcgaacgtatccttgaggcagagctggcggtcgagcccaaaactgagagctacggggatatgaacatggaaaattccactaacgatccagtcactaatatctgccacgcagccgataaacaactgtttacattggtcgagtgggctaaacgcataccacattttagtgatctgacacttgaagatcaagttatccttctccgagctggctggaacgagcttctcatagcatccttttctcatagatctgtgtctgtccaagacgggattctcctcgcaacaggcctgcacgtgcataggtccagcgcgcatagcgcaggcgtggggagtattttcgatagggtgttgaccgaactcgtgagcaagatgaaggatatgcaaatggataaatcagagttgggctgtctccgcgcaatagtgttgttcaatcccgacgctaaagggctcagcaatcctagtgaagtcgaaaccttgagggaaaaggtatacgctacattggaagcgtataccaaacaaaagtaccccgagcaacctgggcgattcgcaaaacttcttttgcggctgcctgccctgcggagtatcgggcttaagtgtttggaacatctgttcttctttaaactgataggtgatactccgatcgacacatttctgatggaaatgctcgaaacgcctctccaaattacataa

SEQ ID NO: 10 - protein, 299 aa, Homo sapiens [sequence omitted]

SEQ ID NO: 11 - DNA, 900 bp, Homo sapiens
  source 1..900: mol_type = genomic DNA
atgatggcgtatatgaacccggggccccactattctgtcaacgccttggccctaagtggccccagtgtggatctgatgcaccaggctgtgccctacccaagcgcccccaggaagcagcggcgggagcgcaccaccttcacccggagccaactggaggagctggaggcactgtttgccaagacccagtacccagacgtctatgcccgtgaggaggtggctctgaagatcaatctgcctgagtccagggttcaggtttggttcaagaaccggagggctaaatgcaggcagcagcgacagcagcagaaacagcagcagcagcccccagggggccaggccaaggcccggcctgccaagaggaaggcgggcacgtccccaagaccctccacagatgtgtgtccagaccctctgggcatctcagattcctacagtccccctctgcccggcccctcaggctccccaaccacggcagtggccactgtgtccatctggagcccagcctcagagtcccctttgcctgaggcgcagcgggctgggctggtggcctcagggccgtctctgacctccgccccctatgccatgacctacgccccggcctccgctttctgctcttccccctccgcctatgggtctccgagctcctatttcagcggcctagacccctacctttctcccatggtgccccagctagggggcccggctcttagccccctctctggcccctccgtgggaccttccctggcccagtcccccacctccctatcaggccagagctatggcgcctacagccccgtggatagcttggaattcaaggaccccacgggcacctggaaattcacctacaatcccatggaccctctggactacaaggatcagagtgcctggaagtttcagatcttgtag

SEQ ID NO: 12 - DNA, 900 bp, synthetic construct
  source 1..900: mol_type = other DNA
atgatggcctacatgaatcctggtccacattactcagtaaatgctctcgcacttagcggtcctagcgtagacctcatgcatcaagcggtcccgtatccctcagcgccgcggaaacaaagacgtgaaaggacaacatttactagatctcagcttgaagaactcgaagccttgttcgcgaaaacacaatatcccgatgtgtacgcacgggaagaagtcgccctcaaaataaacttgccagaatctcgtgtacaagtgtggtttaagaatagacgcgccaagtgtagacaacaaagacaacaacaaaagcaacaacaacaaccgccgggcggtcaagctaaagctaggcccgctaaacggaaagctggtacaagcccccggccgtcaactgacgtttgccccgatccacttggtatttctgactcttattccccgcccttgcctggaccgagcggtagtcccacaaccgctgtcgcaacggtatctatttggtccccggccagcgaatctcccctgccagaagcacaacgcgcaggcctcgtcgcatccggcccttcactcactagcgcaccatacgcaatgacgtatgcaccagctagtgcattttgttcctcaccttctgcatacggcagcccctcatcatacttttctgggctggacccttatctgtcacctatggtcccgcaattgggcggacctgccctgagtccacttagcggaccgagcgtcggtccgtcactcgctcaaagcccgacatctctcagcggtcaatcttacggagcatattcacctgtcgactcccttgagtttaaagatccaactgggacatggaagtttacttataacccaatggacccactcgattataaagaccaatccgcgtggaaattccaaattctctga

SEQ ID NO: 13 - DNA, 681 bp, synthetic construct
  source 1..681: mol_type = other DNA
aacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 14 - DNA, 699 bp, synthetic construct
  source 1..699: mol_type = other DNA
ctagtgatctaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggatcc

(19) United States
(12) Patent Application Publication
(10) Pub. No.: US 2025/0333456 A1
(43) Pub. Date: Oct. 30, 2025

(54) CELL CONVERSION

(71) Applicant: Mogrify Limited, Cambridge, Cambridgeshire (GB)

(72) Inventors: Geraint John PARFITT, Cambridge, Cambridgeshire (GB); Katarina JOVANOVIC, Cambridge, Cambridgeshire (GB); Evdokia PAZA, Cambridge, Cambridgeshire (GB); Minkyung SUNG, Cambridge, Cambridgeshire (GB); Kalaivani RAJU, Cambridge, Cambridgeshire (GB); Julian GOUGH, Cambridge, Cambridgeshire (GB); David MATTHEWS, Cambridge, Cambridgeshire (GB); Tina STORM, Cambridge, Cambridgeshire (GB); Regan HAMEL, Cambridge, Cambridgeshire (GB)

(21) Appl. No.: 18/855,287

(22) PCT Filed: Apr. 11, 2023
(86) PCT No.: PCT/GB2023/050953

§ 371 Date: Oct. 8, 2024

(30) Foreign Application Priority Data
Apr. 11, 2022 (GB) 2205265.8

(51) Int. Cl. C07K 14/47; A61K 38/17; A61K 48/00; C12N 15/86

(52) U.S. Cl. CPC C07K 14/4705; A61K 38/1709; A61K 48/0058; C12N 15/86; C12N 2740/15043; C12N 2750/14143; C12N 2830/008

(57) ABSTRACT

The present application relates to a nucleic acid molecule comprising a promoter operably linked to a nucleic acid sequence encoding Myocyte Enhancer Factor 2C (MEF2C), or a functional variant thereof, wherein the promoter is for expression of MEF2C in macroglia. Also provided are vectors, compositions, products, methods, cells, medical uses and methods of treatment.

### TECHNICAL FIELD

The present disclosure relates to methods and compositions for the conversion of source cells (such as Müller Glia cells and astrocytes) to cone photoreceptor cells and/or Retinal Pigment Epithelium (RPE)-like cells by introducing transcription factors, optionally one, two, three, or all of MEF2C, MEF2D, RXRG, and/or CRX, into the source cells, and methods for treating a retinal disease or degeneration.

### BACKGROUND TO THE INVENTION

Photoreceptors are sensory neuronal cells found in all vertebrates and play a crucial role in the detection and transduction of light signals, which is essential for vision. The two photoreceptors are the cones and rods. Of the two types of photoreceptors, rods are activated in low light and cones are activated in bright light of specific wavelengths, according to their expression of the photopigments rhodopsin or S/M/L opsin, respectively. Cones are mostly concentrated in the macula, central region of the retina and are required for central, high acuity vision, and colour perception. Despite their importance in the retina, rod and cone cells cannot be renewed in the event of disease, trauma, or injury. Progressive loss of photoreceptors and vision due to genetic mutation (inherited retinal dystrophies), pathological damage or environmental damage results in retinal degeneration, and activation of Muller glia cells, which typically support the metabolism and nutrition of retinal cell types. In the developed world, conditions such as advanced retinitis pigmentosa (RP), age-related macular degeneration (AMD) and diabetic retinopathy, all characterised by photoreceptor loss, are the main causes of registered blindness. AMD is one of the most common retinal degeneration disorders, predominately affecting adults over the age of 40, and accounting for 8% of all blindness worldwide. There are more than 20,000 cases per year in the UK. It is projected that AMD and other retinal degeneration disorders will increase, given the trend towards an ageing population. AMD is characterised by the loss of cone cells resulting in central and colour vision problems. There is currently a need for treatment of such progressive retinal degeneration diseases. Currently available treatments for the wet form of AMD only slow the progression of the disease by inhibiting angiogenesis but it is inevitable that those diagnosed with AMD will end up with vision loss. With the projection that more of the population will suffer from AMD, it is imperative to identify cures to stop or reverse the loss of cone photoreceptors.

Photoreceptors convert light into electrical signals in the process of visual phototransduction. Phototransduction requires the expression of many genes that uniquely mark photoreceptors and understanding the full cascade of regulatory events that control the development of photoreceptors is an unresolved problem. Most studies to date have attempted to identify transcription factors expressed by retinal progenitors and early photoreceptors over the course of retinal development, and generally have not considered photoreceptor regeneration. Swaroop et al., Nature Reviews Neuroscience; 563-576 (2010) discusses the transcriptional regulation of photoreceptor development. Stating that the balanced actions of six key transcription factors (the paired-type homeodomain transcription factor OTX2, cone-rod homeobox protein (CRX), neural retina leucine zipper protein (NRL), photoreceptor-specific nuclear receptor (NR2E3), nuclear receptor RORB and thyroid hormone receptor β2 (TRB2)) are crucial as retinal progenitors commit to a rod or cone lineage. WO2021/253078 discloses a process to produce rod photoreceptor cells from glial cells by increasing the protein expression of one or more transcription factors selected from ASCL1, NEUROD1, NRL, NR2E3, RAX, RORB, OTX2, CRX and PAX6. Specifically, Müller glia cells are reported to be reprogrammed to induced photoreceptor cells that are positive for rod photoreceptor cell markers.

The retinal pigment epithelium (RPE) is sandwiched between the neuroretina and the choroid, serving multiple roles including metabolic support of the retina, recycling of retinal chromophores, absorption of scattered light, and phagocytosis of shed photoreceptor outer segments. The RPE cells form a cobblestone pigmented monolayer of polarised, highly specialised epithelium cells that are located directly adjacent to the light-sensing photoreceptors (rods and cones). The apical processes enwrap the photoreceptor outer segment (POS), whereas the highly infolded RPE basal membrane is attached to the Bruch's membrane, which separates the RPE from a layer of fenestrated capillaries (the choriocapillaris). The morphological specialisations of the RPE facilitate its multiple functions which include transport of nutrients from the blood to the neural retina, regulation of retinal water transport and ionic composition of the subretinal space to maintain the excitability of the photoreceptors. The pigment-containing melanosomes of the RPE furthermore protect the retinal cells from photo-oxidation and absorb light scatter. The specialised phagocytosis and degradation of the damaged POS tips by the RPE is essential for photoreceptor survival and for maintenance of normal vision. Thus, loss of RPE function may ultimately lead to a secondary loss of the overlying photoreceptors.

Current therapeutic approaches to treating dry AMD are focused on preventing or slowing down disease progression, targeting known cellular pathologies at the earlier stages of the disease, and an increasing number are reaching clinical trial stages. In contrast, efforts to repopulate macular atrophic areas of RPE have focused on cellular regenerative therapies with transplantation of healthy RPE cells derived from a number of stem cell sources into the atrophic macular region. Zhang et al (2014), Protin Cell, 5(1): 48-58 described the direct conversion of human fibroblasts to RPE-like cells by defined factors including PAX6, OTX2, MITF, cMYC, RAX, CRX, LKF4 and NRL, of which OTX2, MITF, cMYC, RAX, CRX are said to be “crucial”. Woogeng et al (2021), Stem Cell Reports, 17, 1-18 describe inducing human RPE-like cells from somatic tissue and report that four TFs (MITF, OTX2, LIN28, MYCL), enhanced by CRX and small molecules, could convert human fibroblasts to bulk cultures containing RPE-like cells. A number of clinical trials for treating dry AMD are ongoing, however, so far only with limited success.

Abnormalities, dysfunction and/or death of photoreceptors constitute the primary cause of visual impairment or blindness in most retinal diseases. Abnormalities, dysfunction and/or death of RPE cells may also be evident in retinal diseases, such as in Geographic Atrophy (GA), a sub-segment of late-stage AMD. Therefore, innovative strategies for therapy aim to replace lost or damaged photoreceptors and/or RPE cells by cell replacement or cell conversion of other cell types towards photoreceptor and/or RPE lineage in regenerative therapies.

### SUMMARY OF THE INVENTION

There remains a need in the art to provide methods for generating photoreceptor and/or RPE cells in vivo, ex vivo and in vitro, as potential therapeutics.

Cell-based regenerative therapies using induced pluripotent stem cells or embryonic stem cells have been extensively studied as a replacement for current treatments. However, cells such as embryonic stem cells, retinal progenitors, and differentiated cones, are difficult to transplant into the retina, due to the intricate wiring between photoreceptors and interneurons. An alternative method is the transdifferentiation or direct cell conversion of a cell type to another cell type without going through an intermediate pluripotent state. However, the main barrier associated with transdifferentiation of cells is the difficulty in identifying the elements such as transcription factors, required for successful transdifferentiation of cells that generate target cells with the phenotypic and functional characteristics of the desired cells. In most instances, the required elements remain unknown. Current methods to identify transcription factors required for the transdifferentiation process often rely on literature mining and trial and error methods. This approach is inefficient and costly to test out plausible sets of factors. The present inventors can efficiently identify transcription factors for converting one cell to another cell using their proprietary technology, known as MOGRIFY (see for example WO2017106932). The present invention relates transcription factors predicted in the MOGRIFY version 2.5 system. In vitro and in vivo regeneration of functional photoreceptors would therefore greatly assist the treatment of retinal diseases and retinal degeneration. Accordingly, there is a need in the art to develop a method that can generate functional cone photoreceptors and/or RPE cells. Advantageously, the present inventors have identified a method that can transdifferentiate Müller glia cells to cone photoreceptors and/or RPE cells in vitro and in vivo using transcription factors predicted by their proprietary technology. This was unexpected as Müller glia have the potential to regenerate photoreceptors in lower order vertebrates, such as zebrafish, however, they have limited regenerative capacity in mammals (Nat Rev Neurosci.2014 July; 15(7); 431-442). To the best of our knowledge, Müller Glia have not been previously shown to regenerate RPE cells, even in lower vertebrates.

A further advantage of using the one or more transcription factors according to the present invention is that they are shown herein to produce Retinal Pigment Epithelium (RPE)-like cells, which may functionally support photoreceptors and may therefore extend the surprising therapeutic potential of the one or more transcription factors according to the present invention. For example, Geographic Atrophy (GA), a sub-segment of late-stage AMD, is characterised by the loss of photoreceptors and retinal pigment epithelial (RPE) cells in the macula. GA affects over 8 million people globally, causing a loss of vision due to degeneration of the macula and subsequent cone cell loss. There is currently no approved treatment for GA.

An additional advantage of using the one or more transcription factors according to the present invention is that they are shown herein to increase levels of brain-derived neurotrophic factor (BDNF). BDNF secretion is an established function of RPE cells. BDNF has been proposed as a therapeutic candidate for neurodegenerative diseases because of its potent neuroprotective effect. This includes a proposed role in treatment of glaucoma based on its protective effects on retinal ganglion cells (RGCs) (Kimura et al, Int. J. Mol. Sci. 2016, 17, 1584). BDNF has also been shown to protect cones against phototoxicity (Valiente-Soriano et al, Transl Vis Sci Technol. 2019 Dec. 16;8(6):36). Without being bound by theory, increased expression of BDNF therefore represents a further potential mechanism by which the one or more transcription factors according to the present invention may provide effective treatments for retinal disease or degeneration.

BDNF is a known pro-survival factor for a variety of neuronal cell types, including Retinal Ganglion Cells (RGCs). The loss of RGCs underlies glaucoma. The administration of BDNF has been proposed as a therapeutic strategy for glaucoma accordingly. However, the direct usefulness of BDNF is limited by its short half life and its inability to cross the blood brain barrier. The data disclosed herein indicated that MEF2C, the combination of MEF2C, MEF2D and RXRG, and to a lesser extent CRX, upregulate BDNF expression in Muller glia, which may be indicative of their transdifferentiation to RPE-like cells. The transcription factors disclosed herein (including the nucleic acid molecules, vectors, compositions, products and cells according to the invention) may be advantageous for use in treatment of glaucoma accordingly.

The present invention therefore provides a therapeutic strategy with surprisingly broad applicability in retinal disease or degeneration, to conditions characterised by loss or dysfunction of RPE cells and/or photoreceptors, particularly cone photoreceptors. The potentially broad applicability is provided by the surprising potential of the at least one or more transcription factors of the invention in replacement of cone-like photoreceptors and/or RPE-like cells with the associated neuroprotective effects of BDNF.

According to a first aspect, the invention provides a nucleic acid molecule comprising a promoter operably linked to a nucleic acid sequence encoding Myocyte Enhancer Factor 2C (MEF2C), or a functional variant thereof, wherein the promoter is for expression of MEF2C in macroglia.

According to a second aspect, the invention provides a vector comprising the nucleic acid molecule according to the first aspect.

According to a third aspect, the invention provides a composition comprising the nucleic acid molecule according to the first aspect or the vector according to the second aspect, and a pharmaceutically acceptable carrier.

According to a fourth aspect, the invention provides a product comprising
(a) a first nucleic acid molecule, according to the first aspect, and
(b) a second nucleic acid molecule, comprising a promoter operably linked to a nucleic acid sequence encoding one or more transcription factor selected from the group consisting of Myocyte Enhancer Factor 2D (MEF2D), Retinoid X Receptor Gamma (RXRG) and Cone-Rod Homeobox (CRX), or functional variants thereof, or any combination thereof, wherein the promoter is for expression of the one or more transcription factor in macroglia;
   as a combined preparation for simultaneous, separate or sequential use in the treatment of retinal disease or degeneration.

According to a fifth aspect, the invention provides a method of converting a retinal source cell to a retinal target cell by introducing one or more transcription factor comprising MEF2C, or a functional variant thereof, into the retinal source cell, thereby converting the retinal source cell into the retinal target cell.

According to a sixth aspect, the invention provides a cell produced by the method of the fifth aspect.

According to a seventh aspect, the invention provides the nucleic acid molecule according to the first aspect, the vector according to the second aspect, the composition according to the third aspect, or the cell according to the sixth aspect for use in the treatment of retinal disease or degeneration.

According to an eighth aspect, the invention provides a method of treating retinal disease or degeneration in a subject comprising administering to a retina of the subject in need thereof a therapeutically effective amount of the nucleic acid molecule according to the first aspect, the vector according to the second aspect, the composition according to the third aspect, the product according to the fourth aspect, or the cell according to the sixth aspect.

Any of the features described herein in respect of any of the above-mentioned aspects of the invention may be combined mutatis mutandis with the other aspects of the invention.

### BRIEF DESCRIPTION OF THE DRAWINGS

FIG. 1. Reprogramming of Müller glia to cone photoreceptors in human retinal organoids with single MOGRIFY v.2.5 system transcription factors. Expression of single transcription factors was restricted to Müller glia using the truncated GFAP promoter (gfaABC1D), and successful conversions were determined by overlaying of GFAP_TFs_GFP and ARR3, a cone photoreceptor specific gene. (A and B) A control lentivirus vector with no transcription factor overexpression was used, where only GFP was driven by GFAP, and no co-localisation of GFP+ with ARR3+ cells was observed (white arrowheads illustrate where one would expect to see cell conversion and GFP/ARR3 overlays in the outer layer of the organoids, if MOGRIFY transcription factors were used). When retinal organoids are transduced with single transcription factors predicted by MOGRIFY v.2.5 system, including MEF2C (C and D); MEF2D (E and F); and RXRG (G and H), co-localisation of GFP and ARR3 was observed (white arrowheads illustrate green-GFP marked cone photoreceptor cells). Importantly, the morphology of converted cells was consistent with photoreceptors in the outer nuclear layer, where rudimentary outer segments were also observed.

FIG. 2. Design of a polycistronic lentivirus vector for MEF2C, MEF2D and RXRG.

FIG. 3. Reprogramming of Müller glia to cone photoreceptors in human retinal organoids with a polycistronic lentivirus vector encoding three MOGRIFY v.2.5 system transcription factors (MEF2C_MEF2D_RXRG). Expression of the three transcription factors was restricted to Müller glia using the truncated GFAP promoter, and successful conversions were determined by overlaying of GFAP_TF_GFP and ARR3, a cone photoreceptor specific gene. (A and B) No co-localisation of GFAP_GFP and ARR3 was observed when a control lentivirus vector with no transcription factor overexpression was used (white arrowheads illustrate where one would expect to see cell conversion in the outer layer of the organoids, if MOGRIFY transcription factors were used). On the other hand, when we used a polycistronic lentivirus vector in human retinal organoids (C and D), the conversion efficiency was qualitatively more efficient than when single transcription factors were used and GFP was expressed in a substantial proportion of cone photoreceptors (white arrowheads illustrate green-GFP marked cone photoreceptor cells).

FIG. 4. Quantification of total cone photoreceptors (ARR3+), total GFP+ cells, and reprogrammed cones (ARR3+/GFP+) in human retinal organoids after transduction with three MOGRIFY v.2.5 system transcription factors. No successful reprogramming of Müller glia to cones was observed when the control vector was used, however, when the polycistronic vector encoding three transcription factors was used, we observed successful reprogramming of Müller glia to cones (ARR3+/GFP+).

FIG. 5. Reprogramming of Müller glia to cone photoreceptors in a post-mortem mouse retinal explant. (A and B) When a control AAV was used that drives mCherry expression under the GFAP promoter, we observed no successful reprogramming events of Müller glia to cone photoreceptors (white arrowheads illustrate where one would expect to see cell conversion occurring). Alternatively, when an AAV was used that encodes two MOGRIFY v.2.5 system predicted transcription factors (MEF2D_CRX) (C and D), we observed co-localisation of mCherry with ARR3, illustrating that the transcription factors drive the conversion of Müller glia to cones.

FIG. 6. Quantification of total cone photoreceptors (ARR3+), total GFP+ cells, and reprogrammed cones (ARR3+/GFP+) in mouse retinal explants after transduction with AAVs encoding MOGRIFY v.2.5 system predicted factors. No successful reprogramming of Müller glia to cones was observed when the control vector was used, however, when bicistronic AAV vectors encoding two transcription factors (MEF2C_RXRG and MEF2D_CRX) were used, we observed successful reprogramming of Müller glia to cones (ARR3+/mCherry+).

FIGS. 7A and 7B. Human retinal explants were tested for light responsiveness using Multi Electrode Arrays (MEA) pre-transduction and 2 weeks post-transduction with either AAV_GFAP_mcherry (control, A) or with AAV_GFAP_MEF2C_RXRG and AAV_GFAP_MEF2D_CRX (TF treated, B). After 2-weeks post-transduction with MOGRIFY predicted transcription factors, there was an observed increase in light responses with blue light, when compared to the mCherry control which contains no transcription factors.

FIG. 8. The number of MEA channels registering mERGs was quantified. There are 60 channels on an MEA chip, however 1 is the reference electrode, therefore the maximum number of channels that can be recorded is 59. In A), where the explant was treated with the GFAP_mcherry control AAV, the number of channels recording mERG responses decreased over the 2 week culture period post transduction, whereas in B), where the explant was treated with the GFAP_ME2C_RXRG and GFAP_MEF2D_CRX AAVs, there is an increase in the number of channels recording mERGs.

FIG. 9. Signs of Müller glia to cone conversions are observed via observations of ARR3+ cells outside the outer nuclear layer following in vivo ocular delivery of the transcription factors. Immunohistochemical detection of ARR3 and mCherry in mouse retina sections from mice treated with bicistronic AAV vectors encoding two TFs (GFAP_MEF2C-RXRG_myc and GFAP_MEF2D_CRX_mCherry) shows that ARR3+ cells can be identified outside the photoreceptor layer (B and Bzoom). The cell body/nucleus of the ARR3+ cell shown here resides in the INL and not the ONL as expected. INL location is consistent with the location of Muller glia nuclei and thus suggesting a converting Muller glia cell. In further support of this, the cellular morphology of the ARR3+cells do not display a classical cone or Müller glia morphology. Overall, this is consistent with the Muller glia reprogramming events identified in the transduced retinal organoids and mouse retinal explants. Abbreviations: NFL: Nerve Fiber Layer, INL: Inner Nuclear Layer, ONL: Outer Nuclear Layer. A: DAPI, B and Bzoom: ARR3, C: mCherry, D: Overlay of DAPI, ARR3 and mCherry

FIG. 10. We observed a trend in which the number of ARR3+ cells outside the outer nuclear layer (ONL) increased the TF-treated retinas regardless of administration route (subretinal vs. Intravitreal).

FIG. 11. Volcano plot to highlight differentially expressed genes in individual TF conditions. Shown here are scatter plots of significance (pvalue on y axis) vs fold change in gene expression as a result of treatment with individual TFs. Control in each case is the same cell transduced with empty vector. Right upper and left upper quadrant dots (white with black outline) denote the upregulated and downregulated genes respectively; Black dots denote genes that did not show significant (p-adj <=0.05) differential (absolute log2FoldChange >1) expression. The number of significantly upregulated and downregulated genes are annotated in the right and left quadrants respectively.

FIG. 12. Heatmap to guide cell type identification of the transdifferentiated cells within the 2D system. Each row is a gene that is significantly (p-adj <=0.05) upregulated (absolute log2FoldChange >1) in one of the conditions (polycistronic, MEF2C only and CRX only) when compared to the same cell transduced with an empty vector. Illustrated is the normalised expression of the genes in an independent reference dataset (Cowan et al.). Along every row, darker shades denote higher expression of the gene in the corresponding cell types compared to the other cell types in the Cowan et al. dataset. Similarly, in each row, the lightest shade denotes the least expression of the gene in the corresponding cell type compared to other cell types in the Cowan dataset. MEF2D and RXRG conditions had no significantly upregulated genes.

FIG. 13. Heatmap of the log normalized expression of genes involved in retinal pigment epithelium (RPE) function in the untransduced control, polycistronic, MEF2C only, MEF2D only, RXRG only and CRX only cells in the 2D system. Within each control and treatment condition, the three columns denote the three experimental replicates.

FIG. 14. UMAP of retinal organoid scRNAseq data from control or TF-treated retinal organoid cells. Arrow highlights the RPE-like population found only in the TF-treated group.

FIG. 15. Boxplots depicting the expression of key RPE-marker genes, in the same retinal organoid scRNAseq data as shown in FIG. 14, across the annotated cell types (x axis) and conditions (black=control, grey=TF-treated).

FIG. 16. Heatmap of the average normalized expression of genes involved in retinal pigment epithelium (RPE) function in the control Müller glia and transcription factor (TF)-treated RPE-like populations. Expression data was measured by scRNAseq.

FIG. 17. Boxplot depicting the expression of Brain-Derived Neurotrophic Factor (BDNF), a key secreted factor of the retinal pigment epithelium, in cultured retinal organoid-derived Müller glia cells treated with polycistronic or monocistronic TF lentivirus vectors. Increased BDNF expression is seen from Müller glia cells treated with the polycistronic vector or the monocistronic MEF2C vector compared to other individual TFs and the no transduction and transduction controls. CRX does increase BDNF expression, however the effects of MEF2C alone and of the polycistronic vector are greater than that of CRX. The increase in BDNF expression with the polycistronic vector may represent a synergistic effect of MEF2C, MEF2D and RXRG since it appears to be greater than the additive effect of the three individual transcription factors. X-axis shows cellular treatment and y-axis the BDNF expression levels (transcripts per million).

FIG. 18. Quantification of secreted Brain-Derived Neurotrophic Factor (BDNF) levels from retinal organoid-derived Müller glia cells treated with either polycistronic lentivirus vector encoding three MOGRIFY v.2.5 system transcription factors (GFAP_MEF2C_MEF2D_RXRG_GFP) or GFP only (GFAP_GFP). BDNF secretion increased 4-fold in the Müller glia cells treated with the polycistronic lentivirus vector compared to the GFP only transduction control. Statistical test: unpaired t-test, p=0.0002

### DETAILED DESCRIPTION

According to a first aspect, the invention provides a nucleic acid molecule comprising a promoter operably linked to a nucleic acid sequence encoding Myocyte Enhancer Factor 2C (MEF2C), or a functional variant thereof, wherein the promoter is for expression of MEF2C in macroglia.

The data disclosed herein suggest that of the transcription factors tested, MEF2C surprisingly effects the higher number of gene expression changes, compared to CRX, MEF2D or RXRG, following administration to Müller glia cells. The data disclosed herein also suggest that MEF2C surprisingly has the greatest effect on RPE phagocytic genes and BDNF expression. MEF2C may therefore represent an advantageous single transcription factor to be used in transdifferentiation to RPE-like cells and/or cone-like photoreceptors and in treatments for associated retinal disease or degeneration.

The functional variant of MEF2C may comprise an amino acid sequence at least 70% identical to SEQ ID NO: 1. The functional variant of MEF2C may comprise an amino acid sequence at least 80% identical to SEQ ID NO: 1. The functional variant of MEF2C may comprise an amino acid sequence at least 85% identical to SEQ ID NO: 1. The functional variant of MEF2C may comprise an amino acid sequence at least 90% identical to SEQ ID NO: 1. The functional variant of MEF2C may comprise an amino acid sequence at least 95% identical to SEQ ID NO: 1. The functional variant of MEF2C may comprise an amino acid sequence at least 96% identical to SEQ ID NO: 1. The functional variant of MEF2C may comprise an amino acid sequence at least 97% identical to SEQ ID NO: 1. The functional variant of MEF2C may comprise an amino acid sequence at least 98% identical to SEQ ID NO: 1. The functional variant of MEF2C may comprise an amino acid sequence at least 99% identical to SEQ ID NO: 1. The nucleic acid sequence may comprise any nucleic acid sequence encoding a functional variant of MEF2C. Therefore, the nucleic acid sequence may comprise any nucleic acid sequence at least 70% identical to SEQ ID NO: 1. For example, the nucleic acid sequence may comprise any nucleic acid sequence at least 90% identical to SEQ ID NO: 1.

The nucleic acid sequence may comprise any nucleic acid sequence encoding MEF2C. The MEF2C may comprise an amino acid sequence according to SEQ ID NO: 1. Therefore, the nucleic acid sequence may comprise any nucleic acid sequence encoding SEQ ID NO: 1.

The nucleotide sequence encoding MEF2C or a functional variant thereof may comprise:
(a) SEQ ID NO: 3,
(b) SEQ ID NO: 2, or
(c) a nucleotide sequence having at least 60% identity to SEQ ID NO: 3 or SEQ ID NO: 2.

The nucleotide sequence having at least 60% identity to SEQ ID NO: 3 or SEQ ID NO: 2 may have at least 70%, 80%, 90% or 95% identity to SEQ ID NO: 3 or SEQ ID NO: 2. SEQ ID NO: 3 and SEQ ID NO: 2 have around 65% sequence identity to each other and encode the same protein sequence. The non-identical nucleotides may therefore represent silent mutations (i.e. mutations which do not alter the sequence of the encoded amino acid). Alternatively, the non-identical nucleotides may alter the amino acid sequence of the encoded amino acid, for example by one or more conservative amino acid mutation. The non-identical nucleotides may alter the amino acid sequence of the encoded amino acid by up to 20, up to 15, up to 10, up to 9, up to 8, up to 7, up to 6, up to 5, up to 4, up to 3, up to 2 or up to 1 conservative amino acid mutation.

The nucleic acid sequence may encode MEF2C. The nucleotide sequence encoding MEF2C may comprise:
(a) SEQ ID NO: 3, or
(b) SEQ ID NO: 2.

The nucleic acid molecule may comprise a promoter operably linked to a nucleic acid sequence encoding one or more transcription factor selected from the group consisting of Myocyte Enhancer Factor 2D (MEF2D), Retinoid X Receptor Gamma (RXRG) and Cone-Rod Homeobox (CRX), or functional variants thereof, or any combination thereof, wherein the promoter is for expression of the one or more transcription factor in macroglia. The promoter operably linked to MEF2D, RXRG and/or CRX may be the same or different to the promoter operably linked to MEF2C.

The nucleic acid molecule may encode a functional variant of MEF2D, RXRG and/or CRX.

The functional variant of MEF2D may comprise an amino acid sequence at least 70% identical to SEQ ID NO: 4. The functional variant of MEF2D may comprise an amino acid sequence at least 80% identical to SEQ ID NO: 4. The functional variant of MEF2D may comprise an amino acid sequence at least 85% identical to SEQ ID NO: 4. The functional variant of MEF2D may comprise an amino acid sequence at least 90% identical to SEQ ID NO: 4. The functional variant of MEF2D may comprise an amino acid sequence at least 95% identical to SEQ ID NO: 4. The functional variant of MEF2D may comprise an amino acid sequence at least 96% identical to SEQ ID NO: 4. The functional variant of MEF2D may comprise an amino acid sequence at least 97% identical to SEQ ID NO: 4. The functional variant of MEF2D may comprise an amino acid sequence at least 98% identical to SEQ ID NO: 4. The functional variant of MEF2D may comprise an amino acid sequence at least 99% identical to SEQ ID NO: 4. The nucleic acid sequence may comprise any nucleic acid sequence encoding a functional variant of MEF2D. Therefore, the nucleic acid sequence may comprise any nucleic acid sequence at least 70% identical to SEQ ID NO: 4. For example, the nucleic acid sequence may comprise any nucleic acid sequence at least 90% identical to SEQ ID NO: 4.

The nucleic acid sequence may comprise any nucleic acid sequence encoding MEF2D. The MEF2D may comprise an amino acid sequence according to SEQ ID NO: 4. Therefore, the nucleic acid sequence may comprise any nucleic acid sequence encoding SEQ ID NO: 4.

The nucleotide sequence encoding MEF2D or a functional variant thereof may comprise:
(a) SEQ ID NO: 6,
(b) SEQ ID NO: 5, or
(c) a nucleotide sequence having at least 60% identity to SEQ ID NO: 6 or SEQ ID NO: 5.

The nucleotide sequence having at least 60% identity to SEQ ID NO: 6 or SEQ ID NO: 5 may have at least 70%, 80%, 90% or 95% identity to SEQ ID NO: 6 or SEQ ID NO: 5. The non-identical nucleotides may represent silent mutations (i.e. mutations which do not alter the sequence of the encoded amino acid). Alternatively, the non-identical nucleotides may alter the amino acid sequence of the encoded amino acid, for example by one or more conservative amino acid mutation. The non-identical nucleotides may alter the amino acid sequence of the encoded amino acid by up to 20, up to 15, up to 10, up to 9, up to 8, up to 7, up to 6, up to 5, up to 4, up to 3, up to 2 or up to 1 conservative amino acid mutation.

The nucleic acid sequence may encode MEF2D. The nucleotide sequence encoding MEF2D may comprise:
(a) SEQ ID NO: 6, or
(b) SEQ ID NO: 5.

The functional variant of RXRG may comprise an amino acid sequence at least 70% identical to SEQ ID NO: 7. The functional variant of RXRG may comprise an amino acid sequence at least 80% identical to

SEQ ID NO: 7. The functional variant of RXRG may comprise an amino acid sequence at least 85% identical to SEQ ID NO: 7. The functional variant of RXRG may comprise an amino acid sequence at least 90% identical to SEQ ID NO: 7. The functional variant of RXRG may comprise an amino acid sequence at least 95% identical to SEQ ID NO: 7. The functional variant of RXRG may comprise an amino acid sequence at least 96% identical to SEQ ID NO: 7. The functional variant of RXRG may comprise an amino acid sequence at least 97% identical to SEQ ID NO: 7. The functional variant of RXRG may comprise an amino acid sequence at least 98% identical to SEQ ID NO: 7. The functional variant of RXRG may comprise an amino acid sequence at least 99% identical to SEQ ID NO: 7. The nucleic acid sequence may comprise any nucleic acid sequence encoding a functional variant of RXRG. Therefore, the nucleic acid sequence may comprise any nucleic acid sequence at least 70% identical to SEQ ID NO: 7. For example, the nucleic acid sequence may comprise any nucleic acid sequence at least 90% identical to SEQ ID NO: 7.

The nucleic acid sequence may comprise any nucleic acid sequence encoding RXRG. The RXRG may comprise an amino acid sequence according to SEQ ID NO: 7. Therefore, the nucleic acid sequence may comprise any nucleic acid sequence encoding SEQ ID NO: 7.

The nucleotide sequence encoding RXRG or a functional variant thereof may comprise:
(a) SEQ ID NO: 9,
(b) SEQ ID NO: 8, or
(c) a nucleotide sequence having at least 60% identity to SEQ ID NO: 9 or SEQ ID NO: 8.

The nucleotide sequence having at least 60% identity to SEQ ID NO: 9 or SEQ ID NO: 8 may have at least 70%, 80%, 90% or 95% identity to SEQ ID NO: 9 or SEQ ID NO: 8. The non-identical nucleotides may represent silent mutations (i.e. mutations which do not alter the sequence of the encoded amino acid). Alternatively, the non-identical nucleotides may alter the amino acid sequence of the encoded amino acid, for example by one or more conservative amino acid mutation. The non-identical nucleotides may alter the amino acid sequence of the encoded amino acid by up to 20, up to 15, up to 10, up to 9, up to 8, up to 7, up to 6, up to 5, up to 4, up to 3, up to 2 or up to 1 conservative amino acid mutation.

The nucleic acid sequence may encode RXRG. The nucleotide sequence encoding RXRG may comprise:
(a) SEQ ID NO: 9, or
(b) SEQ ID NO: 8.

The functional variant of CRX may comprise an amino acid sequence at least 70% identical to SEQ ID NO: 10. The functional variant of CRX may comprise an amino acid sequence at least 80% identical to SEQ ID NO: 10. The functional variant of CRX may comprise an amino acid sequence at least 85% identical to SEQ ID NO: 10. The functional variant of CRX may comprise an amino acid sequence at least 90% identical to SEQ ID NO: 10. The functional variant of CRX may comprise an amino acid sequence at least 95% identical to SEQ ID NO: 10. The functional variant of CRX may comprise an amino acid sequence at least 96% identical to SEQ ID NO: 10. The functional variant of CRX may comprise an amino acid sequence at least 97% identical to SEQ ID NO: 10. The functional variant of CRX may comprise an amino acid sequence at least 98% identical to SEQ ID NO: 10. The functional variant of CRX may comprise an amino acid sequence at least 99% identical to SEQ ID NO: 10. The nucleic acid sequence may comprise any nucleic acid sequence encoding a functional variant of CRX. Therefore, the nucleic acid sequence may comprise any nucleic acid sequence at least 70% identical to SEQ ID NO: 10. For example, the nucleic acid sequence may comprise any nucleic acid sequence at least 90% identical to SEQ ID NO: 10.

The nucleic acid sequence may comprise any nucleic acid sequence encoding CRX. The CRX may comprise an amino acid sequence according to SEQ ID NO: 10. Therefore, the nucleic acid sequence may comprise any nucleic acid sequence encoding SEQ ID NO: 10.

The nucleotide sequence encoding CRX or a functional variant thereof may comprise:
(a) SEQ ID NO: 12,
(b) SEQ ID NO: 11, or
(c) a nucleotide sequence having at least 60% identity to SEQ ID NO: 12 or SEQ ID NO: 11.

The nucleotide sequence having at least 60% identity to SEQ ID NO: 12 or SEQ ID NO: 11 may have at least 70%, 80%, 90% or 95% identity to SEQ ID NO: 12 or SEQ ID NO: 11. The non-identical nucleotides may represent silent mutations (i.e. mutations which do not alter the sequence of the encoded amino acid). Alternatively, the non-identical nucleotides may alter the amino acid sequence of the encoded amino acid, for example by one or more conservative amino acid mutation. The non-identical nucleotides may alter the amino acid sequence of the encoded amino acid by up to 20, up to 15, up to 10, up to 9, up to 8, up to 7,up to 6, up to 5, up to 4, up to 3, up to 2 or up to 1 conservative amino acid mutation.

The nucleic acid sequence may encode CRX. The nucleotide sequence encoding CRX may comprise:
(a) SEQ ID NO: 12, or
(b) SEQ ID NO: 11.

The nucleic acid molecule may comprise a nucleotide sequence encoding MEF2C and a nucleotide sequence encoding RXRG. The combination of MEF2C and RXRG may mediate conversion of Müller glia to cone-like photoreceptors independently of MEF2D and CRX. The nucleic acid molecule may be a bicistronic molecule comprising a nucleotide sequence encoding MEF2C and a nucleotide sequence encoding RXRG. The nucleic acid molecule may encode a functional variant of MEF2C and/or a functional variant of RXRG. The nucleic acid molecule may encode RXRG and a functional variant of MEF2C. The nucleic acid molecule may encode MEF2C and a functional variant of RXRG. The nucleic acid molecule may encode a functional variant of MEF2C and a functional variant of RXRG.

The nucleic acid molecule may comprise a nucleotide sequence encoding MEF2C, a nucleotide sequence encoding MEF2D and a nucleotide sequence encoding RXRG. The data disclosed herein suggest that the combination of MEF2C, MEF2D and RXRG effect synergistic gene expression changes, compared to the individual transcription factors, following administration to Müller glia cells. The data disclosed herein also suggest that MEF2C, MEF2D and RXRG surprisingly upregulate a variety of genes associated with RPE functions, as well as BDNF expression and secretion. MEF2C, MEF2D and RXRG may therefore represent an advantageous combination of transcription factors to be used in transdifferentiation to RPE-like cells and/or cone-like photoreceptors and in treatments for associated retinal disease or degeneration. Without being bound by theory, one or more effects of the combination of MEF2C, MEF2D and RXRG may be similarly observed with any combination of two of the three transcription factors. The nucleic acid molecule may therefore comprise a nucleotide sequence encoding MEF2C and a nucleotide sequence encoding MEF2D. The nucleic acid molecule may therefore comprise a nucleotide sequence encoding MEF2C and a nucleotide sequence encoding RXRG. In an alternative statement, the nucleic acid molecule may therefore comprise a nucleotide sequence encoding MEF2D and a nucleotide sequence encoding RXRG. The nucleic acid molecule may be a polycistronic molecule comprising a nucleotide sequence encoding MEF2C, a nucleotide sequence encoding MEF2D and a nucleotide sequence encoding RXRG. The nucleic acid molecule may encode a functional variant of one or more of MEF2C, MEF2D and/or RXRG. The nucleic acid molecule may encode MEF2D, RXRG and a functional variant of MEF2C. The nucleic acid molecule may encode MEF2C, MEF2D and a functional variant of RXRG. The nucleic acid molecule may encode MEF2C, RXRG and a functional variant of MEF2D. The nucleic acid molecule may encode MEF2D, a functional variant of RXRG and a functional variant of MEF2C. The nucleic acid molecule may encode MEF2C, a functional variant of MEF2D and a functional variant of RXRG. The nucleic acid molecule may encode RXRG, a functional variant of MEF2C and a functional variant of MEF2D. The nucleic acid molecule may encode a functional variant of RXRG, a functional variant of MEF2C and a functional variant of MEF2D.

The MEF2C, MEF2D and RXRG may be in any suitable arrangement within the nucleic acid molecule. The nucleic acid sequence encoding MEF2C may be 5′ relative to a nucleic acid sequence encoding one or more further transcription factor, such as MEF2D and/or RXRG. A nucleic acid sequence encoding RXRG may be 3′ relative to a nucleic acid sequence encoding MEF2C. The nucleic acid sequence encoding MEF2C may be 5′ relative to a nucleic acid sequence encoding MEF2D and a nucleic acid sequence encoding RXRG may be 3′ relative to the nucleic acid sequence encoding MEF2D. The nucleic acid molecule may have the arrangement 5′—MEF2C—MEF2D-RXRG—3′.

The nucleic acid molecule may comprise a nucleotide sequence encoding MEF2C, a nucleotide sequence encoding MEF2D and a nucleotide sequence encoding RXRG, and optionally a nucleotide sequence encoding CRX. The nucleic acid molecule may encode a functional variant of one or more of MEF2C, MEF2D and/or RXRG, and optionally CRX. The nucleic acid molecule may encode MEF2D, RXRG and a functional variant of MEF2C, and optionally CRX. The nucleic acid molecule may encode MEF2C, MEF2D and a functional variant of RXRG, and optionally CRX. The nucleic acid molecule may encode MEF2C, RXRG and a functional variant of MEF2D, and optionally CRX. The nucleic acid molecule may encode MEF2C, RXRG and MEF2D, and optionally a functional variant of CRX.

The promoter may be selected from the group consisting of Glial fibrillary acidic protein (GFAP), CAR2, CD44, GLUL, PDGFRA, retinaldehyde-binding protein 1 (RLBP1), S100B, SLC1A3, VIM, ProB2, GLAST, CAG and CMV. The promoter may be selected from the group consisting of GFAP, RLBP1, ProB2 and GLAST. The promoter may be a macroglia specific promoter. The macroglia may be retinal macroglia. The macroglia may be Müller glia and/or astrocytes. The macroglia may be Müller glia. The promoter may be a GFAP promoter. The promoter may be a gfaABC1D GFAP promoter. The GFAP promoter may consist of or may comprise SEQ ID NO: 13. The GFAP promoter may consist of or may comprise SEQ ID NO: 14.

According to a second aspect, the invention provides a vector comprising the nucleic acid molecule according to the first aspect.

The vector may be a viral vector. The viral vector may be selected from the group consisting of a lentiviral vector, a Sendai vector, a Herpes simplex virus (HSV) vector, an Adenoviral vector, an adeno-associated virus (AAV) vector, an episomal vector and a retroviral vector. The viral vector may be selected from the group consisting of a lentiviral vector, an Adenoviral vector and an adeno-associated virus (AAV) vector. The viral vector may be an AAV vector. The viral vector may be a lentiviral vector.

The vector may be a non-viral vector. The non-viral vector may be selected from the group consisting of a liposome, nanoparticle, naked DNA, plasmid and a transposon. The non-viral vector may be a repRNA vector or mRNA.

According to a third aspect, the invention provides a composition comprising the nucleic acid molecule according to the first aspect or the vector according to the second aspect, and a pharmaceutically acceptable carrier.

Pharmaceutically acceptable carriers suitable for the delivery of compositions of the present invention and methods for their preparation will be readily apparent to those skilled in the art.

According to a fourth aspect, the invention provides a product comprising
(a) a first nucleic acid molecule, according to the first aspect, and
(b) a second nucleic acid molecule, comprising a promoter operably linked to a nucleic acid sequence encoding one or more transcription factor selected from the group consisting of Myocyte Enhancer Factor 2D (MEF2D), Retinoid X Receptor Gamma (RXRG) and Cone-Rod Homeobox (CRX), or functional variants thereof, or any combination thereof, wherein the promoter is for expression of the one or more transcription factor in macroglia;
   as a combined preparation for simultaneous, separate or sequential use in the treatment of retinal disease or degeneration.

The product may be one or more nucleic acid molecules that between them encode MEF2C or a functional variant thereof and one or more transcription factor. The one or more transcription factor may be selected from the group consisting of MEF2D, RXRG and CRX, or functional variants thereof. The one or more transcription factor may be selected from the group consisting of MEF2D and RXRG, or functional variants thereof.

The product may be one or more nucleic acid molecules that between them encode MEF2C and one or more transcription factor. The one or more transcription factor may be selected from the group consisting of MEF2D, RXRG and CRX. The one or more transcription factor may be selected from the group consisting of MEF2D and RXRG.

At least one of the first nucleic acid molecule and the second nucleic acid molecule may encode MEF2D or a functional variant thereof and at least one of the first nucleic acid molecule and the second nucleic acid molecule may encode RXRG or a functional variant thereof. At least one of the first nucleic acid molecule and the second nucleic acid molecule may encode MEF2D and at least one of the first nucleic acid molecule and the second nucleic acid molecule may encode RXRG.

At least one or more of the first nucleic acid molecule and the second nucleic acid molecule may further encode CRX or a functional variant thereof. At least one of the first nucleic acid molecule and the second nucleic acid molecule may further encode CRX or a functional variant thereof.

In some instances,
(a) the first nucleic acid molecule comprises a nucleic acid sequence encoding MEF2C and a nucleic acid sequence encoding RXRG, and/or
(b) the second nucleic acid molecule comprises a nucleic acid sequence encoding MEF2D and a nucleic acid sequence encoding CRX.

In some instances,
(a) the first nucleic acid molecule comprises a nucleic acid sequence encoding MEF2C and a nucleic acid sequence encoding RXRG, and
(b) the second nucleic acid molecule comprises a nucleic acid sequence encoding MEF2D and a nucleic acid sequence encoding CRX.

The first and/or second nucleic acid molecule may be in the form of a vector or a composition. For example, the first nucleic acid molecule may be in the form of a vector of the second aspect of the invention or in the form of a composition of the third aspect of the invention. Likewise, the second nucleic acid molecule may be in the form of a vector of the second aspect of the invention or in the form of a composition of the third aspect of the invention, wherein the second nucleic acid molecule optionally may not encode MEF2C and may instead encode one or more other transcription factor, which may be selected from the group consisting of MEF2D, RXRG and CRX. Any one or more of the MEF2C, MEF2D, RXRG and/or CRX may be replaced by a functional variant thereof.

The product may comprise a third nucleic acid molecule. For instance, the first, second and third nucleic acid molecules may between them encode MEF2C and two or more transcription factors. The two or more transcription factors may be selected from the group consisting of MEF2D, RXRG and CRX. The two or more transcription factors may be selected from the group consisting of MEF2D and RXRG. For instance, the first nucleic acid molecule may encode MEF2C, the second nucleic acid molecule may encode MEF2D and the third nucleic acid molecule may encode RXRG. Any one or more of the MEF2C, MEF2D, RXRG and/or CRX may be replaced by a functional variant thereof.

The third nucleic acid molecule may be in the form of a vector or a composition. For example, the third nucleic acid molecule may be in the form of a vector of the second aspect of the invention or in the form of a composition of the third aspect of the invention. Likewise, the third nucleic acid molecule may be in the form of a vector of the second aspect of the invention or in the form of a composition of the third aspect of the invention, wherein the third nucleic acid molecule optionally may not encode MEF2C and may instead encode one or more other transcription factor, which may be selected from the group consisting of MEF2D, RXRG and CRX. Any one or more of the MEF2C, MEF2D, RXRG and/or CRX may be replaced by a functional variant thereof.

The product may comprise a fourth nucleic acid molecule. For instance, the first, second, third and fourth nucleic acid molecules may between them encode MEF2C and three or more transcription factors. The three or more transcription factors may be selected from the group consisting of MEF2D, RXRG and CRX. The three or more transcription factors may comprise MEF2D, RXRG and CRX. For instance, the first nucleic acid molecule may encode MEF2C, the second nucleic acid molecule may encode MEF2D, the third nucleic acid molecule may encode RXRG and the fourth nucleic acid molecule may encode CRX. Any one or more of the MEF2C, MEF2D, RXRG and/or CRX may be replaced by a functional variant thereof.

The fourth nucleic acid molecule may be in the form of a vector or a composition. For example, the fourth nucleic acid molecule may be in the form of a vector of the second aspect of the invention or in the form of a composition of the third aspect of the invention. Likewise, the fourth nucleic acid molecule may be in the form of a vector of the second aspect of the invention or in the form of a composition of the third aspect of the invention, wherein the fourth nucleic acid molecule optionally may not encode MEF2C and may instead encode one or more other transcription factor, which may be selected from the group consisting of MEF2D, RXRG and CRX.

The first, second, third or fourth nucleic acid molecule may together encode one or more of MEF2C, MEF2D, RXRG and/or CRX in any combination. The first, second, third or fourth nucleic acid molecule may encode a functional variant of a transcription factor disclosed herein, typically in place of the corresponding transcription factor. The first, second, third or fourth nucleic acid molecule may encode a functional variant of one or more of MEF2C, MEF2D, RXRG and/or CRX in any combination. The first, second, third or fourth nucleic acid molecule may together encode one or more of MEF2C, MEF2D, RXRG and/or CRX in any combination and/or a functional variant of one or more of MEF2C, MEF2D, RXRG and/or CRX in any combination.

The product may optionally further comprise instructions for the simultaneous, sequential or separate administration of the preparations to a subject in need thereof.

It is to be appreciated that all references herein to treatment include curative, palliative and prophylactic treatment; although in the context of the invention references to preventing are more commonly associated with prophylactic treatment. Treatment may also include arresting progression in the severity of a disease.

The treatment of mammals, particularly humans, is preferred. However, both human and veterinary treatments are within the scope of the invention.

The treatment may comprise may comprise administration by intravitreal, suprachoroidal or subretinal injection.

The term “combination”, or terms “in combination”, “used in combination with” or “combined preparation” as used herein may refer to the combined administration of two or more agents simultaneously, sequentially or separately.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5minutes or 10 minutes after the other.

According to a fifth aspect, the invention provides a method of comverting a retinal source cell to a retinal target cell by inttroducing one or more transciprtion factor comprising MEF2C, or a functional variant thereof, into the retinal souorce cell, thereby converting the retinal source cell into the retinal target cell.

The retinal souorce cell may be a macroglia. The retinal source cell may be a Müglia cell or astrocyte. The retinal source cell may be a population of macroglia. The retinal source cell may be a population of Mü glia cell or a populational of astrocytes.

The retinal target cell may be a photoreceptor-like cell or a retinal pigment epithelilum (RPE) like cell. The retinal taarget cell may be a opulation of a photoreceptor-like cells or a population of retinal pigment epithelium (RPE)-like cells. The photoreceptorlike cell may be a cone-like photoceptor cell. The population of photoreceptor-like cells may be a population of cone-like photoreceptor cells.

Alternatively, the method may be described as a method of converting a source cell to a target cell by introducing one or more transciption facttor comprising MEF2C, or a functional variant thereof, into the source cell, thereby converting the source cell into the target cell. The method and/or the target cell provided by the method may be for treating a retinal disease or degeneration.

In some embodiments of the method, the one or more transcription factor is introduced via a nucleic acid molecule according to the first aspect, a vector according to the second aspect, a composition according to the third aspect or a product according to the fourth aspect.

The method may be an in vitro method. The method may comprise culturing under suitable conditions for at least 2 days. The method may comprise culturing under suitable conditions for at least 4 days. The method may comprise culturing under suitable conditions for at least 7 days. The culturing may be culturing after transduction.

According to a sixth aspect, the invention provides a cell produced by the method of the fifth aspect. The cell may have any one or more of the characteristics of the cells produced by the methods described herein. The cell may have any one or more of the characteristics of a cone-like photoreceptor described herein. The cell may have any one or more of the characteristics of an RPE-like cell described herein. The cell may be a non-natural cell. The cell may lack any one or more of the characteristics of the target cell. The cell may lack any one or more of the characteristics of a cone photoreceptor described herein. The cell may lack any one or more of the characteristics of an RPE cell described herein.

According to a seventh aspect, the invention provides the nucleic acid molecule according to the first aspect, the vector according to the second aspect, the composition according to the third aspect, or the cell according to the sixth aspect for use in the treatment of retinal disease or degeneration.

According to an eighth aspect, the invention provides a method of treating retinal disease or degeneration in a subject comprising administering to a retina of the subject in need thereof a therapeutically effective amount of the nucleic acid molecule according to the first aspect, the vector according to the second aspect, the composition according to the third aspect, the product according to the fourth aspect, or the cell according to the sixth aspect.

The retinal disease or degeneration may be any retinal disease or degeneration characterised by a loss of photoreceptors, especially cone photoreceptors and/or RPE cells. The retinal disease or degeneration may be selected from the group consisting of age-related macular degeneration (AMD), retinitis pigmentosa (RP), late-stage Best disease, Stargadt macular dystrophy, cone rod dystrophy or glaucoma. The retinal disease or degeneration may be age-related macular degeneration (AMD). The retinal disease or degeneration may be retinitis pigmentosa (RP). The retinal disease or degeneration may be late-stage Best disease. The retinal disease or degeneration may be Stargadt macular dystrophy. The retinal disease or degeneration may be cone rod dystrophy. The retinal disease or degeneration may be glaucoma.

The retinal disease or degeneration may be age-related macular degeneration (AMD). The AMD may be dry AMD. The dry AMD is late-stage dry AMD. The late-stage dry AMD may be geographic atropy or the late-stage dry AMD may be characterised by geographic atropy. The retinal disease or degeneration may be geographic atrophy. Geographic atrophy may be a symptom of the retinal disease or degeneration. Geographic atrophy may be a symptom of the late-stage dry AMD.

The present disclosure provides methods for converting source cells, such as Müller glia cells, to cone photoreceptor cells. Methods for converting include direct cell conversion or transdifferentiation of a source cell to a cone photoreceptor. The inventors have identified that increasing the expression and/or amount of at least one or more transcription factors, or variants thereof, selected from MEF2C, MEF2D and RXRG, and optionally CRX, can transdifferentiate a source cell, selected from a Müller glia or astrocyte cell, to a cone photoreceptor. The method may comprise increasing the expression and/or amount of at least one or more transcription factors, or variants thereof, comprising MEF2C, preferably further comprising MEF2D and/or RXRG, and optionally further comprising CRX, to transdifferentiate a source cell, selected from a Müller glia or astrocyte cell, to a cone-like photoreceptor cell.

The present disclosure further provides methods for converting source cells, such as Müller glia cells, to Retinal Pigment Epithelium (RPE)-like cells. Methods for converting include direct cell conversion or transdifferentiation of a source cell to an RPE-like cell. The inventors have identified that increasing the expression and/or amount of at least one or more transcription factors, or variants thereof, comprising MEF2C, preferably further comprising MEF2D and/or RXRG, and optionally further comprising CRX, can transdifferentiate a source cell, selected from a Muller glia or astrocyte cell, to an RPE-like cell.

As used herein, a “source cell” is the starting cell in a cell conversion process. A “source cell” as used by the present invention may be primary cells (non-immortalized cells), or may be derived from a cell line (immortalized cells). A source cell may refer to a population of cells, for example a mixed population of cells such as those obtained from a tissue e.g. retinal organoids, or cadaveric retinal explants from mice or humans. “A source cell” as used herein is a somatic cell which is a cell derived from one of the germ layers (ectoderm, endoderm or mesoderm). In some embodiments, a source cell is a progenitor cell, a differentiated cell, or a transdifferentiating cell. A differentiated cell, also known as a terminally differentiated cell, typically does not have the ability to divide to produce offspring cell. Differentiated cells are specialised cells that are designed to carry out a particular role in the body. A transdifferentiating cell is a cell that is undergoing the process of transdifferentiation i.e. the phenotype of the differentiated cell is changing towards the phenotype of another differentiated cell wherein the markers of the final differentiated cell have not yet been fully established. Preferably, the source cells according to the present invention are macroglia cells selected from the group comprising a Müller glia (MG) cell and an astrocyte cell.

Müller glia cells are a type of supportive glial cell specifically found in the retina, but they are not directly involved in the light transduction pathway. In Zebrafish (a vertebrate), Müller glia are capable of renewing photoreceptors after retinal injury, suggesting that they are a form of adult retinal progenitor cell in lower order species. This regenerative capacity is also observed in the early post-development of chicken retinas (avian species). However, this ability is lost in mammals as the retina is unable to regenerate post-development, so any injury or disease has severe consequences on vision. In 2017, Yao et al. (Nature) provided the first evidence of in vivo transdifferentiating Muller glia to rod photoreceptors to restore the vision of blind mice. However, rods are involved in peripheral and night vision, whereas humans are more dependent on cone photoreceptors for central visual acuity, so there is more of a need to identify means to transdifferentiate Müller glia cells to cone photoreceptors for regenerative approaches. No one to this point has used Muller glia cells for the re-creation of cone photoreceptor cells. Such methods are a tremendous advantage over the use of other cells, such as induced or embryonic stem cells, because they can be converted in vivo, eliminating any possibility of rejection problems and, being already in situ, they have the interconnecting pathways between other cells of the retinal tissue that are vital for the functional operation of the cells.

As used herein, “Müller glia cells” is a reference to any cell that has the characteristics of a Müller glia cell. This may be based on the presence of at least one marker and/or at least one morphological trait of a Müller glia cell. At least one marker of a Müller glia cell is selected from Glial fibrillary acidic protein (GFAP), Mu-crystallin homolog (CRYM), or glutamine synthase (GLUL) or combinations thereof. In an intact retina, the nuclei of Muller glia cells are anatomically positioned in the INL (Inner Nuclear Layer), while the cell spans across the entire neural retina. The Muller glia cell may be a healthy cell or activated through disease, such as inherited retinal degeneration, or age-related macular degeneration. In retinal degeneration, photoreceptor loss leads to reactive gliosis, an upregulation of GFAP, and proliferation of Müller glia cells. A Müller glia cell or population of Muller glia cells of the present invention are typically mammalian cells, such as, human cells, primate cells, rodent cells (e.g. mouse or rat) and bovine cells, preferably human cells. “A Müller glia cell” as used by the present invention encompasses a Müller glia cell or a population of Müller glia cells.

Astrocytes are a type of glial cell that are not derived from retinal progenitor cells, unlike Müller glia, but are also derived from a neuro-ectoderm lineage. Müller glia and astrocytes form a population of cells known as macroglia, which differ in origin and function from the microglia. Retinal astrocytes originate from the brain and optic nerve, invading the retina in development and, therefore, they are not present in human retinal organoids. Astrocytes constitutively express the intermediate filament glial fibrillary acidic protein (GFAP), which is only expressed in Müller glia cells upon activation through injury or disease. Therefore, it is believed that methods according to the present invention will also convert astrocytes in the human retina as they express GFAP and will also overexpress the exogenous transcription factors.

Photoreceptors are located in the neural retina, which lines the back of the eye. Photoreceptors respond to light and play a vital role in vision. There are two types of photoreceptors: cone and rod cells. Rod photoreceptors are highly sensitive and function in dim light to mediate night vision; they use rhodopsin as the light-sensitive photopigment. Cone photoreceptors are present as several subtypes, each of which contains a specific opsin that is maximally sensitive to a different wavelength of light; cones thus mediate colour perception. There are three subtypes of cone cells according to opsin expression: (short-, medium-, long-wavelength; blue, green, and red). Cone cells are fewer in numbers compared to rod cells and are cone shaped. Both cones and rods are located in the ONL (outer nuclear layer) of the retina. Of the two types of photoreceptors, cone cells are crucial for central, high acuity vision in humans.

“Cone photoreceptor cell” or “cone-like photoreceptor cell” as used in the present disclosure are any cells that have the characteristics of a cone photoreceptor cell. This may be based on the presence of at least one marker and/or at least one morphological trait of a cone photoreceptor cell. Cone morphology is well described in the literature (Daniele et al., IOVS 2005); they exhibit tightly packed discs enriched with photopigments (opsins) that constitute their outer segments. The inner segments contain numerous mitochondria, and they have synaptic terminals that interact with interneurons to transmit the phototransduction signals. At least one specific marker of a cone photoreceptor cell is selected from: Cone arrestin (ARR3), GNAT2, and one of S/M/L opsins, and combinations thereof. At least one morphological trait of a cone photoreceptor cell is selected from: a cone shaped outer segment filled with light sensitive S-, M- or L-opsins for phototransduction, a connecting cilium between the inner and outer segments, an inner segment filled with ribosomes and mitochondria for biosynthesis and transport of opsins, a cell body containing a nucleus, and an axon with a synaptic terminal to allow for neurotransmission. Preferably, the cone photoreceptor cell features two or more morphological traits. The morphological trait of particular relevance for the skilled person to identify the generation of an effective cone photoreceptor cell that is able to detect light is the development of a defined cone-shaped outer segment. “A cone photoreceptor” as used by the present invention encompasses a cone photoreceptor or a population of cone photoreceptors. Since it is not technically viable to show in every instance that a “cone photoreceptor cell” produced as described herein exhibits all of the characteristics of a “natural” cone photoreceptor cell, the terms “cone photoreceptor cell” and “cone-like photoreceptor cell” are used interchangeably herein.

“RPE cell” or “RPE-like cell” as used in the present disclosure are any cells that have the characteristics of an RPE cell. This may be based on the presence of at least one marker and/or at least one morphological trait and/or at least one functional characteristic of an RPE cell. RPE morphology is well described in the literature. RPE morphology may comprise the presence of pigment-containing melanosomes and/or an epithelial phenotype and/or monolayer formation and/or polarisation. The at least one marker for an RPE cell may be selected from the group consisting of BEST1, MITF, ITGAV, ITGB5, MFGE8, CD81, MERTK, GAS6, PTK2, RLBP1, RPE65, LRAT, RDH5, RDH10, RDH11, PMEL, TYR, TYRP1, GPR143, DCT, OCA2, RAB38 and MYRIP. The at least one marker may be any 1, 2, 3, 4, 5, 6, 7, 8, 9, 10, 11, 12, 13, 14, 15, 16, 17, 18, 19, 20, 21, 22 or 23 of BEST1, MITF, ITGAV, ITGB5, MFGE8, CD81, MERTK, GAS6, PTK2, RLBP1,

RPE65, LRAT, RDH5, RDH10, RDH11, PMEL, TYR, TYRP1, GPR143, DCT, OCA2, RAB38 and MYRIP, including any combinations thereof. The at least one marker for an RPE cell may be selected from the group consisting of RPE-65, BEST1, MITF and PMEL. The at least one marker for an RPE cell may be any 1, 2, 3 or 4 of RPE-65, BEST1, MITF and PMEL, including any combinations thereof. The RPE-like cell may express RPE-65, BEST1, MITF and/or PMEL accordingly. For example, the RPE-like cell may express

BEST1, MITF and PMEL. The at least one morphological trait of an RPE cell may be selected from: pigment-containing melanosomes, an epithelial phenotype, monolayer formation and/or polarisation. The at least one functional characteristic of an RPE cell may be selected from: phagocytosis of cone photoreceptor outer segments; visual cycle (a biochemical processes that regenerates chromophore); and pigmentation. Preferably, the RPE-like cell features two or more functional characteristics of an RPE cell.

An RPE-like cell may express genes associated with one or more functional characteristic of an RPE cell. An RPE-like cell may express genes associated with phagocytosis of cone photoreceptor outer segments. The genes associated with phagocytosis of cone photoreceptor outer segments may be selected from the group consisting of ITGAV, ITGB5, MFGE8, CD81, MERTK, GAS6 and PTK2. The genes associated with phagocytosis of cone photoreceptor outer segments may be associated with the binding phase of phagocytosis. The genes associated with the binding phase of phagocytosis may be selected from the group consisting of ITGAV, ITGB5, MFGE8 and CD81. The genes associated with the phagocytosis of cone photoreceptor outer segments may be associated with the internalisation phase of phagocytosis. The genes associated with the internalisation phase of phagocytosis may be selected from the group consisting of MERTK, GAS6 and PTK2. An RPE-like cell may express genes associated with the visual cycle. The genes associated with the visual cycle may be selected from the group consisting of RLBP1, RPE65, LRAT, RDH5, RDH10 and RDH11. An RPE-like cell may express genes associated with pigmentation. The genes associated with pigmentation may be selected from the group consisting of include PMEL, TYR, TYRP1, GPR143, DCT, OCA2, RAB38 and MYRIP. “An RPE cell” as used by the present invention encompasses an RPE cell or a population of RPE cells. Since it is not technically viable to show in every instance that a “RPE cell” produced as described herein exhibits all of the characteristics of a “natural” RPE cell, the terms “RPE cell” and “RPE-like cell” are used interchangeably herein.

As used herein, “cell conversion” refers to a method of “transdifferentiation” or “direct cell conversion”, sometimes termed “forward programming” i.e. changing the morphology and/or phenotype from a terminally differentiated cell such as the source cell of the present invention to another terminally differentiated cell. The process of transdifferentiation can be either “direct”, without going through an intermediate pluripotent state or progenitor cell type, or “indirect”, where the process goes through an intermediate pluripotent state or progenitor cell type. Methods of inducing “indirect” transdifferentiation can be recognized by the process involving transcription factors associated with a pluripotency phase, for example, the Yamanaka factors OCT3, OCT4 (also known as POU5F1), SOX2, KLF4 and cMYC are known to be highly expressed in embryonic stem(ES) cells. Further pluripotent transcription factors that are well known to contribute to the reprogramming of somatic cells to a pluripotent-like state include NANOG, LIN28 and GLIS1. The term “cell conversion” may be used interchangeably with the terms “transdifferentiation” or “direct cell conversion”. Advantageously, “cell conversion” enables the conversion of a cell derived from a lineage to a cell derived from the same or different lineage. For example, the present invention converts a source cell, selected from an astrocyte or Müller glia cell, which are derived from the neuro-ectoderm lineage, to cone photoreceptors, which are also neuro-ectoderm derived.

Without being bound by theory, the transcription factors disclosed herein may convert a macroglial source cell to a cone-like photoreceptor cell and/or to an RPE-like cell via a progenitor cell. Since the transcription factors are not pluripotency inducing, any such progenitor cell will not be a pluripotent cell, such as an iPSC, but may be or have one or more characteristics of a multipotent cell, a proliferating cell and/or a Müller glia progenitor cell. Alternatively, the transcription factors disclosed herein may directly convert a macroglial source cell to a cone-like photoreceptor cell and/or to an RPE-like cell.

The present disclosure provides a method for cell conversion of a Müller glia cell to a cone photoreceptor cell by introducing at least one or more transcription factors, or variants thereof, selected from the group consisting of MEF2C, MEF2D and RXRG, and optionally CRX, or any combination thereof, into the Müller Glia cells thereby generating a cone photoreceptor. The Muller glia cells are cultured for a sufficient time and under conditions to allow direct conversion to photoreceptor cells, wherein at least 0.01% of Müller glia cells are converted to a photoreceptor cell. In some aspects, at least 0.01%, at least 0.02%, at least 0.05%, at least 0.075% or at least 0.1% of Müller glia cells are converted to photoreceptor cells. In other aspects, at least 0.1%, at least 0.5%, at least 1%, at least 5% or at least 10% of Müller glia cells are converted to photoreceptor cells. In further aspects, at least 10% to 20%, preferably over 50%, more preferably at least 85%, 95% or 100% of the Müller glia cells are converted to a photoreceptor cell. By “converted”, it is meant that there is an up-regulation of any one or more cell markers of a cone photoreceptor, or a down-regulation of one or more cell markers of a source cell (e.g. one or more markers of a Müller glia, or an astrocyte cell), and/or one or more change in cell morphology from the source to a cone photoreceptor, or any combination thereof. Changes in morphology can be identified by observing at microscopic level. For example, when converting from a Müller glia cell to a cone photoreceptor, morphological changes include a change from an elongated cell that transverses all layers of the retina to a photoreceptor shaped cell in the ONL with a cone shaped outer segment. Up-regulation of markers can be observed by staining cells for specific markers or by analysing the expression of markers at the gene level or by undertaking single-cell RNA sequencing. For example, when converting from a Müller glia cell to a cone photoreceptor, up-regulation of at least one marker includes up-regulation of cone arrestin, a specific cone marker.

Up-regulation of a cone cell marker, down-regulation of a source cell marker and/or changes in cell morphology are all considered to be characteristics of a cone photoreceptor cell produced by methods according to the present invention. In some aspects, at least 0.01%, at least 0.05%, at least 0.1%, at least 0.5%, at least 1%, at least 5% or at least 10% of the converted cone photoreceptors exhibit at least one characteristic of the cone photoreceptors.

The present disclosure provides a method for cell conversion of a Müller glia cell to an RPE-like cell by introducing at least one or more transcription factors, or variants thereof, comprising MEF2C into the Müller Glia cells thereby generating an RPE-like cell. The one or more transcription factors may further comprise one or more of MEF2D and RXRG. The one or more transcription factors may further comprise CRX. The Muller glia cells are cultured for a sufficient time and under conditions to allow direct conversion to RPE-like cells, wherein at least 0.01% of Müller glia cells are converted to an RPE-like cell. In some aspects, at least 0.01%, at least 0.02%, at least 0.05%, at least 0.075% or at least 0.1% of Müller glia cells are converted to RPE-like cells. In other aspects, at least 0.1%, at least 0.5%, at least 1%, at least 5% or at least 10% of Muller glia cells are converted to RPE-like cells. In further aspects, at least 10% to 20%, preferably over 50%, more preferably at least 85%, 95% or 100% of the Müller glia cells are converted to RPE-like cells. By “converted”, it is meant that there is an up-regulation of any one or more cell markers of an RPE-like cell, or a down-regulation of one or more cell markers of a source cell (e.g. one or more markers of a Muller glia, or an astrocyte cell), and/or one or more change in cell morphology from the source to an RPE-like cell, or any combination thereof. Changes in morphology can be identified by observing at microscopic level. For example, when converting from a Müller glia cell to an RPE-like cell, morphological changes include a change from an elongated cell that transverses all layers of the retina to a pigmented cell. Up-regulation of markers can be observed by staining cells for specific markers or by analysing the expression of markers at the gene level or by undertaking single-cell RNA sequencing. For example, when converting from a Müller glia cell to an RPE-like cell, up-regulation of at least one marker includes up-regulation of BEST1, a specific RPE cell marker.

Up-regulation of an RPE cell marker, down-regulation of a source cell marker and/or changes in cell morphology are all considered to be characteristics of an RPE-like cell produced by methods according to the present invention. In some aspects, at least 0.01%, at least 0.05%, at least 0.1%, at least 0.5%, at least 1%, at least 5% or at least 10% of the converted RPE-like cells exhibit at least one characteristic of the RPE-like cells.

The present disclosure provides a method of converting a source cell selected from a Müller glia or an astrocyte cell to a cone photoreceptor cell, by introducing one or more nucleic acids thereby increasing the expression of one or more transcription factors, or variants thereof, selected from MEF2C, MEF2D and RXRG or any combination thereof in the source cell. The one or more transcription factors can comprise at least one, at least two, or at least three of MEF2C, MEF2D and RXRG in any combination thereof. In one embodiment, the one or more transcription factor may be one transcription factor alone, for example; MEF2C or MEF2D or RXRG alone. In other embodiments, one or more transcription factors may be a combination of two transcription factors in any combination, for example; MEF2C and MEF2D, or MEF2C and RXRG, or MEF2D and RXRG. In other embodiments, the one or more transcription factors may be a combination of three transcription factors, for example, MEF2C, MEF2D and RXRG. In other embodiments, the one or more nucleic acid further encodes CRX. In one embodiment, the one or more nucleic acid encodes MEF2C, MEF2D, RXRG and CRX thereby increasing the expression of all four transcription factors in the source cell.

The present disclosure provides a method for converting a source cell selected from: a Müller glia cell or an astrocyte to a cone photoreceptor cell, the method comprising:
i) providing a source cell, or a cell population comprising a source cell;
ii) introducing one or more nucleic acids encoding at least one or more transcription factors, or variants thereof, selected from the group consisting of MEF2C, MEF2D, RXRG and CRX into the source cell thereby increasing the expression of the at least one or more transcription factors;
iii) culturing said source cell or cell population comprising a source cell until at least one characteristic of a cone photoreceptor cell is observed.

The present disclosure provides a method for converting a source cell selected from: a Müller glia cell or an astrocyte to a cone-like photoreceptor cell, the method comprising:
i) providing a source cell, or a cell population comprising a source cell;
ii) introducing one or more nucleic acids encoding at least one or more transcription factors, or variants thereof, comprising MEF2C (and optionally further comprising MEF2D and/or RXRG and/or CRX) into the source cell thereby increasing the expression of the at least one or more transcription factors;
iii) culturing said source cell or cell population comprising a source cell until at least one characteristic of a cone-like photoreceptor cell is observed.

The present disclosure provides a method for converting a source cell selected from: a Müller glia cell or an astrocyte to an RPE-like cell, the method comprising:
i) providing a source cell, or a cell population comprising a source cell;
ii) introducing one or more nucleic acids encoding at least one or more transcription factors, or variants thereof, comprising MEF2C (and optionally further comprising MEF2D and/or RXRG and/or CRX) into the source cell thereby increasing the expression of the at least one or more transcription factors;
iii) culturing said source cell or cell population comprising a source cell until at least one characteristic of an RPE-like cell is observed.

A person skilled in the art would routinely culture cells and therefore be familiar with using appropriate medium and culture conditions to support Müller glia cell development and proliferation. The source cells e.g. Müller glia cells and astrocytes can be cultured according to standard protocols to generate retinal organoids from iPSCs and retinal explant cultures, while astrocytes are present in brain organoids and retinal explants only as they are not derived from retinal progenitor cells like Müller glia. The transduced cells can be monitored until at least one characteristic of a cone photoreceptor cell and/or RPE cell is observed, for example, using a fluorescent protein as part of the viral vector, such as GFP. The time in culture before at least one characteristic of a cone photoreceptor cell and/or RPE cell is observed may vary according to experimental system and vector choice. For example, less time may be needed when a repRNA vector is used compared to a viral vector such as lentivirus or AAV. And less time may be needed in a 2D Müller glia culture than in a 3D retinal organoid. When the cells are 2D Müller glia and the vector is repRNA, the cells may be cultured for around 2 days to around 7 days (for example at least 2, at least 3, at least 4, at least 5 or at least 6 days). When the cells are 2D Müller glia and the vector is a viral vector such as lentivirus or AAV, the cells may be cultured for around 7 days to around 14 days (for example at least 7,at least 8, at least 9, at least 10, at least 11, at least 12 or at least 13 days). When the cells are retinal organoids, the cells may be cultured for around 7 days to around 28 days (for example for example at least 7, at least 8, at least 9, at least 10, at least 11, at least 12, at least 13, at least 14, at least 15, at least 16,at least 17, at least 18, at least 19, at least 20, at least 21, at least 22, at least 23, at least 24, at least 25,at least 26 or at least 27 days. When the cells are retinal explants, such as mouse retinal explants, the cells may be cultured for around 10 days to around 14 days (for example for example at least 10, at least 11, at least 12 or at least 13 days), Suitable media for culturing the source cells such as Müller glia cells and astrocytes are known to those skilled in the art. Examples of suitable medium include B27-and N2-based retinal differentiation medium. Once retinal organoids have been generated from iPSCs, the source cells may be cultured for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, preferably the source cells are cultured for between 14 and 21 days post-transduction. The length of the culture time may be the overall culture time i.e. prior to transducing the cells, and until cells have transdifferentiated to a cone photoreceptor, or it may be the culture time post-transduction of the source cells.

Small molecules may be used to activate Müller glia cells and induce GFAP expression, therefore, increase their proliferation. An example of a small molecule capable of increasing activation and proliferation of Müller glia cells is purmorphamine. Activation of Müller glia is required in human retinal organoids as they are ‘healthy’ and do not exhibit GFAP expression as they are in development and are not diseased. Purmorphamine will not be required to treat patients with retinal degeneration, as Müller glia are activated and express GFAP in retinal disease and trauma. The method may therefore comprise contacting the cells with puromorphamine. The method may therefore comprise contacting the cells with puromorphamine wherein one or more transcription factor is under the control of a GFAP promoter.

As used herein a “transcription factor” refers to a protein whose function is to regulate the expression of a particular gene or genes in a cell and controls the rate of transcription of said gene's DNA to messenger RNA.

As used herein, “MEF2C” refers to Myocyte Enhancer Factor 2C. MEF2C is also known as Myocyte-specific Enhancer Factor 2D or MADS Box Transcription Enhancer Factor 2, Polypeptide C. MEF2C belongs to the MEF2 family of transcription factors, which play a role in myogenesis. MEF2A is known to be an important paralog of the MEF2C gene. The Ensembl gene ID is ENSG00000081189.16. An example of a transcript is the Ensembl transcript ID ENST00000636998.1 and the UniParc ID is Q06413-3, codon-optimised and alternatively spliced transcript variants are encompassed. MEF2C is a transcription factor, which has been shown to directly reprogram cardiac fibroblasts into Cardiomyocytes.

MEF2C may have the following amino acid sequence, identified as SEQ ID NO: 1

SEQ ID NO: 1
MGRKKIQITRIMDERNRQVTFTKRKFGLMKKAYELSVLCDCEIAL
IIFNSTNKLFQYASTDMDKVLLKYTEYNEPHESRTNSDIVETLRK
KGLNGCDSPDPDADDSVGHSPESEDKYRKINEDIDLMISRQRLCA
VPPPNFEMPVSIPVSSHNSLVYSNPVSSLGNPNLLPLAHPSLQRN
SMSPGVTHRPPSAGNTGGLMGGDLTSGAGTSAGNGYGNPRNSPGL
LVSPGNLNKNMQAKSPPPMNLGMNNRKPDLRVLIPPGSKNTMPSV
SEDVDLLLNQRINNSQSAQSLATPVVSVATPTLPGQGMGGYPSAI
STTYGTEYSLSSADLSSLSGFNTASALHLGSVTGWQQQHLHNMPP
SALSQLGDRTTTPSRYPQHTRHEAGRSPVDSLSSCSSSYDGSDRE
DHRNEFHSPIGLTRPSPDERESPSVKRMRLSEGWAT

MEF2C may be encoded by the following “wild-type” sequence, identified as SEQ ID NO: 2

SEQ ID NO: 2
ATGGGGAGAAAAAAGATTCAGATTACGAGGATTATGGATGAACGT
AACAGACAGGTGACATTTACAAAGAGGAAATTTGGGTTGATGAAG
AAGGCTTATGAGCTGAGCGTGCTGTGTGACTGTGAGATTGCGCTG
ATCATCTTCAACAGCACCAACAAGCTGTTCCAGTATGCCAGCACC
GACATGGACAAAGTGCTTCTCAAGTACACGGAGTACAACGAGCCG
CATGAGAGCCGGACAAACTCAGACATCGTGGAGACGTTGAGAAAG
AAGGGCCTTAATGGCTGTGACAGCCCAGACCCCGATGCGGACGAT
TCCGTAGGTCACAGCCCTGAGTCTGAGGACAAGTACAGGAAAATT
AACGAAGATATTGATCTAATGATCAGCAGGCAAAGATTGTGTGCT
GTTCCACCTCCCAACTTCGAGATGCCAGTCTCCATCCCAGTGTCC
AGCCACAACAGTTTGGTGTACAGCAACCCTGTCAGCTCACTGGGA
AACCCCAACCTATTGCCACTGGCTCACCCTTCTCTGCAGAGGAAT
AGTATGTCTCCTGGTGTAACACATCGACCTCCAAGTGCAGGTAAC
ACAGGTGGTCTGATGGGTGGAGACCTCACGTCTGGTGCAGGCACC
AGTGCAGGGAACGGGTATGGCAATCCCCGAAACTCACCAGGTCTG
CTGGTCTCACCTGGTAACTTGAACAAGAATATGCAAGCAAAATCT
CCTCCCCCAATGAATTTAGGAATGAATAACCGTAAACCAGATCTC
CGAGTTCTTATTCCACCAGGCAGCAAGAATACGATGCCATCAGTG
TCTGAGGATGTCGACCTGCTTTTGAATCAAAGGATAAATAACTCC
CAGTCGGCTCAGTCATTGGCTACCCCAGTGGTTTCCGTAGCAACT
CCTACTTTACCAGGACAAGGAATGGGAGGATATCCATCAGCCATT
TCAACAACATATGGTACCGAGTACTCTCTGAGTAGTGCAGACCTG
TCATCTCTGTCTGGGTTTAACACCGCCAGCGCTCTTCACCTTGGT
TCAGTAACTGGCTGGCAACAGCAACACCTACATAACATGCCACCA
TCTGCCCTCAGTCAGTTGGGAGACCGTACCACCACCCCTTCGAGA
TACCCACAACACACGCGCCACGAGGCGGGGAGATCTCCTGTTGAC
AGCTTGAGCAGCTGTAGCAGTTCGTACGACGGGAGCGACCGAGAG
GATCACCGGAACGAATTCCACTCCCCCATTGGACTCACCAGACCT
TCGCCGGACGAAAGGGAAAGTCCCTCAGTCAAGCGCATGCGACTT
TCTGAAGGATGGGCAACATGA

MEF2C may be encoded by the following “codon-optimised” sequence, identified as SEQ ID NO: 3

SEQ ID NO: 3
atgggaagaaagaaaatccaaataaccagaatcatggacgagcgg
aataggcaagttaccttcacgaaacggaagttcggactgatgaag
aaggcctacgaactcagtgtcttgtgcgattgcgagatcgccctt
attatctttaattctacaaacaaactcttccaatacgcttccact
gatatggataaggtgttgctgaagtacaccgagtataatgaacca
cacgaaagtcgtaccaattctgatattgtagaaaccctcaggaag
aaaggactcaacgggtgcgatagtcctgatccggacgctgatgac
tctgttgggcattctcccgaatcagaagataaatatagaaagatc
aatgaggacatcgacctgatgatttcccggcagaggctctgcgcg
gtcccgccccctaattttgaaatgcccgtgagtattcctgtctct
agtcataatagcctggtctattccaatccagtgtcaagccttggg
aatccaaatctgctgcctctcgcccatccctcattgcaacggaac
tcaatgagtcccggcgtcacccacagaccgccatccgccggaaat
actggcggccttatgggcggtgatctgacatcaggcgctgggact
tcagccggaaatggatacggaaacccacgcaatagtccgggcttg
cttgtgtctccaggaaatcttaataagaacatgcaggctaagagt
ccaccacctatgaacctgggcatgaacaataggaagcccgacttg
agggtgctcatcccacccggttctaagaacaccatgccttccgtt
tcagaagacgtggatctcttgctcaaccagcgaatcaacaattct
caaagcgcccaatctcttgcaacgcctgtagtctctgttgctaca
cccacactgcctgggcaggggatgggcggctaccctagcgctatc
tccactacgtacggcactgaatattcccttagcagcgccgatctc
agcagtttgagcggcttcaatacagcttccgcgctgcatctgggg
tctgttactggttggcagcaacagcatttgcacaatatgccgcct
agcgcgcttagccaactcggggatcggactacaacaccctctcgc
tatccgcagcatactcggcatgaagcaggacgatcccccgtggat
tcactgtctagttgctcctcttcctatgatggcagtgatagggaa
gaccatcgtaatgagtttcatagtccaatcggcctgacacggcca
tcacctgatgagcgcgagtctccttctgtgaaacggatgcggctg
tcagaggggtgggctacctaa

As used herein, “MEF2D” refers to Myocyte Enhancer Factor 2D. MEF2D is also known as Myocyte-specific Enhancer Factor 2D or MADS Box Transcription Enhancer Factor 2, Polypeptide D. MEF2D belongs to the MEF2 family of transcription factors. MEF2A is known to be an important paralog of the MEF2D gene. The

Ensembl gene ID is ENSG00000116604.19. An example of a transcript is the Ensembl transcript ID ENST00000348159.9 and the UniParc ID is Q14814-1, codon-optimised and alternatively spliced transcript variants are encompassed. MEF2D is a transcription factor, which is known to mediate cellular functions in skeletal and cardiac muscle development, as well as neuronal differentiation and survival, with a critical role in the regulation of neuronal apoptosis.

MEF2D may have the following amino acid sequence, identified as SEQ ID NO: 4

SEQ ID NO: 4
MGRKKIQIQRITDERNRQVTFTKRKFGLMKKAYELSVLCDCEIAL
IIFNHSNKLFQYASTDMDKVLLKYTEYNEPHESRTNADIIETLRK
KGFNGCDSPEPDGEDSLEQSPLLEDKYRRASEELDGLFRRYGSTV
PAPNFAMPVTVPVSNQSSLQFSNPSGSLVTPSLVTSSLTDPRLLS
PQQPALQRNSVSPGLPQRPASAGAMLGGDLNSANGACPSPVGNGY
VSARASPGLLPVANGNSLNKVIPAKSPPPPTHSTQLGAPSRKPDL
RVITSQAGKGLMHHLTEDHLDLNNAQRLGVSQSTHSLTTPVVSVA
TPSLLSQGLPFSSMPTAYNTDYQLTSAELSSLPAFSSPGGLSLGN
VTAWQQPQQPQQPQQPQPPQQQPPQPQQPQPQQPQQPQQPPQQQS
HLVPVSLSNLIPGSPLPHVGAALTVTTHPHISIKSEPVSPSRERS
PAPPPPAVFPAARPEPGDGLSSPAGGSYETGDRDDGRGDFGPTLG
LLRPAPEPEAEGSAVKRMRLDTWTLK

MEF2D may be encoded by the following “wild-type” sequence, identified as SEQ ID NO: 5

SEQ ID NO: 5
ATGGGGAGGAAAAAGATTCAGATCCAGCGAATCACCGACGAGCGG
AACCGACAGGTGACTTTCACCAAGCGGAAGTTTGGCCTGATGAAG
AAGGCGTATGAGCTGAGCGTGCTATGTGACTGCGAGATCGCACTC
ATCATCTTCAACCACTCCAACAAGCTGTTCCAGTACGCCAGCACC
GACATGGACAAGGTGCTGCTCAAGTACACGGAGTACAATGAGCCA
CACGAGAGCCGCACCAACGCCGACATCATCGAGACCCTGAGGAAG
AAGGGCTTCAACGGCTGCGACAGCCCCGAGCCCGACGGGGAGGAC
TCGCTGGAACAGAGCCCCCTGCTGGAGGACAAGTACCGACGCGCC
AGCGAGGAGCTCGACGGGCTCTTCCGGCGCTATGGGTCAACTGTC
CCGGCCCCCAACTTTGCCATGCCTGTCACGGTGCCCGTGTCCAAT
CAGAGCTCACTGCAGTTCAGCAATCCCAGCGGCTCCCTGGTCACC
CCTTCCCTGGTGACATCATCCCTCACGGACCCGCGGCTCCTGTCC
CCCCAGCAGCCAGCACTACAGAGGAACAGTGTGTCTCCTGGCCTG
CCCCAGCGGCCAGCTAGTGCGGGGGCCATGCTGGGGGGTGACCTG
AACAGTGCTAACGGAGCCTGCCCCAGCCCTGTTGGGAATGGCTAC
GTCAGTGCTCGGGCTTCCCCTGGCCTCCTCCCTGTGGCCAATGGC
AACAGCCTAAACAAGGTCATCCCTGCCAAGTCTCCACCCCCACCT
ACCCACAGCACCCAGCTTGGAGCCCCCAGCCGCAAGCCCGACCTG
CGAGTCATCACTTCCCAGGCAGGAAAGGGGTTAATGCATCACTTG
ACTGAGGACCATTTAGATCTGAACAATGCCCAGCGCCTTGGGGTC
TCCCAGTCTACTCATTCGCTCACCACCCCAGTGGTTTCTGTGGCA
ACGCCGAGTTTACTCAGCCAGGGCCTCCCCTTCTCTTCCATGCCC
ACTGCCTACAACACAGATTACCAGTTGACCAGTGCAGAGCTCTCC
TCCTTACCAGCCTTTAGTTCACCTGGGGGGCTGTCGCTAGGCAAT
GTCACTGCCTGGCAACAGCCACAGCAGCCCCAGCAGCCGCAGCAG
CCACAGCCTCCACAGCAGCAGCCACCGCAGCCACAGCAGCCACAG
CCACAGCAGCCTCAGCAGCCGCAACAGCCACCTCAGCAACAGTCC
CACCTGGTCCCTGTATCTCTCAGCAACCTCATCCCGGGCAGCCCC
CTGCCCCACGTGGGTGCTGCCCTCACAGTCACCACCCACCCCCAC
ATCAGCATCAAGTCAGAACCGGTGTCCCCAAGCCGTGAGCGCAGC
CCTGCGCCTCCCCCTCCAGCTGTGTTCCCAGCTGCCCGCCCTGAG
CCTGGCGATGGTCTCAGCAGCCCAGCCGGGGGATCCTATGAGACG
GGAGACCGGGATGACGGACGGGGGGACTTCGGGCCCACACTGGGC
CTGCTGCGCCCAGCCCCAGAGCCTGAGGCTGAGGGCTCAGCTGTG
AAGAGGATGCGGCTTGATACCTGGACATTAAAGTGA

MEF2D may be encoded by the following “codon-optimised” sequence, identified as SEQ ID NO: 6

SEQ ID NO: 6
atgggaagaaagaaaatccaaatacaaagaattacagatgaacgc
aacagacaagttacattcaccaaaaggaagttcggtttgatgaag
aaagcctacgaactttcagtcctctgcgattgtgaaatagccctg
attatcttcaatcatagcaacaaactgttccaatatgccagcaca
gatatggataaagtactcctgaagtatacagaatataacgaaccc
cacgaatcacggacaaatgctgatattattgaaacgcttcgcaag
aaagggtttaatgggtgtgatagtcctgaaccagatggcgaagat
agcttggagcaatccccattgctcgaagataaatatcgcagagct
tctgaagaattggatggactgtttagacggtacggctccaccgtg
cctgctccaaatttcgctatgccagttacagttccagtttctaac
caatcaagcctgcaattttctaacccttctggtagtctcgtgaca
ccctctctcgttacgtctagcttgacagatcctaggctgttgtct
cctcaacaacctgccctgcaacggaattcagtctcaccgggactc
ccacaacgtcccgcaagcgcaggagcgatgctcggcggggatctt
aatagcgcgaatggcgcttgtccttcacccgtaggcaacgggtat
gtgtcagcgcgcgcaagccccggactgctgcccgttgcgaacggt
aattcactgaacaaagtgattcccgcaaagagtcctcctccacca
acacattctacacaactgggcgcacctagtagaaaacctgatttg
cgcgtgattacctctcaagccggcaaaggcctgatgcaccatctc
acggaagatcaccttgacctcaataacgcacaaagactcggagtg
agccaatccacacactccctgacgacgcccgtcgtctccgtagcc
accccatcccttttgtcacaaggtctgccatttagctctatgcct
acagcttataatactgactatcaactgacgtccgctgaactgagt
tcacttcctgctttctccagccccggtggcctctcacttgggaac
gtgaccgcgtggcagcaacctcaacaaccacaacaaccccaacaa
ccgcaaccgcctcaacaacaaccccctcaaccgcaacaaccccaa
ccccaacaaccgcaacaaccccagcaaccgccccaacagcaaagc
catcttgttccagtgagtctgtccaatctgattcctggaagccca
ctccctcatgttggcgcggctctgactgtgactactcatcctcat
atttctatcaaaagtgagcctgtttctccctctagagaaagatct
cccgctccgcccccacccgccgtctttcctgccgctagacccgaa
ccaggggacgggctgtcctcacctgcaggcggaagttacgaaacc
ggggatagagacgatggtagaggtgattttggccctactcttgga
ctgctccgacctgcaccggaacccgaagccgaaggaagcgccgta
aaacgaatgagactggacacatggacgcttaaataa

As used herein, “RXRG” refers to Retinoid X Receptor Gamma. RXRG is also known as Retinoic Acid Receptor RXR-Gamma, Retinoid X Receptor Gamma, NR2B3, and Nuclear Receptor Subfamily 2 Group

B Member 3. The Ensembl gene ID is ENSG00000143171.13. An example of a transcript is the Ensembl transcript ID ENST00000359842.10 and the UniParc ID is P48443-1, codon-optimised and alternatively spliced transcript variants are encompassed. RXRG is a transcription factor, which belongs to the retinoid X receptor (RXR) family of nuclear receptors, known to be involved in mediating the antiproliferative effects of retinoic acid (RA). There are 3 subtypes of the RXR gene, alpha (RXRA), beta (RXRB) and gamma

(RXRG), RXRA is known to be an important paralog of RXRG.

RXRG may have the following amino acid sequence, identified as SEQ ID NO: 7

SEQ ID NO: 7
MYGNYSHFMKFPAGYGGSPGHTGSTSMSPSAALSTGKPMDSHPSY
TDTPVSAPRTLSAVGTPLNALGSPYRVITSAMGPPSGALAAPPGI
NLVAPPSSQLNVVNSVSSSEDIKPLPGLPGIGNMNYPSTSPGSLV
KHICAICGDRSSGKHYGVYSCEGCKGFFKRTIRKDLIYTCRDNKD
CLIDKRQRNRCQYCRYQKCLVMGMKREAVQEERQRSRERAESEAE
CATSGHEDMPVERILEAELAVEPKTESYGDMNMENSTNDPVTNIC
HAADKQLFTLVEWAKRIPHFSDLTLEDQVILLRAGWNELLIASFS
HRSVSVQDGILLATGLHVHRSSAHSAGVGSIFDRVLTELVSKMKD
MQMDKSELGCLRAIVLFNPDAKGLSNPSEVETLREKVYATLEAYT
KQKYPEQPGRFAKLLLRLPALRSIGLKCLEHLFFFKLIGDTPIDT
FLMEMLETPLQIT

RXRG may be encoded by the following “wild-type” sequence, identified as SEQ ID NO: 8

SEQ ID NO: 8
ATGTATGGAAATTATTCTCACTTCATGAAGTTTCCCGCAGGCTAT
GGAGGCTCCCCTGGCCACACTGGCTCTACATCCATGAGCCCATCA
GCAGCCTTGTCCACAGGGAAGCCAATGGACAGCCACCCCAGCTAC
ACAGATACCCCAGTGAGTGCCCCACGGACTCTGAGTGCAGTGGGG
ACCCCCCTCAATGCCCTGGGCTCTCCATATCGAGTCATCACCTCT
GCCATGGGCCCACCCTCAGGAGCACTTGCAGCGCCTCCAGGAATC
AACTTGGTTGCCCCACCCAGCTCTCAGCTAAATGTGGTCAACAGT
GTCAGCAGTTCAGAGGACATCAAGCCCTTACCAGGGCTTCCCGGG
ATTGGAAACATGAACTACCCATCCACCAGCCCCGGATCTCTGGTT
AAACACATCTGTGCCATCTGTGGAGACAGATCCTCAGGAAAGCAC
TACGGGGTATACAGTTGTGAAGGCTGCAAAGGGTTCTTCAAGAGG
ACGATAAGGAAGGACCTCATCTACACGTGTCGGGATAATAAAGAC
TGCCTCATTGACAAGCGTCAGCGCAACCGCTGCCAGTACTGTCGC
TATCAGAAGTGCCTTGTCATGGGCATGAAGAGGGAAGCTGTGCAA
GAAGAAAGACAGAGGAGCCGAGAGCGAGCTGAGAGTGAGGCAGAA
TGTGCTACCAGTGGTCATGAAGACATGCCTGTGGAGAGGATTCTA
GAAGCTGAACTTGCTGTTGAACCAAAGACAGAATCCTATGGTGAC
ATGAATATGGAGAACTCGACAAATGACCCTGTTACCAACATATGT
CATGCTGCTGACAAGCAGCTTTTCACCCTCGTTGAATGGGCCAAG
CGTATTCCCCACTTCTCTGACCTCACCTTGGAGGACCAGGTCATT
TTGCTTCGGGCAGGGTGGAATGAATTGCTGATTGCCTCTTTCTCC
CACCGCTCAGTTTCCGTGCAGGATGGCATCCTTCTGGCCACGGGT
TTACATGTCCACCGGAGCAGTGCCCACAGTGCTGGGGTCGGCTCC
ATCTTTGACAGAGTCCTAACTGAGCTGGTTTCCAAAATGAAAGAC
ATGCAGATGGACAAGTCGGAACTGGGATGCCTGCGAGCCATTGTA
CTCTTTAACCCAGATGCCAAGGGCCTGTCCAACCCCTCTGAGGTG
GAGACTCTGCGAGAGAAGGTTTATGCCACCCTTGAGGCCTACACC
AAGCAGAAGTATCCGGAACAGCCAGGCAGGTTTGCCAAGCTGCTG
CTGCGCCTCCCAGCTCTGCGTTCCATTGGCTTGAAATGCCTGGAG
CACCTCTTCTTCTTCAAGCTCATCGGGGACACCCCCATTGACACC
TTCCTCATGGAGATGTTGGAGACCCCGCTGCAGATCACCTGA

RXRG may be encoded by the following “codon-optimised” sequence, identified as SEQ ID NO: 9

SEQ ID NO: 9
atgtacggcaactactcacattttatgaaattcccagccggatac
gggggtctcccggccatacaggtagtacgagcatgtcccccagcg
cggcgctgagcaccggcaaacccatggattcccatccttcttata
ccgacacgcctgtcagcgctcccagaacactctctgccgtcggca
cgcctctgaacgctcttggtagtccctacagagttattacatccg
ctatgggaccaccgagtggggcgctggctgccccacccggcataa
atctcgtggctccgccaagctcccaattgaacgtcgtgaattctg
tatcttctagtgaagacataaaacctcttcctggcctgccgggta
tcggtaatatgaattatccttctacgtcccctggttcactcgtga
agcatatttgcgctatttgcggcgatcggtcatctgggaaacatt
atggagtctattcctgcgagggatgtaagggtttctttaaacgta
caatccgtaaagatcttatttatacctgcagggacaacaaggatt
gtcttatcgacaaaaggcaacgaaatcggtgtcaatattgcagat
accagaaatgtctcgtgatgggtatgaaaagagaggccgtccagg
aagagcgccaacggagtagagaaagggcggaatccgaagctgagt
gcgccactagcgggcacgaggatatgcccgtcgaacgtatccttg
aggcagagctggcggtcgagcccaaaactgagagctacggggata
tgaacatggaaaattccactaacgatccagtcactaatatctgcc
acgcagccgataaacaactgtttacattggtcgagtgggctaaac
gcataccacattttagtgatctgacacttgaagatcaagttatcc
ttctccgagctggctggaacgagcttctcatagcatccttttctc
atagatctgtgtctgtccaagacgggattctcctcgcaacaggcc
tgcacgtgcataggtccagcgcgcatagcgcaggcgtggggagta
ttttcgatagggtgttgaccgaactcgtgagcaagatgaaggata
tgcaaatggataaatcagagttgggctgtctccgcgcaatagtgt
tgttcaatcccgacgctaaagggctcagcaatcctagtgaagtcg
aaaccttgagggaaaaggtatacgctacattggaagcgtatacca
aacaaaagtaccccgagcaacctgggcgattcgcaaaacttcttt
tgcggctgcctgccctgcggagtatcgggcttaagtgtttggaac
atctgttcttctttaaactgataggtgatactccgatcgacacat
ttctgatggaaatgctcgaaacgcctctccaaattacataa

As used herein, “CRX” refers to Cone-Rod Homeobox. CRX is also known as CORD2, LCA7, OTX3, CRD or Orthodenticle Homeobox 3. CRX is considered to be part of the OTX family of genes. The Ensembl gene

ID is ENSG00000105392.16. An example of a transcript is the Ensembl transcript ID ENST00000221996.12 and the UniParc ID is 043186-1, codon-optimised and alternatively spliced transcript variants are encompassed. The protein encoded by the CRX gene is a photoreceptor-specific transcription factor which plays a role in the differentiation of photoreceptor cells, being necessary for the maintenance of normal cone and rod function. The CRX gene acts synergistically with other transcription factors, such as, NRL, RORB and RAX, to regulate photoreceptor cell-specific gene transcription. OTX2 is known to be an important paralog of the CRX gene and may be used interchangeably with CRX as a transcription factor according to the present invention.

CRX may have the following amino acid sequence, identified as SEQ ID NO: 10

SEQ ID NO: 10
MMAYMNPGPHYSVNALALSGPSVDLMHQAVPYPSAPRKQRRERTT
FTRSQLEELEALFAKTQYPDVYAREEVALKINLPESRVQVWFKNR
RAKCRQQRQQQKQQQQPPGGQAKARPAKRKAGTSPRPSTDVCPDP
LGISDSYSPPLPGPSGSPTTAVATVSIWSPASESPLPEAQRAGLV
ASGPSLTSAPYAMTYAPASAFCSSPSAYGSPSSYFSGLDPYLSPM
VPQLGGPALSPLSGPSVGPSLAQSPTSLSGQSYGAYSPVDSLEFK
DPTGTWKFTYNPMDPLDYKDQSAWKFQIL

CRX may be encoded by the following “wild-type” sequence, identified as SEQ ID NO: 11

SEQ ID NO: 11
ATGATGGCGTATATGAACCCGGGGCCCCACTATTCTGTCAACGCC
TTGGCCCTAAGTGGCCCCAGTGTGGATCTGATGCACCAGGCTGTG
CCCTACCCAAGCGCCCCCAGGAAGCAGCGGGGGAGCGCACCACCT
TCACCCGGAGCCAACTGGAGGAGCTGGAGGCACTGTTTGCCAAGA
CCCAGTACCCAGACGTCTATGCCCGTGAGGAGGTGGCTCTGAAGA
TCAATCTGCCTGAGTCCAGGGTTCAGGTTTGGTTCAAGAACCGGA
GGGCTAAATGCAGGCAGCAGCGACAGCAGCAGAAACAGCAGCAGC
AGCCCCCAGGGGGCCAGGCCAAGGCCCGGCCTGCCAAGAGGAAGG
CGGGCACGTCCCCAAGACCCTCCACAGATGTGTGTCCAGACCCTC
TGGGCATCTCAGATTCCTACAGTCCCCCTCTGCCCGGCCCCTCAG
GCTCCCCAACCACGGCAGTGGCCACTGTGTCCATCTGGAGCCCAG
CCTCAGAGTCCCCTTTGCCTGAGGCGCAGCGGGCTGGGCTGGTGG
CCTCAGGGCCGTCTCTGACCTCCGCCCCCTATGCCATGACCTACG
CCCCGGCCTCCGCTTTCTGCTCTTCCCCCTCCGCCTATGGGTCTC
CGAGCTCCTATTTCAGCGGCCTAGACCCCTACCTTTCTCCCATGG
TGCCCCAGCTAGGGGGCCCGGCTCTTAGCCCCCTCTCTGGCCCCT
CCGTGGGACCTTCCCTGGCCCAGTCCCCCACCTCCCTATCAGGCC
AGAGCTATGGCGCCTACAGCCCCGTGGATAGCTTGGAATTCAAGG
ACCCCACGGGCACCTGGAAATTCACCTACAATCCCATGGACCCTC
TGGACTACAAGGATCAGAGTGCCTGGAAGTTTCAGATCTTGTAG

CRX may be encoded by the following “codon-optimised” sequence, identified as SEQ ID NO: 12

SEQ ID NO: 12
atgatggcctacatgaatcctggtccacattactcagtaaatgct
ctcgcacttagcggtcctagcgtagacctcatgcatcaagcggtc
ccgtatccctcagcgccgcggaaacaaagacgtgaaaggacaaca
tttactagatctcagcttgaagaactcgaagccttgttcgcgaaa
acacaatatcccgatgtgtacgcacgggaagaagtcgccctcaaa
ataaacttgccagaatctcgtgtacaagtgtggtttaagaataga
cgcgccaagtgtagacaacaaagacaacaacaaaagcaacaacaa
caaccgccgggcggtcaagctaaagctaggcccgctaaacggaaa
gctggtacaagcccccggccgtcaactgacgtttgccccgatcca
cttggtatttctgactcttattccccgcccttgcctggaccgagc
ggtagtcccacaaccgctgtcgcaacggtatctatttggtccccg
gccagcgaatctcccctgccagaagcacaacgcgcaggcctcgtc
gcatccggcccttcactcactagcgcaccatacgcaatgacgtat
gcaccagctagtgcattttgttcctcaccttctgcatacggcagc
ccctcatcatacttttctgggctggacccttatctgtcacctatg
gtcccgcaattgggcggacctgccctgagtccacttagcggaccg
agcgtcggtccgtcactcgctcaaagcccgacatctctcagcggt
caatcttacggagcatattcacctgtcgactcccttgagtttaaa
gatccaactgggacatggaagtttacttataacccaatggaccca
ctcgattataaagaccaatccgcgtggaaattccaaattctctga

As used herein, “OTX2” refers to Orthodenticle Homeobox 2. OTX2 is also considered to be part of the OTX family of genes. The Ensembl gene ID is ENSG00000165588.19. An example of a transcript is the Ensembl transcript ID ENST00000672264.2 and the UniParc ID is P32243-1, codon-optimised and alternatively spliced transcript variants are encompassed. The protein encoded by the OTX2 gene is a transcription factor, which plays a broad role in cone photoreceptor cell development and function. The single Drosophila orthodenticle (otd) gene is represented by three vertebrate OTX factors, OTX1, OTX2,and CRX. OTX2 is well established as a key regulator of retinal development and is essential for retinal pigment epithelium and photoreceptor differentiation, whereas OTX1 may be important for ciliary body development. CRX is a photoreceptor specific gene that is known to be essential for photoreceptor development.

“Engineered” or “non-naturally occurring” are terms that indicate the involvement of man. The terms, when referring to nucleic acid molecules or polypeptides indicate that they are at least substantially free from at least one other component with which they are naturally associated in nature and as found in nature. In addition, the terms can indicate that the nucleic acid molecules or polypeptides have a sequence not found in nature.

The term “increasing or increase” as used herein, means to induce or increase or activate a level, activity or function or expression of at least one or more transcription factor in the source cell. Preferably, the activity is induced or increased or activated by 5%, 10%, 20%, 30%, 40%, 50%, 60%, 70%, 80%, 90% or more compared to a comparator value (i.e. a control). For example, a control may a source cell into which no expression cassettes encoding the nucleic acid have been introduced. In some embodiments, the amount of transcription factor is increased in a cell (e.g. via an expression cassette directing expression of a nucleic acid molecule encoding one or more transcription factors) relative to a control (e.g. a source cell without said expression cassette(s)). In a further embodiment, “increasing or increase” as used herein means to increase the amount of at least one or more transcription factor proteins in the source cell. In a still further embodiment, increasing the expression comprises “overexpressing” the transcription factor, i.e. increasing the expression of the transcription factor above the endogenous expression level of the transcription factor in the cell.

“Sequence identity” refers to the similarity between amino acid (or nucleotide) sequences. Sequence identity is frequently measured in terms of percentage identity (or similarity or homology); the higher the percentage, the more similar the two sequences are. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et ai, Nucleic acids Research, 12, 387 (1984), BLASTP, BLASTN, and FASTA (Atschul et ai, J. Molec. Biol. (1990) 215, 403). Preferably, a sequence has at least 70% identity, using the default parameters of the BLAST computer program (Atschul et al., J. Mol. Biol. (1990) 215, 403-410) provided by HGMP (Human Genome Mapping Project), to a sequence disclosed herein. The term a “variant” in referring to protein and nucleic acid sequences (including the MEF2C, MEF2D, RXRG and CRX sequences provided herein) are typically characterised by possession of at least 70%, 80%, 85%, 90%, 95%, 96%, 97%, 98%, or 99% sequence identity counted over the full length alignment with the amino acid or nucleotide sequences. The present invention contemplates the use of variants of the transcription factors described herein. The variant could be a fragment of a full-length sequence, a codon-optimised sequence, or a naturally occurring splice variant.

The variant could be a polypeptide or nucleic acid molecule at least 70%, 80%, 85%, 90%, 95%, 98%, or 99% identical to a full length sequence, wherein the fragment is at least 50%, 60%, 70%, 80%, 85%, 90%, 95%, 98%, or 99% as long as the full length wild type polypeptide or nucleic acid molecule, or a domain thereof has a functional activity of interest such as the ability to promote conversion of a source cell type to a target cell type. In some aspects, the domain is at least 100, 200, 300, or 400 amino acids in length, beginning at any amino acid position in the sequence and extending toward the C-terminus. Variations known in the art to eliminate or substantially reduce the activity of the protein are preferably avoided. In some aspects, the variant lacks an N-and/or C-terminal portion of the full-length polypeptide, e.g., up to 10, 20, or 50 amino acids from either terminus is lacking. In some aspects the polypeptide has the sequence of a mature (full length) polypeptide, by which is meant a polypeptide that has had one or more portions such as a signal peptide removed during normal intracellular proteolytic processing (e.g., during co-translational or post-translational processing). In some aspects wherein the protein is produced other than by purifying it from cells that naturally express it, the protein is a chimeric polypeptide, by which is meant that it contains portions from two or more different species. In some aspects wherein a protein is produced other than by purifying it from cells that naturally express it, the protein is a derivative, by which is meant that the protein comprises additional sequences not related to the protein so long as those sequences do not substantially reduce the biological activity of the protein. One of skill in the art will be aware of, or will readily be able to ascertain, whether a particular polypeptide variant, fragment, or derivative is functional using assays known in the art. Other convenient assays include measuring the ability to activate transcription of a reporter construct containing a transcription factor binding site operably linked to a nucleic acid sequence encoding a detectable marker. For example, the skilled person knows how to perform a luciferase assay to determine whether a variant of a transcription factor is a functional variant. For instance, if the variant activates the luciferase assay and/or has at least 50% of the activity of the full-length wild type polypeptide or nucleic acid molecule, it may be defined as a functional variant. Alternatively, the skilled person may compare transcriptomic changes following exposure to a transcription factor and a variant of the transcription factor, for instance using RNAseq, in order to determine whether the variant of the transcription factor is a functional variant. The transcriptomic changes may be upregulation and/or downregulation of gene expression. For instance, if the variant upregulates at least 50% of the genes upregulated by the full-length wild type polypeptide or nucleic acid molecule, it may be defined as a functional variant. Or, if the variant downregulates at least 50% of the genes downregulated by the full-length wild type polypeptide or nucleic acid molecule, it may be defined as a functional variant. Or, if the variant upregulates at least 50% of the genes upregulated by the full-length wild type polypeptide or nucleic acid molecule and downregulates at least 50% of the genes downregulated by the full-length wild type polypeptide or nucleic acid molecule, it may be defined as a functional variant. Alternatively, the skilled person may compare the effects of a transcription factor and a variant of the transcription factor on cell conversion, for instance by comparing the effects on a reduction in one or more characteristics of a source cell (such as Müller glia cells) and/or an increase in one or more characteristics of a target cell (such as cone photoreceptor cells and/or RPE cells), in order to determine whether the variant of the transcription factor is a functional variant. For instance, if at least 50% of the proportion of cells downregulating one or more source cell characteristic (such as the presence of at least one marker and/or at least one morphological trait of a Müller glia cell) following exposure to the full-length wild type polypeptide or nucleic acid molecule also downregulate the one or more source cell characteristic following exposure to the variant transcription factor, the variant may be defined as a functional variant. Or, if at least 50% of the proportion of cells upregulating one or more target cell characteristic (such as the presence of at least one marker and/or at least one morphological trait of a cone photoreceptor cell and/or the presence of at least one marker and/or at least one morphological trait and/or at least one functional characteristic of an RPE cell) following exposure to the full-length wild type polypeptide or nucleic acid molecule also upregulate the one or more target cell characteristic following exposure to the variant transcription factor, the variant may be defined as a functional variant. The skilled person knows how to perform a BDNF expression or secretion assay to determine whether a variant of a transcription factor is a functional variant. For instance, if the variant has at least 50% of the BDNF expression of the full-length wild type polypeptide or nucleic acid molecule, it may be defined as a functional variant. Or, if the variant has at least 50% of the BDNF secretion of the full-length wild type polypeptide or nucleic acid molecule, it may be defined as a functional variant. In certain aspects of the invention a functional variant or fragment has at least 50%, 60%, 70%, 80%, 90%, 95% or more of the activity of the full-length wild type polypeptide or nucleic acid molecule. In certain aspects of the invention a functional variant or fragment has at least 50%, 60%, 70%, 80%, 90%, 95% or more of the activity of a codon optimised molecule disclosed herein; references to the full-length wild type polypeptide or nucleic acid molecule in the above descriptions of how to ascertain whether a particular variant, fragment, or derivative is functional may be replaced by references to a codon optimised molecule disclosed herein. As used herein, the term “functional variant” preferably refers to a molecule, e.g., a polypeptide or nucleic acid molecule that retains at least about 70% or more (including at least 75%, at least 80%, at least 85%, at least 90%, at least 95%, at least 97%, at least 99%, or 100%) of the biological activity of a wild-type molecule or a codon optimised molecule disclosed herein. Biological activity may for instance refer to one or more of activity in a luciferase assay, transcriptomic changes, effects on cell conversion and/or BDNF expression or secretion. Preferably, a “functional variant” is defined by reference to both sequence identity percentage and biological activity. For instance, a “functional variant” may possess at least 70% (including at least 80%, at least 85%, at least 90%, at least 95%, at least 96%, at least 97%, at least 98%, or at least 99% sequence identity counted over the full length alignment with the amino acid or nucleotide sequences) and at least about 70% or more (including at least 75%, at least 80%, at least 85%, at least 90%, at least 95%, at least 97%, at least 99%, or 100%) of the biological activity of a wild-type molecule or a codon optimised molecule disclosed herein. A “functional variant” may possess at least 80% (including at least 85%, at least 90%, at least 95%, at least 96%, at least 97%, at least 98%, or at least 99% sequence identity counted over the full length alignment with the amino acid or nucleotide sequences) and at least about 80% or more (including at least 85%, at least 90%, at least 95%, at least 97%, at least 99%, or 100%) of the biological activity of a wild-type molecule or a codon optimised molecule disclosed herein. A “functional variant” may possess at least 90% (including at least 95%, at least 96%, at least 97%, at least 98%, or at least 99% sequence identity counted over the full length alignment with the amino acid or nucleotide sequences) and at least about 90% or more (including at least 95%, at least 97%, at least 99%, or 100%) of the biological activity of a wild-type molecule or a codon optimised molecule disclosed herein. A “functional variant” may possess at least 95% (including at least 96%, at least 97%, at least 98%, or at least 99% sequence identity counted over the full length alignment with the amino acid or nucleotide sequences) and at least about 95% or more (including at least 97%, at least 99%, or 100%) of the biological activity of a wild-type molecule or a codon optimised molecule disclosed herein.

Variants may comprise one or more amino acid substitutions. Amino acid substitutions are typically conservative substitutions, ie, replacement of one amino acid with another with generally similar properties, such that the overall function is probably not seriously affected. Thus, the amino acids glycine, alanine, valine, leucine and isoleucine can often be substituted for one another (amino acids with aliphatic side chains). Among these potential substitutions, glycine and alanine are used to replace each other (as they have relatively short side chains), valine, leucine and isoleucine replace each other It is preferred that they be used because they have larger aliphatic side chains that are hydrophobic. Other amino acids that can often be substituted for one another are: phenylalanine, tyrosine and tryptophan (amino acids with aromatic side chains); lysine, arginine and histidine (amino acids with basic side chains); aspartic acid and glutamic acid (acidic side) Asparagine and glutamine (amino acids with amide side chains); and cysteine and methionine (amino acids with sulfur containing side chains) are included.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from that of natural cells or is otherwise flanked by a different nucleic acid sequence than that found in nature. An exogenous nucleic acid may also be extra-chromosomal, such as an episomal vector.

The term “expression cassette” is meant to include any type of genetic construct containing a nucleic acid coding for a gene product in which part or all of the nucleic acid encoding sequence is capable of being transcribed and translated, i.e., is under the control of a promoter. An “exogenous expression cassette” is introduced exogenously, originating from outside the organism. As used herein, the term “nucleic acid” encompasses DNA or RNA. “A nucleic acid” as used herein includes but is not limited to a polynucleotide, protein, oligonucleotide, synthetic mRNA molecule, synthetic RNA molecule (for example, repRNA), synthetic DNA molecule, siRNAs, an RNAi, a peptide-nucleic acid (PNA), polypeptide, and peptide.

The nucleic acid can be delivered by a vector such as a viral or a non-viral vector. A viral vector may be one that integrates into the host cell genome or one that does not integrate into the host cell genome such as, but not limited to, lentiviruses, Sendai vectors, Herpes simplex virus (HSV), adenoviruses, adeno-associated viruses (AAV), episomal vectors (e.g. EBV vectors) and retroviruses. In particular, AAVs capsids can be engineered to enhance gene delivery. Capsid variants can be selected to drive protein expression in source cells, for example, AAV6-derived capsid ShH10 has been shown to transduce mainly Müller glia cells in the rat retina, while AAV2.7m8 also has a high level of retinal tropism (Khabou et al., 2016 Biotechnol. Bioeng. 2016;113:2712-2724). Other capsid variants that have been shown to transduce Müller Glia are AAV1, 2, 4, 6, 8, 9, ShH10Y and ShH13 (Koerber, 2009 DOI: 10.1038/mt.2009.184; O'Carroll, 2020 DOI: 10.3389/fnmol.2020.618020). The viral vector may comprise a capsid exhibiting retinal tropism. The viral vector may comprise a capsid for transduction of macroglia, preferably Müller Glia. The viral vector may therefore comprise a capsid selected from the group consisting of AAV1, 2, 4, 6, 8, 9, AAV2.7m8, ShH10, ShH10Y and ShH13. The viral vector may comprise a AAV2 capsid or an AAV2 variant capsid. The viral vector may comprise a AAV6 capsid or an AAV6 variant capsid. AAV capsids can confer specificity of targeting to Müller glia, which can also be achieved/enhanced by using Müller glia specific promoters The vector may or may not be incorporated into the cell's genome. The constructs can include viral sequences for transduction, if desired. Non-viral vectors include but are not limited to liposome, nanoparticle, naked DNA, plasmid, transposon and other means to deliver a nucleic acid molecule into a cell. Another type of vector includes RNA molecules, e.g. mRNA and stabilised RNA, to carry coding genetic information to the cells. Other modalities that are suitable of facilitating the transfer of a nucleic acid into the cell may be used. The term “vector” and “expression vector” are interchangeably used, and they refer to vectors that enable the expression of the nucleic acid encoding the transcription factors. In some aspects the gene of interest is operably linked to another sequence in the vector.

The nucleic acid(s) encoding the transcription factors can be cloned in a monocistronic expression vector, a bicistronic expression vector or a polycistronic expression vector. A monocistronic vector encodes for a single transcription factor, a bicistronic expression vector encodes for two transcription factors and a polycistronic expression vector encodes for more than one transcription factor. Preferably, in a bicistronic or polycistronic expression vector, the nucleic acids encoding the transcription factors are separated by, for example, a P2A, T2A, E2A or F2A linker which induces ribosomal skipping when the protein is being translated so that two or more separate proteins are generated. The nucleic acid encoding the transcription factors are exogenously introduced into the cell.

A “self-replicating RNA” or “self-amplifying RNA” or “repRNA” is an RNA that is capable of continuously replicating, as well as transcribing itself, in a host cell as a replicon without the need for a DNA template.

Accordingly, a repRNA for inducing Müller glia cells to transdifferentiate into cone photoreceptor cells, may comprise a 5′cap and sequences encoding non-structural proteins, at least one or more of MEF2C, MEF2D and RXRG genes, independent ribosome entry sites (IRES), optionally fluorescent marker genes (GFP or mCherry), and 3′poly A tail. The repRNA can further include a CRX gene. The repRNA may comprise a promoter operably linked to a nucleic acid sequence encoding Myocyte Enhancer Factor 2C (MEF2C), or a functional variant thereof, wherein the promoter is for expression of MEF2C in macroglia, and optionally the repRNA may comprise a promoter operably linked to a nucleic acid sequence encoding one or more transcription factor selected from the group consisting of Myocyte Enhancer Factor 2D (MEF2D), Retinoid X Receptor Gamma (RXRG) and Cone-Rod Homeobox (CRX), or functional variants thereof, or any combination thereof, wherein the promoter is for expression of the one or more transcription factor in macroglia. Further, the repRNA molecule can comprise two RNA molecules: a first molecule comprising sequences encoding non-structural proteins required for replication of the RNA, and a second molecule comprising a sequence encoding at least one transcription factor.

A “promoter” refers to a DNA sequence recognized by the synthetic machinery of the cell, or introduced synthetic machinery, required to initiate the specific transcription of a gene. The phrase “under transcriptional control” or “operably linked to” means that the promoter is in the correct location and orientation in relation to the nucleic acid to control RNA polymerase initiation and expression of the gene.

A promoter also optionally includes distal enhancer or repressor elements. A “constitutive promoter” is a promoter that is continuously active and is not subject to regulation be external signals or molecules. For example, constitutive promoters include CMV and CAG. In contrast, the activity of an “inducible promoter” is regulated by an external signal or molecule (for example a transcription factor). A “cell-specific promoter” or “tissue-specific promoter” is a promoter that directs expression of a nucleic acid molecule in particular cells or tissues, for example, cell-specific promoters that are known to lead to expression in Müller glia cells and astrocytes include, retinaldehyde-binding protein 1 (RLBP1) and Glial fibrillary acidic protein (GFAP). Where a promoter preferentially directs expression of a nucleic acid molecule in particular cells or tissues but also directs expression in other cells or tissues at a lower level, it may be referred to as a “cell-selective promoter” or a “tissue-selective promoter”. A number of promoters may be used to induce the expression of the nucleic acid which is operably linked to the promoter sequence. Some other promoters that have been shown to lead to Müller Glia specific expression are ProB2 and GLAST. Promoters suitable for directing expression in Müller Glia include sequences from nine Müller cell-associated genes: CAR2, CD44,GFAP, GLUL, PDGFRA, RLBP1, S100B, SLC1A3, and vimentin (VIM) and variants thereof shown to be able to drive reporter gene expression in Müller Glia by Geller et al (2008) Molecular Vision 2008; 14:691-705, hereby incorporated herein by reference. Promoters described herein by reference to the associated gene include variants of the corresponding promoter as well as “full length” promoters. A “full-length” promoter may comprise around 2000 bases or around 1500 bases upstream relative to the transcriptional start site (TSS). A variant of a promoter may be a truncated promoter consisting of up to around 1000 bases, up to around 900 bases, up to around 800 bases, up to around 700 bases, up to around 600 bases or up to around 500 bases of the “full length” promoter. The bases of the truncated promoter may be consecutive bases proximal to the TSS. “Proximal to” may refer to the furthest base from the TSS being up to 100 bases, up to 90 bases, up to 80 bases, up to 70 bases, up to 60 bases, up to 50 bases, up to 40 bases, up to 30 bases, up to 20 bases, up to 10 bases, up to 5 bases or 0 bases upstream from the TSS. The TSS may be identified using RefSeq sequences (NCBI), for instance those compiled into single data files available at ECRbase. A variant of the promoter may be an evolutionarily conserved region (ECR), preferably the most proximal ECR relative to the TSS. ECR sequences may be identified in full length promoter regions mandating a minimum of 70% sequence homology between human and mouse genomes over a 100 base pair window. The promoter or variant thereof may further comprise the 5′ UTR, or a fragment thereof, associated with the gene. Preferably, the one or more nucleic acids encoding for at least one or more transcription factors is under the control of a GFAP promoter. The use of a GFAP promoter restricts the expression of the transcription factors to Müller glia cells and astrocytes only. Preferably a promoter of between 400 and 800 bp is used, for example, the truncated GFAP promoter gfaABC1D having 681 bp (Lee, 2008 DOI: 10.1002/glia.20622), identified below as SEQ ID NO: 13, which is reported to have essentially the same expression pattern as the 2210 bp gfa2 promoter, and about twofold greater activity.

The GFAP promoter may be a shortened or truncated GFAP promoter. The GFAP promoter may consist of or may comprise SEQ ID NO: 13 (a 681 bp sequence). The GFAP promoter may consist of or may comprise SEQ ID NO: 14 (a 699 bp sequence comprising the sequence of SEQ ID NO: 13).

SEQ ID NO: 13
AACATATCCTGGTGTGGAGTAGGGGACGCTGCTCTGACAGAGGCT
CGGGGGCCTGAGCTGGCTCTGTGAGCTGGGGAGGAGGCAGACAGC
CAGGCCTTGTCTGCAAGCAGACCTGGCAGCATTGGGCTGGCCGCC
CCCCAGGGCCTCCTCTTCATGCCCAGTGAATGACTCACCTTGGCA
CAGACACAATGTTCGGGGTGGGCACAGTGCCTGCTTCCCGCCGCA
CCCCAGCCCCCCTCAAATGCCTTCCGAGAAGCCCATTGAGCAGGG
GGCTTGCATTGCACCCCAGCCTGACAGCCTGGCATCTTGGGATAA
AAGCAGCACAGCCCCCTAGGGGCTGCCCTTGCTGTGTGGCGCCAC
CGGCGGTGGAGAACAAGGCTCTATTCAGCCTGTGCCCAGGAAAGG
GGATCAGGGGATGCCCAGGCATGGACAGTGGGTGGCAGGGGGGGA
GAGGAGGGCTGTCTGCTTCCCAGAAGTCCAAGGACACAAATGGGT
GAGGGGAGAGCTCTCCCCATAGCTGGGCTGCGGCCCAACCCCACC
CCCTCAGGCTATGCCAGGGGGTGTTGCCAGGGGCACCCGGGCATC
GCCAGTCTAGCCCACTCCTTCATAAAGCCCTCGCATCCCAGGAGC
GAGCAGAGCCAGAGCAGGTTGGAGAGGAGACGCATCACCTCCGCT
GCTCGC
SEQ ID NO: 14
ctagtgatctaacatatcctggtgtggagtaggggacgctgctct
gacagaggctcgggggcctgagctggctctgtgagctggggagga
ggcagacagccaggccttgtctgcaagcagacctggcagcattgg
gctggccgccccccagggcctcctcttcatgcccagtgaatgact
caccttggcacagacacaatgttcggggtgggcacagtgcctgct
tcccgccgcaccccagcccccctcaaatgccttccgagaagccca
ttgagcagggggcttgcattgcaccccagcctgacagcctggcat
cttgggataaaagcagcacagccccctaggggctgcccttgctgt
gtggcgccaccggcggtggagaacaaggctctattcagcctgtgc
ccaggaaaggggatcaggggatgcccaggcatggacagtgggtgg
cagggggggagaggagggctgtctgcttcccagaagtccaaggac
acaaatgggtgaggggagagctctccccatagctgggctgcggcc
caaccccaccccctcaggctatgccagggggtgttgccaggggca
cccgggcatcgccagtctagcccactccttcataaagccctcgca
tcccaggagcgagcagagccagagcaggttggagaggagacgcat
cacctccgctgctcgcggggatcc

As used herein, a promoter “for” expression of a gene in macroglia refers to a promoter suitable for expression of the gene in macroglia. To be suitable for expression of a gene in macroglia, the promoter may be a macroglia specific promoter or a macroglia selective promoter. Alternatively, to be suitable for expression of a gene in macroglia, the promoter may be an inducible promoter a constitutive promoter. A promoter which is specific for a cell type other than macroglia, in particular a promoter which is specific for a retinal cell type other than retinal macroglia, is not suitable for expression of a gene in macroglia. The promoter may not be a promoter which is specific for RPE cells (such as a VMD2 promoter or a REG-RPE promoter). The promoter may not be a promoter which is specific for neuronal cells (such as human synapsin1 (hSyn1), neuron-specific enolase (NSE) or calcium/calmodulin dependent protein kinase IIA (CaMKlla)). In some embodiments, the promoter is suitable for selective expression of a gene in macroglia. A promoter which is selective for a cell type other than macroglia, in particular a promoter which is selective for a retinal cell type other than retinal macroglia, is not suitable for selective expression of a gene in macroglia. The promoter may therefore drive a greater level (such as at least a 1.5, 2, 3, 4, 5, 6, 7, 8, 9, 10, 15, 20, 30 or 50 fold increase) of expression of the gene in retinal macroglia relative to the level of expression in another retinal cell type, such as RPE cells or a neuronal cell type, such as cones, rods or RGCs.

“Regulatory element” includes promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods in Enzymology 185, Academic Press, San Diego, Calif (1990), which is hereby incorporated by reference in its entirety. Regulatory elements include those that direct constitutive expression of a nucleotide sequence in many types of source cells and those that direct expression of the nucleotide sequence only in certain source cells (e.g., cell-specific regulatory sequences). Regulatory elements may also direct expression in a temporal-dependent manner, which may or may not also be tissue or cell-type specific.

The protein expression, or amount, of a transcription factor as described herein may be increased by contacting the cell with an agent which activates or increases the expression of the transcription factor. In any embodiment, the agent is selected from the group consisting of: a nucleotide sequence, a protein, an aptamer and small molecule, ribosome, RNAi agent and peptide-nucleic acid (PNA) and analogues or variants thereof. In certain aspects, the agent is exogenous. In some aspects, the agent is a small molecule.

In some aspects, the nucleotide sequence is included as part of a transcriptional activation system (e.g., a gRNA for use in gene editing systems or a TALEN) for increasing the expression of one or more transcription factors. In some aspects, activation of the selected transcription factor(s) may be undertaken in vivo using a transcriptional activation system (e.g., CRISPRa) for increasing the expression of one or more transcription factors.

The present invention has been described using human genes, but other sources may be used as appropriate for the species. Codon optimization can also be performed to optimize expression, and expression vectors, mRNA or repRNA can also be optimized for use. Source cells can be targeted by the use of specific expression vectors and/or the selection of cell-specific promotors.

The present disclosure provides a method of converting, by direct cell conversion or transdifferentiation, a source cell, such as a Müller glia cell or astrocyte, to a cone photoreceptor cell for use in the treatment of retinal degeneration and retinal diseases, including age-related macular degeneration, inherited retinal dystrophies such as retinitis pigmentosa, retinal injury and retinopathies associated with cone photoreceptor loss. Further, the present disclosure provides nucleic acid molecules and compositions for use in the treatment of retinal degeneration or diseases.

Any method as described herein may have one or more, or all, steps performed in vitro, ex vivo or in vivo.

Where the steps of the method as described herein are performed in vitro, the cell or cell population may be delivered in a pharmaceutically acceptable ophthalmic formulation by intraocular injection. When administering the formulation by intravitreal injection, for example, the solution may be concentrated so that minimized volumes may be delivered. Concentrations for injections may be at any amount that is effective and non-toxic, depending on the factors described herein.

The methods described herein can be utilised for autologous therapy (i.e. source cells are isolated from a subject, undergo transdifferentiation to generate cone photoreceptor cells and then injected back into the same subject) or allogeneic therapy (i.e. source cells are isolated from a subject, undergo transdifferentiation to generate cone photoreceptor cells and then injected back into a different subject).

Alternatively, the methods described herein of source cells to cone photoreceptor cells are an in vivo cell conversion method, whereby the nucleic acid molecule(s) encoding at least one or more transcription factors is delivered to subjects and the conversion or transdifferentiation of the source cell to a desired differentiated target cell occurs in vivo. Suitable methods for nucleic acid delivery for transformation of a cell for use with the present invention are believed to include virtually any method by which a nucleic acid (e.g., DNA or RNA) can be introduced into a cell as described herein or as would be known to one of ordinary skill in the art, (e.g., Stadtfeld and Hochedlinger, Nature Methods 6 (5): 329-330 (2009); Yusa et al., Nat. Methods 6:363-369 (2009); Woltjen, et al., Nature 458, 766-770 (9 Apr. 2009)). Such methods include, but are not limited to, direct delivery of DNA such as by ex vivo transfection (Wilson et al., Science, 244:1344-1346, 1989, Nabel and Baltimore, Nature 326:711-713, 1987), optionally with a lipid-based transfection reagent such as Fugene6 (Roche) or Lipofectamine (Invitrogen), by injection (U.S. Pat. Nos. 5,981,274; 5,945,100; 5,780,448; 5,736,524; 5,702,932; 5,656,610; 5,589,466 and 5,580,859; each incorporated herein by reference), including microinjection (Harland and Weintraub, J. Cell Biol., 101:1094-1099, 1985; U.S. Pat. No. 5,789,215, incorporated herein by reference); by electroporation (U.S. Pat. No. 5,384,253, incorporated herein by reference; Tur-Kaspa et al., Mol. Cell Biol., 6:716-718, 1986; Potter et al., Proc. Nat'l Acad. Sci. USA, 81:7161-7165, 1984); by calcium phosphate precipitation (Graham and Van

Der Eb, Virology, 52:456-467, 1973; Chen and Okayama, Mol. Cell Biol., 7 (8): 2745-2752, 1987; Rippe et al., Mol. Cell Biol., 10:689-695, 1990); by using DEAE-dextran followed by polyethylene glycol (Gopal, Mol. Cell Biol., 5:1188-1190, 1985); by direct sonic loading (Fechheimer et al., Proc. Nat'l Acad. Sci. USA, 84:8463-8467, 1987); by liposome mediated transfection (Nicolau and Sene, Biochim. Biophys. Acta, 721:185-190, 1982; Fraley et al., Proc. Nat'l Acad. Sci. USA, 76:3348-3352, 1979; Nicolau et al., Methods Enzymol., 149:157-176, 1987; Wong et al., Gene, 10:87-94, 1980; Kaneda et al., Science, 243:375-378, 1989; Kato et al., J Biol. Chem., 266:3361-3364, 1991) and receptor-mediated transfection (Wu and Wu, Biochemistry, 27:887-892, 1988; Wu and Wu, J. Biol. Chem., 262:4429-4432, 1987); and any combination of such methods, each of which is incorporated herein by reference. This invention is also directed to nucleic acids molecules, vectors, compositions and products which can be used with the disclosed methods. In one example, the composition comprises one or more nucleic acids encoding the transcription factors, or variants thereof, and a pharmaceutically acceptable carrier. Nucleic acids molecules, vectors, compositions and products according to the present invention may be introduced into the source cell by one or multiple expression vectors, for example, lentiviral, adenoviral, or AAV transduction. Nucleic acids molecules, vectors, compositions and products according to the present invention may be administered by intravitreal, suprachoroidal or subretinal injection. The expression vector may be administered by intravitreal, suprachoroidal or subretinal injection.

In a further example, the composition comprises transcription factors, or variants thereof, and a pharmaceutically acceptable carrier, delivered as protein therapeutics. Several methods have been developed to efficiently deliver proteins across the plasma membrane of photoreceptor or retinal pigment epithelium (RPE) cells in vivo, for example, using nucleolin, present on the surface of photoreceptor cell bodies, as a gateway for the delivery of proteins into retinal cells following intravitreal injection.

Pharmaceutically acceptable carriers suitable for the delivery of compositions of the present invention and methods for their preparation will be readily apparent to those skilled in the art. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives and pH buffering agents and the like, for example sodium acetate or sorbitan monolaurate.

“Therapy”, “treatment” and “treating” include both preventative and curative treatment of a condition, disease or disorder. It also includes slowing, interrupting, controlling or stopping the progression of a condition, disease or disorder. It also includes preventing, curing, slowing, interrupting, controlling or stopping the symptoms of a condition, disease or disorder. A “therapeutically effective amount” may vary depending upon one or more of: the subject and disease conditions being treated, the weight and age of the subject, the severity of the disease condition, the manner of administration and the like, which can be determined by one of ordinary skill in the art.

The term “xeno-free (XF)” or “animal component-free (ACF)” or “animal free”, when used in relation to a medium, an extracellular matrix, or a culture condition, refers to a medium, an extracellular matrix, or a culture condition which is essentially free from heterogeneous animal-derived components. For culturing human cells, any proteins of a non-human animal, such as mouse, would be xeno components. In certain aspects, the xeno-free matrix may be essentially free of any non-human animal-derived components, therefore excluding mouse feeder cells or Matrigel™. Matrigel™ is a solubilised basement membrane preparation extracted from the Engelbreth-Holm-Swarm (EHS) mouse sarcoma, a tumour rich in extracellular matrix proteins to include laminin (a major component), collagen IV, heparan sulfate proteoglycans, and entactin/nidogen.

The term “defined”, when used in relation to a medium, an extracellular matrix, or a culture condition, refers to a medium, an extracellular matrix, or a culture condition in which the nature and amounts of approximately all the components are known. A “chemically defined medium” refers to a medium in which the chemical nature of approximately all the ingredients and their amounts are known. These media are also called synthetic media.

A culture, matrix or medium are “essentially free” of certain reagents, such as signalling inhibitors, animal components or feeder cells, when the culture, matrix or medium respectively have a level of these reagents lower than a detectable level using conventional detection methods known to a person of ordinary skill in the art or these agents have not been extrinsically added to the culture, matrix or medium.

Any medium, culture or matrix for any of the steps or sub-steps or throughout the whole process may be xeno-free or defined (as described herein). A medium may be chemically defined. One or more medium or culture for use according to the invention may be free or essentially free of any matrix components, or may include a defined or xeno-free extracellular matrix. For human cells, “xeno-free” means that a medium or culture is free or essentially free of any non-human animal components. Preferably the ex vivo methods of the invention are in accordance with GMP and use xeno-free and/or defined culture and media.

The indefinite articles “a” and “an,” as used herein in the specification and in the claims, unless clearly indicated to the contrary, should be understood to mean “at least one.” Any ranges cited herein are inclusive.

As used herein in the specification and in the claims, the phrase “at least one” or “at least one or more” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Alternative statements of the invention are set out in the following numbered embodiments:
1. A method of converting a source cell to a cone photoreceptor cell by introducing at least one or more transcription factors, or variants thereof, selected from the group consisting of Myocyte Enhancer Factor 2C (MEF2C), Myocyte Enhancer Factor 2D (MEF2D), Retinoid X Receptor Gamma (RXRG) and Cone-Rod Homeobox (CRX), into the source cell, thereby converting the source cell into a cone photoreceptor cell.
2. The method according to embodiment 1, wherein the at least one or more transcription factors, or variants thereof, are encoded on one or more nucleic acids.
3. The method according to embodiment 1, wherein the at least one or more transcription factors, or variants thereof, are introduced into the source cell as transcription factor proteins.
4. The method according to any one of embodiments 1 to 3, wherein the at least one or more transcription factors includes at least two or at least three or at least four of MEF2C, MEF2D, RXRG and CRX, or variants thereof, or any combination thereof.
5. The method according to any one of the preceding embodiments, wherein the at least one or more transcription factors are MEF2C, MEF2D, RXRG and CRX.
6. The method according to any one of the preceding embodiments, wherein the source cell is transdifferentiated to a cone photoreceptor cell.
7. The method according to any one of the preceding embodiments, wherein the source cell is a Müller glia cell or astrocyte.
8. The method according to any one of the preceding embodiments, wherein the source cell is a population of source cells.
9. The method according to any one of the preceding embodiments, wherein the source cell or the population of source cells is derived from a mammal, preferably human.
10. The method according to any one of the preceding embodiments, wherein the at least one or more transcription factors is introduced into the source cell by lentiviral, adenoviral, or AAV transduction.
11. The method according to any one of the preceding embodiments, wherein the source cell is converted to a cone photoreceptor cell when there is an up-regulation of any one or more cell markers of a cone photoreceptor, or a down-regulation of one or more cell markers of a source cell and/or one or more changes in cell morphology from the source to a cone photoreceptor, or any combination thereof.
12. The method according to embodiment 11, wherein the cell marker of a cone photoreceptor is selected from: Cone arrestin (ARR3), GNAT2, and one of S/M/L opsins.
13. The method according to any one of the preceding embodiments, wherein at least 0.01% at least 0.05%, at least 0.10%, at least 0.5% or at least 1.0% of the source cells are converted to photoreceptor cells.
14. The method according to any one of the preceding embodiments, which comprises culturing under suitable conditions for at least 4 days.
15. A cone photoreceptor cell produced by the method of any one of embodiments 1 to 14.
16. A genetically modified cone photoreceptor cell produced according to any one of embodiments 1 to 14, wherein the genetically modified cone photoreceptor cell is genetically modified with one or more exogenous nucleic acids encoding at least one or more transcription factors selected from the group comprising MEF2C, MEF2D, RXRG and CRX, or variants thereof.
17. A nucleic acid molecule for converting a source cell to a cone photoreceptor cell comprising a nucleic acid sequence encoding at least one or more transcription factors selected from the group consisting of MEF2C, MEF2D, RXRG and CRX, or variants thereof.
18. An exogenous expression cassette comprising one or more nucleic acid molecules according to embodiment 17, wherein the one or more nucleic acid molecules is operably linked to a promoter, preferably a cell-specific promoter.
19. A cone photoreceptor cell according to embodiments 15 or 16, a nucleic acid molecule according to embodiment 17, or an exogenous expression cassette according to embodiment 18, for use in the treatment of retinal disease or degeneration.
20. A composition for converting a source cell to a cone photoreceptor cell comprising at least one or more transcription factor proteins selected from the group consisting of MEF2C, MEF2D, RXRG and CRX or variants thereof, the cone photoreceptor cell according to embodiments 15 or 16, the nucleic acid molecule according to embodiment 17 or the exogenous expression cassette according to embodiment 18, and a pharmaceutically acceptable carrier.
21. The composition of embodiment 20 for use in the treatment of retinal disease or degeneration.
22. A method of treating retinal disease or degeneration in a subject comprising administering to a retina of the subject in need thereof a therapeutically effective amount of at least one or more transcription factor proteins or a therapeutically effective amount of one or more nucleic acid molecules encoding the at least one or more transcription factors, selected from the group consisting of MEF2C, MEF2D, RXRG and CRX, or variants thereof, wherein the increase of at least one factor selected from the group consisting of
MEF2D, MEF2C, RXRG and CRX, or variants thereof, induces the conversion of a source cell to a cone photoreceptor cell, and wherein the source cell is a Müller glia cell or astrocyte.
23. A method of treating retinal disease or degeneration in a subject comprising administering to a retina of the subject in need thereof a therapeutically effective amount of the cell or a population of cells according to embodiments 15 or 16.
24. A method of treating retinal disease or degeneration in a subject comprising administering to a retina of the subject in need thereof a therapeutically effective amount of the composition of embodiment 20.
25. The method according to any one of the preceding embodiments, wherein the retinal disease or
26. The method according to any one of the preceding embodiments, wherein the method increases the number of cone photoreceptor cells in the retina.

In an alternative definition, according to a first embodiment the present invention provides a method of converting a source cell to a cone photoreceptor cell by introducing at least one or more transcription factors, or variants thereof, selected from the group consisting of Myocyte Enhancer Factor 2C (MEF2C), Myocyte Enhancer Factor 2D (MEF2D), Retinoid X Receptor Gamma (RXRG) and Cone-Rod Homeobox (CRX), into the source cell, thereby converting the source cell into a cone photoreceptor cell.

In an alternative definition, according to a second embodiment of the invention, there is provided a method of converting a source cell to a cone photoreceptor cell by introducing at least one or more transcription factors, or variants thereof, selected from the group consisting of Myocyte Enhancer Factor 2C (MEF2C), Myocyte Enhancer Factor 2D (MEF2D) and Retinoid X Receptor Gamma (RXRG), into the source cell; and wherein the at least one or more transcription factors optionally further includes Cone-Rod Homeobox (CRX), or a variant thereof.

According to one aspect, the at least one or more transcription factors, or variants thereof, are encoded on one or more nucleic acids.

According to an alternative aspect, the at least one or more transcription factors, or variants thereof, are introduced into the source cell as transcription factor proteins.

In an alternative definition, in some embodiments, at least one or more transcription factors includes at least two or at least three or at least four of MEF2C, MEF2D, RXRG and CRX, or variants thereof, or any combination thereof. In further embodiments, the at least one or more transcription factors includes at least two or at least three of MEF2C, MEF2D and RXRG, or variants thereof, or any combination thereof. In a still further embodiment, the at least one or more transcription factors are MEF2C, MEF2D, RXRG and CRX.

In any aspect the source cell may be a Müller glia cell and/or astrocyte. The source cell may be a single cell or a population of source cells. The source cell or the population of source cells may be derived from a mammal, preferably human. The source cell may be transdifferentiated to a cone photoreceptor cell according to any method of the invention.

In a preferred embodiment, the at least one or more transcription factors is introduced into the source cell by lentiviral, adenoviral, or AAV transduction.

In any method of the invention described herein, the source cell is converted to a cone photoreceptor cell when there is an up-regulation of any one or more cell markers of a cone photoreceptor, or a down-regulation of one or more cell markers of a source cell and/or one or more changes in cell morphology from the source to a cone photoreceptor, or any combination thereof. Preferably, the cell marker of a cone photoreceptor is selected from: Cone arrestin (ARR3), GNAT2, and one of S/M/L opsins.

In an alternative definition, the present invention also provides a cone photoreceptor cell produced by a method according to the invention or a genetically modified cone photoreceptor cell produced by a method according to the invention, wherein the genetically modified cone photoreceptor cell is genetically modified with one or more exogenous nucleic acids encoding at least one or more transcription factors selected from the group comprising MEF2C, MEF2D, RXRG and CRX, or variants thereof. Alternatively, the at least one or more transcription factors, or variants thereof, is selected from the group consisting of MEF2C, MEF2D and RXRG, and the one or more nucleic acids optionally further encodes CRX, or a variant thereof.

In an alternative definition, the present invention further provides a nucleic acid molecule for converting a source cell to a cone photoreceptor cell comprising a nucleic acid sequence encoding at least one or more transcription factors selected from the group consisting of MEF2C, MEF2D, RXRG and CRX, or variants thereof. Alternatively, the at least one or more transcription factors, or variants thereof, is selected from the group consisting of MEF2C, MEF2D and RXRG, and optionally further CRX, or a variant thereof.

The present invention still further provides an exogenous expression cassette comprising one or more nucleic acid molecules according to the invention, wherein the one or more nucleic acid molecules is operably linked to a promoter, preferably a cell-specific promoter.

In any method described herein, the method may further include the step of administering the cells or cell population including a cell, a nucleic acid molecule or an exogenous expression cassette, produced according to a method of the invention, to an individual.

The present invention provides methods for use in the treatment of retinal disease or degeneration including the treatment of age-related macular degeneration (AMD), retinitis pigmentosa (RP) or diabetic retinopathy.

In an alternative definition, the present invention also provides compositions for converting a source cell to a cone photoreceptor cell comprising at least one or more transcription factor proteins selected from the group consisting of MEF2C, MEF2D, RXRG and CRX or variants thereof and a pharmaceutically acceptable carrier. Further embodiments provide compositions for converting a source cell to a cone photoreceptor cell comprising a cone photoreceptor cell or population of cone photoreceptor cells produced according to a method of the present invention; a nucleic acid molecule produced according to a method of the present invention; or an exogenous expression cassette produced according to a method of the present invention; and a pharmaceutically acceptable carrier. The compositions may be for use in the treatment of retinal disease or degeneration.

In an alternative definition, in any aspect of a method of the invention described herein, the source cell is a Müller glia cell, and the transcription factors, or variants thereof, are:
(a) MEF2C, MEF2D, RXRG and CRX;
(b) MEF2C, MEF2D and RXRG;
(c) MEF2C, MEF2D and CRX;
(d) MEF2D, RXRG and CRX;
(e) MEF2C, RXRG and CRX;
(f) MEF2C and MEF2D;
(g) MEF2D and RXRG;
(h) MEF2D and CRX;
(i) MEF2C and RXRG;
(j) MEF2C and CRX;
(k) RXRG and CRX;
(l) MEF2D;
(m) MEF2C;
(n) RXRG; or
(o) CRX.

The increased expression of transcription factors used herein was accomplished through cloning of transcription factor genes into lentiviral and/or AAV expression vectors. However, this was exemplary only and any expression vector could be used. Alternatively, mRNA or self-replicating/self-amplifying RNA (srRNA, saRNA or repRNA) could be used, or even intact functional proteins.

DNA, RNA, and protein can be introduced into cells in a variety of ways, including e.g., viral transduction, microinjection, electroporation, and lipid-mediated transfection. Ocular drug delivery is known to be challenging due to the presence of effective epithelial barriers protecting eye tissue. Viruses, such as lentivirus or AAVs, are often injected into the vitreous or locally to the neural retina by sub-retinal injection to bypass the epithelial barriers. RNA can also be delivered to retinal cells using cell penetrating peptides (CCP), synthetic and natural short amino acid sequences able to cross cellular membrane thanks to a transduction domain, for example, TAT-fusion, for example using the HIV-1-TAT protein. TAT has also been used for protein delivery. Other cell-penetrating peptides (CPP) are also known, and intact proteins can be delivered using CPPs as fusion proteins, as well as by noncovalent CPP/protein complexes.

At the current time, AAVs are preferred for gene therapies as they are non-immunogenic, for example, LUXTURNA® (voretigene neparvovec-rzyl) received market authorisation from the FDA in 2017 as an in vivo gene therapy to treat one subtype of retinitis pigmentosa, involving an AAV2 expressing RPE65. However, there is much research into non-viral delivery methods, such as plasmid DNA, lipid/nucleic acid lipoplexes, cationic liposomes, cationic polymers, nanoparticles, and cationic metal complexes. Using genome engineering techniques, for example CRISPR/Cas9, it is possible to selectively activate the required proteins, rather than by cell delivery of DNA, RNA or protein. CRISPR activation (CRISPRa) is an optimized method for specific gene overexpression that uses an inactivated CRISPR-Cas9 system (dCas9) to upregulate target genes within their native context.

The foregoing description has been presented only for the purposes of illustration and is not intended to limit the disclosure to the precise form disclosed. The details of one or more aspects of the disclosure are set forth in the accompanying description above. Although any methods and materials similar or equivalent to those described herein can be used in the practice or testing of the present disclosure, the preferred methods and materials are now described.

### EXAMPLES

### Example 1—Identification of Transcription Factors Involved in the Transdifferentiation of Müller Glia Cells to Photoreceptors

Publicly available single-cell RNA sequencing data of adult human retina and retinal organoids (Cowan et al., Cell 2020) was added to the applicant's proprietary platform (see, e.g., WO2017106932). The platform predicted and ranked transcription factors that are involved in the transdifferentiation of macroglia, including Muller glia cells and astrocytes, to cone photoreceptors. Based on the coverage percentage of the network and other criteria, the top four ranked transcription factors were identified as MEF2C, MEF2D, RXRG and CRX. These transcription factors were taken forward for in vitro, in vivo and ex vivo experiments.

### Example 2—Lentiviral generation to encode MEF2C, MEF2D, RXRG and CRX

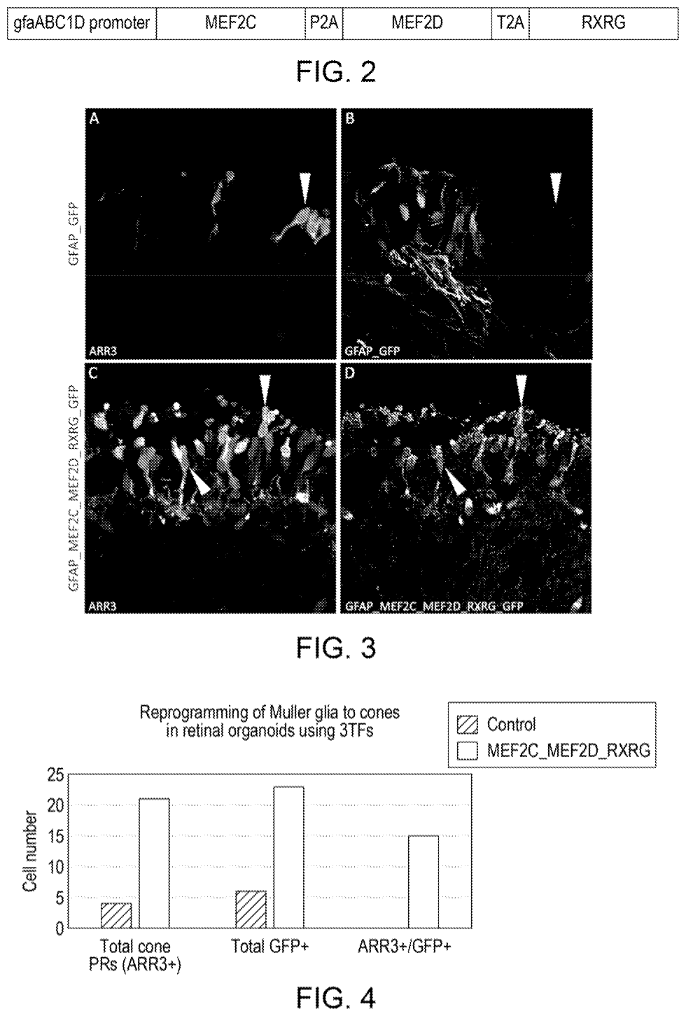

Lentiviral vectors were generated by cloning MEF2C, MEF2D, RXRG and CRX individually. Polycistronic vectors were also generated encoding the combination of MEF2C, MEF2D, and RXRG, (FIG. 2) and the combination of MEF2C, MEF2D, RXRG and CRX. The transcription factors were under the control of a GFAP promoter and expressed GFP. The following expression cassettes were generated (with GFAPp standing for GFAP promoter):
GFAPp-MEF2C-GFP
GFAPp-MEF2D-GFP
GFAPp-RXRG-GFP
GFAPp-CRX-GFP
GFAPp-MEF2D-CRX-mCherry

### Example 3—Transduction of Retinal Organoid Differentiation with the Transcription Factors

In order to assess whether MEF2C, MEF2D, RXRG and CRX alone or in a combination could transdifferentiate Müller glia cells to cone photoreceptors, human retinal organoids were used as in vitro models.

Human iPSCs were derived from fibroblasts using the Yamanaka factors and a stable iPSC line was generated (38F) maintained in mTeSR media. Retinal organoids were differentiated from iPSCs according to the protocol in Cowan et al., Cell 2020. iPSC colonies were dissociated to generate embryoid bodies using EDTA (0.5 mM in PBS) at 37C. Embryoid bodies were grown in suspension in 2 mL of mTeSR media supplemented with 10 uM blebbistatin in petri dishes. Embryoid bodies were cultured for 125 days.

On day 125, retinal organoids were moved to a low-attachment 96 well plate in 100 μl media. Lentiviral mixes were made up to 100 ul with media (RDM (N2) 30 FBS+Taurine+0.5 μM RA +1uM 9-cis RA +6ug/ml polybrene). The media was removed from organoids and replaced by the viral mix and returned to the incubator. Retinal organoids were transfected with either GFAPp-GFP (control virus), GFAPp-MEF2C-GFP, GFAPp-MEF2D-GFP, GFAPp-RXRG-GFP, GFAPp-CRX-GFP, GFAPp-MEF2C-MEF2D-RXRG-GFP, GFAPp-MEF2C-RXRG or GFAPp-MEF2D-CRX-mCherry.

On day 1 post-transfection, media was topped up to 200 μl with (RDM (N2) +FBS +Taurine+ +0.5 μM RA +1 uM 9-cis RA) and then at the end of the day, organoids were transferred to a 25 well low attachment plate in 1ml media. After day 2 post-transfection, media was changed twice a week for up to 4 weeks.

### Example 4—Co-Localisation of the Transcription Factors and Cone Arrestin Detected by Immunochemistry

Retinal organoids at day 14 post-transfection were washed and cryopreserved before sectioning onto slides. Immunofluorescence staining was performed on 10 μm thick cryo-sections by using a primary antibody against ARR3, a cone specific marker. The slides were then incubated with a secondary antibody which contains a fluorophore to visualise the cells expressing ARR3.

Fluorescence imaging of the slides were undertaken to analyse the expression of GFP and ARR3 under a Leica epi-fluorescence microscope and a Zeiss LSM 910 confocal microscope.

Successful reprogramming events were determined by the co-localisation of GFP, driven by the GFAP promoter and linked to transcription factor expression, with a cone photoreceptor-specific marker (ARR3), which was not observed in controls. In organoids that had received GFAP_TF_GFP vectors however, clear co-localisation between GFP and ARR3 was observed, indicative of a conversion taking place (GFP+/ARR3+).

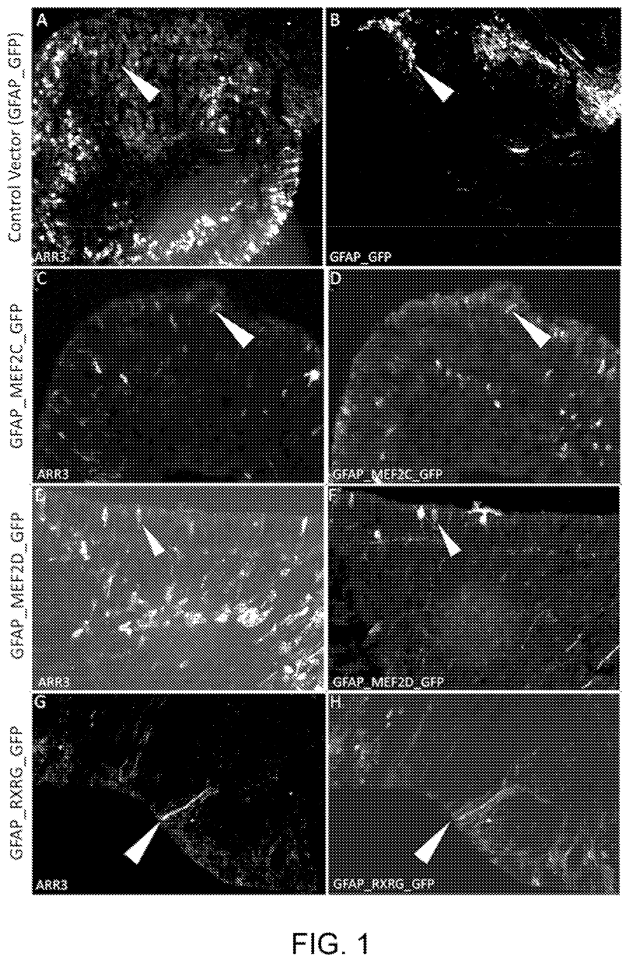

No expression of GFP was observed in cone photoreceptors following transduction of human retinal organoids with control lentivirus expressing GFP, see FIG. 1 (A and B). Co-expression of GFP and ARR3 was observed following transduction of human retinal organoids with lentiviral vectors encoding MEF2C (FIG. 1 (C and D)), MEF2D (FIG. 1 (E and F)), RXRG (FIG. 1 (G and H)) alone and the combination of MEF2C-MEF2D-RXRG (FIG. 3). Importantly, the morphology of converted cells was consistent with photoreceptors in the outer nuclear layer, where rudimentary outer segments were also observed. No evidence of co-expression of GFP and ARR3 was observed following transduction of human retinal organoids with the corresponding lentiviral vector encoding CRX.

Quantification of total cone receptors (ARR3+), total GFP+ cells and reprogrammed cones (ARR3+/GFP+) in human retinal organoids after transduction with three transcription factors is shown in FIG. 4. It should be noted that the human retinal organoids, as used, also have pre-existing cones (ARR3+) and there exists a certain amount of inherent variability between retinal organoids.

### Example 5—Transduction of Mouse Retinal Explants with the Transcription Factors

To further assess transduction of the transcription factors in vivo, reprogramming of Müller glia to cone photoreceptors was carried out in post-mortem mouse retinal explants.

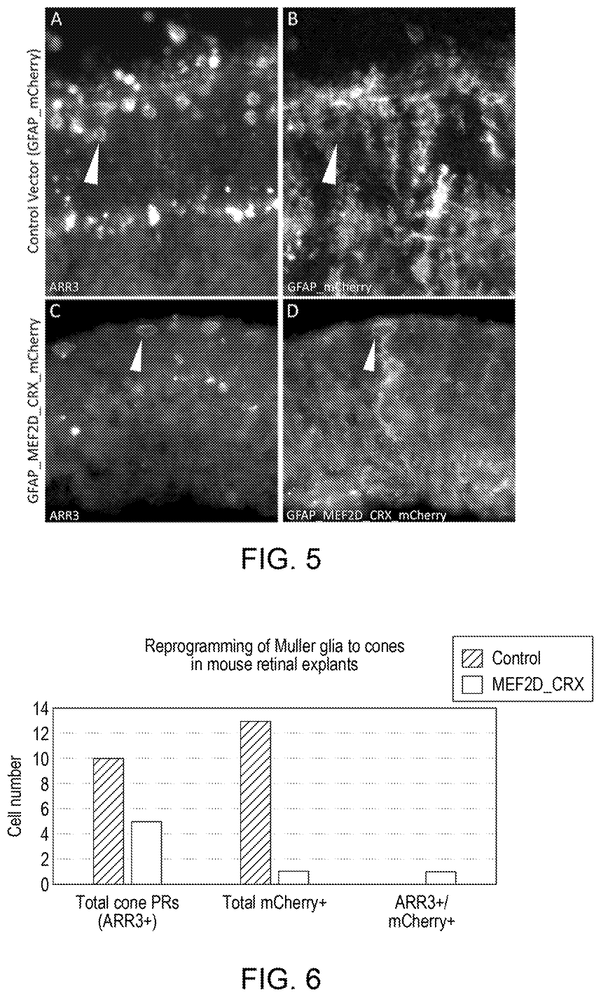

FIG. 5 (A and B) shows that when a control AAV was used that drives mCherry expression under the GFAP promoter, no successful reprogramming events of Müller glia to cone photoreceptors were observed (white arrowheads illustrate the region that mCherry marked photoreceptors would be expected to be seen). Alternatively, when an AAV2-variant was used that encodes two MOGRIFY v.2.5 system predicted transcription factors (MEF2D_CRX) (C and D), co-localisation of mCherry with ARR3 was observed, illustrating that the transcription factors drive the conversion of Müller glia cells to cones (the arrows illustrate mCherry coloured photoreceptors and the morphology of the photoreceptors is clear to see).

FIG. 6 Quantification of total cone photoreceptors (ARR3+), total GFP+ cells, and reprogrammed cones (ARR3+/GFP+) in mouse retinal explants after transduction with AAVs encoding MOGRIFY v.2.5 system predicted factors. No successful reprogramming of Muller glia to cones was observed when the control vector was used, however, when bicistronic AAV2-variant vectors encoding two transcription factors (MEF2C_RXRG and MEF2D_CRX) were used, successful reprogramming of Müller glia to cones (ARR3+/mCherry+) was observed.

### Example 6—Photosensitivity and Electrical Output of Ex Vivo Human Retinal Explants

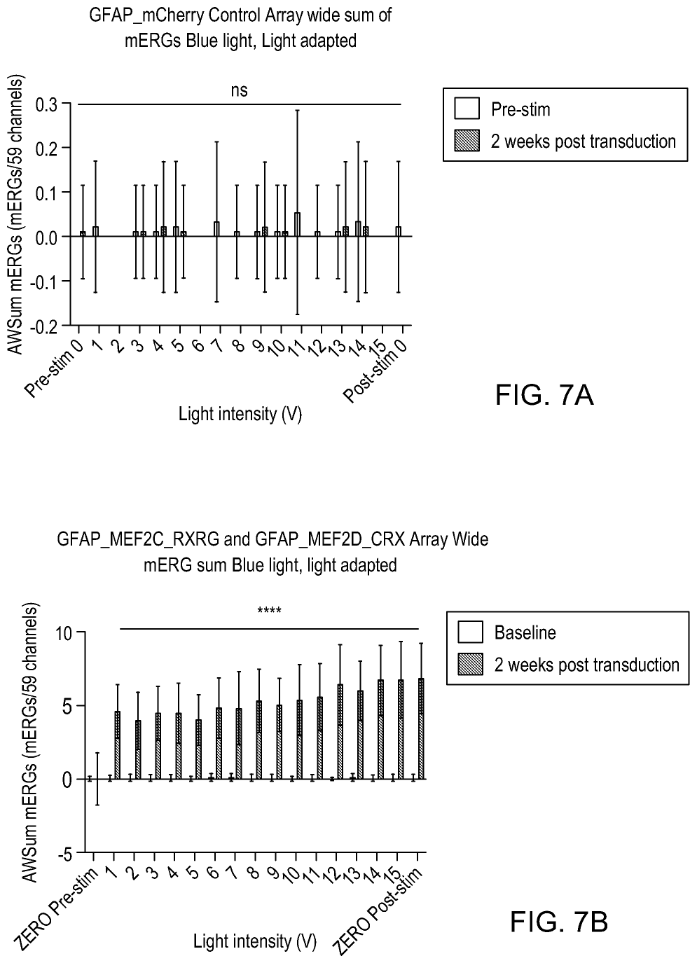

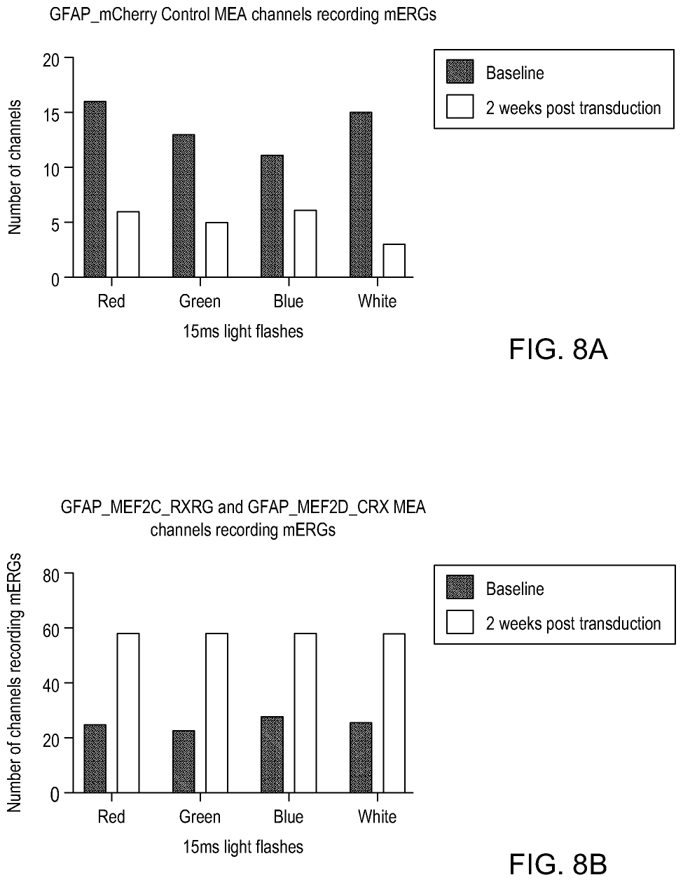

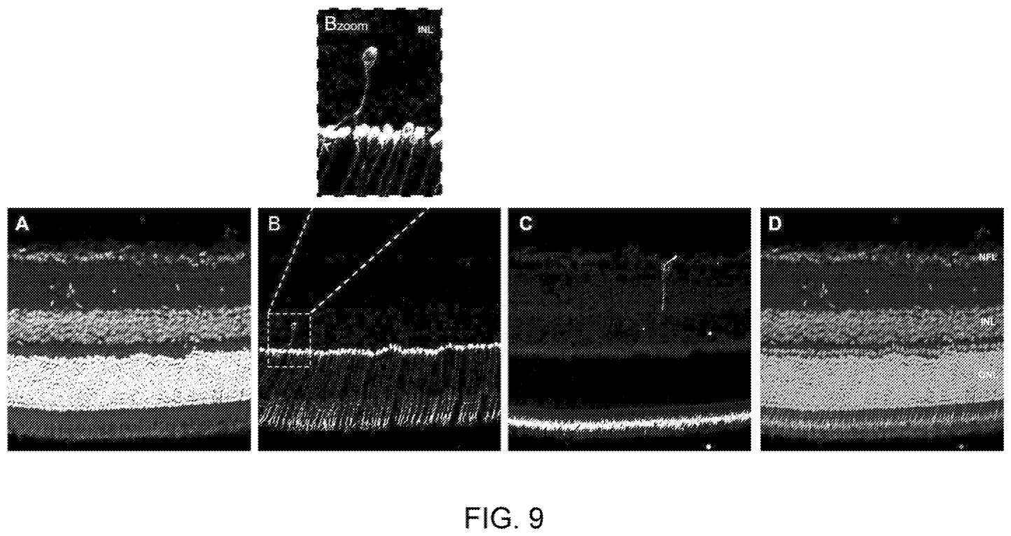

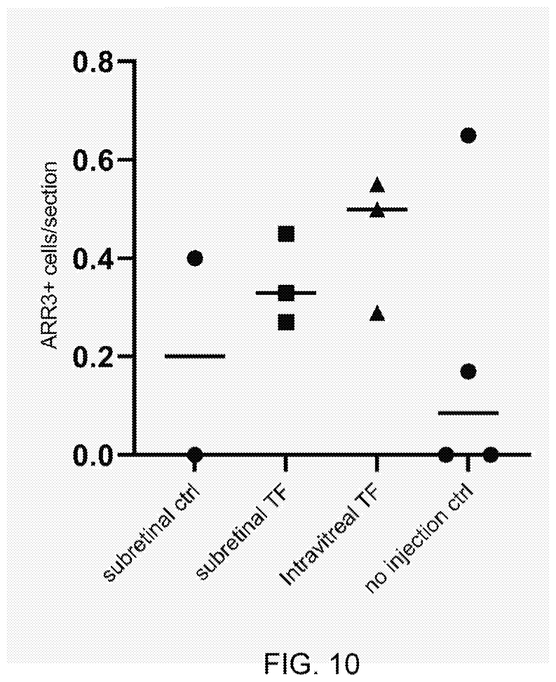

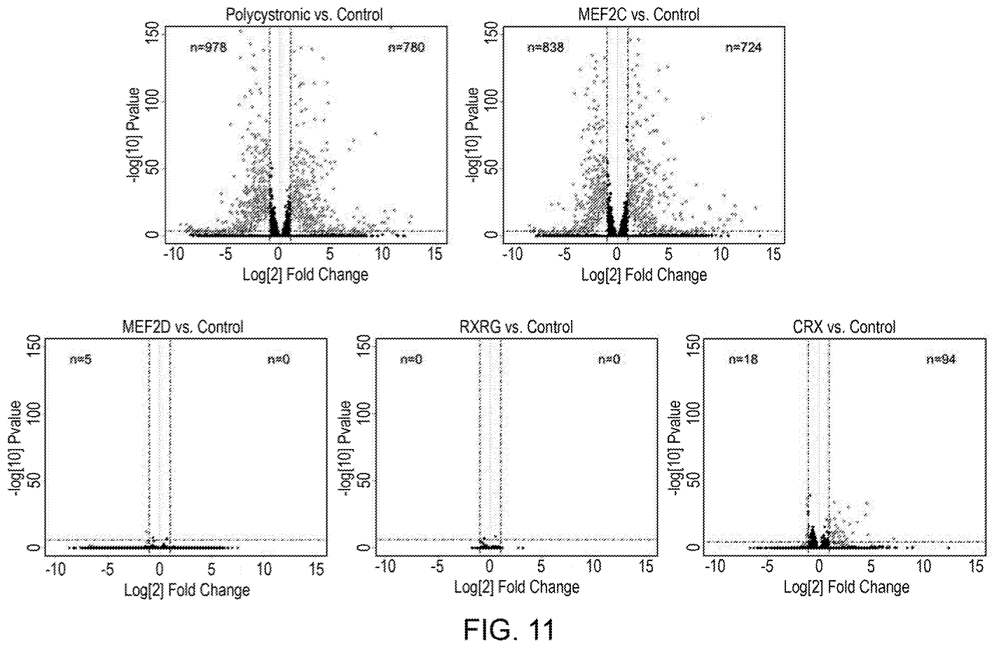

The AAVs encoding the transcription factors were also evaluated in cadaveric human retinal explants by measuring their photosensitivity and electrical output before and after viral transduction. An increase in photosensitivity in human retinal explants after treatment suggests a high potential of therapeutic value in FIGS. 7A, 7B, 8A and 8B.

Explants were placed ganglion cell layer down onto the central recording electrode grid of the multielectrode arrays (60MEA200/30iR-TI, Multi Channel Systems, GmbH: TiN electrodes, SiN insulating layer, TIN contact pads and tracks. Electrodes 30 μm diameter, 200 um spacing, ‘square packing’ with an internal reference electrode). Once explant was centred on the array grid, ‘harps’ (ring of pure platinum, supporting nylon mesh) were placed over the explant to prevent floating when media was added. 800 ul complete Neurobasal-A (47ml Neurobasal A, 1 ml of 50x B27 supplement, 500ul of 100x N2 supplement, 500 ul of 100 mM Sodium Pyruvate, 500 ul Glutamax, 50 ul of 50 nM Taurine, 60.8ul of 1 mM N-Acetyl-L-cysteine and 500 ul of 100x Pen/Strep) with 1 um 9 cis-retinal was added to the MEA well. Arrays were placed in tissue culture dish in 37° C. incubator (5% CO2). Media was changed every 2 days. Electrophysiological recordings are performed prior to transduction, normally the day after dissection, and 14 days post transduction. MEAs were placed into a twin-MEA ‘clamshell’ amplifier (MEA2100-2x60-System). Once located in the recording apparatus, the explants on the arrays were further sealed off from the immediate surroundings with the aid of mini-bioincubators (Ndimension (Science and Engineering) Ltd.) that have been 3D-printed using biocompatible polymers. Two types of mini-bioincubator are used, opaque/matt black for ‘dark adapted’ recordings, and transparent/'water clear' for ‘light-adapted recordings’. These devices were equipped with a port for the delivery of sterile, 5% CO2-in-air (110 cm3min-1) in order to maintain the pH of the bicarbonate-buffered culture media. A solid state sensor module (ExplorIR-WH-20 Gas Sensing Solutions) was incorporated to obtain data concerning the temperature, relative humidity and CO2 concentration (in either % or ppm) in the atmosphere above and surrounding the MEAs at a sampling rate of 2 Hz. The temperature of the fluid within the MEA chambers was held at 37° C. (+0.1° C.) by TC02 temperature controller units (Multi Channel Systems) and the heating plates incorporated into the MEA2100-2x60amplifier. Wide-band neural signals (1 Hz-3.3 KHz) were obtained from the MEAs using a data acquisition system centred around a freely programmable Digital Signal Processor Interface (MCS-IFB; ADC Resolution: 16 bit; USB 3.0 link: Multi Channel Systems). Data was acquired and stored to disk using the MCRack package (Version: 4.6.2. Multi Channel Systems) at a sampling rate of 25 KHz per channel. Two sets of 2nd order IIR Butterworth filters were instituted in the MCRack virtual instrument, for display during both the recording, and the subsequent ‘off-line’ analysis. The first, with a bandpass of 300 Hz-3.3 KHz isolates the spiking activity of retinal ganglion cells (RGCs), and the second with a bandpass of 1-20 Hz reveals mERG waveforms. An f =50 Hz ‘bandstop’ or ‘notch’ filter is also employed (Q =2.0) in order to remove any ‘mains hum’. Thus, for ‘off-line’ analysis, two ‘datastream objects’ are produced, one for the spiking activity of RGCs, and one for mERG slow waves. Samples were left to adjust and dark adapt for an hour before recordings begin. Light exposure was achieved using a purpose-built, LED-based ‘photic stimulator’, with the light being transmitted to illuminate the underside of the explants via twin fibre optic light pipes—the stimulation is ‘full-field’ (i.e. the entire sample). The intensity, pulse-width and frequency of the light flashes is controlled by external electronics, and TTL pulses, marking the time of the stimuli was sent from a special interface box to the data acquisition system. Light exposure is always performed in the same order: Red light 15 ms, flashes, Green light 15 ms flashes, Blue light 15 ms flashes, White light 15 ms flashes, White light is flashes. Flashes were spaced 30s apart. Responses to 3 flashes at each of 15increasing light-intensity steps is recorded at each intensity (for later ‘averaging’ purposes). These intensity steps are designed to cover the entire range of both ‘dark-adapted’ and ‘light-adapted’ vision. 5 minute no light intervals were used between colours to allow the explant to ‘recover’, especially from any ‘photobleaching’ effects. Once all recordings were completed under dark adaptation, the room lights are switched on to light adapt the explants and the opaque/matt black mini-bioincubator was replaced by the transparent/'water clear' equivalent to allow ambient light in. This light adaptation period is 15-30 minutes. All colour and white recordings are repeated. The RGC spiking activity datastream object was analysed using the NeuroExplorer package (V. 5.129. 64-bit version Nex Technologies). In order to render the unit activity compatible with import into the NeuroExplorer package, which is designed to consider the firing of action potentials as ‘point processes’, it is necessary to extract and represent ‘detected spikes’ as ‘timestamps’. This is achieved by directing the filtered spike data stream through a ‘Spike Sorter’ virtual instrument in MCRack. Within this instrument, a detection voltage threshold was set with a polarity to match the dominant polarity expressed by the spike waveforms; this threshold is set to 4 standard deviation units of the RMS noise levels as estimated by the software. A second element of the Spike Sorter instrument produces ‘spike cut-outs’ of the detected spike waveforms, typically with digitised signal 1 ms pre-trigger (threshold crossing), 2 ms post-trigger, and with a ‘dead time’ of 2 ms, within which a second spike cannot registered. The Default Timestamp Frequency of 20,000 Hz in NeuroExplorer is changed to match the MCRack digitizing frequency of 25 KHz per channel. The spike timestamps and logs of the TTL pulses from the photic stimulator control interface are processed within NeuroExplorer to produce both continuous running, and peri-stimulus time histograms of this activity for all electrodes in parallel (1 s bins), and this information is then ported to Excel for subsequent graphing and comparative analysis. Data generated from any individual electrode channel displaying correlates of degenerative processes that ‘swamp’ the recordings are logged at this time. The mERG datastream object was analysed in two stages. Firstly, the frequency of ‘thresholded’ mERGs was determined in a manner synonymous with the previous detection of RGC spikes, but in this case, the data is ported directly to Excel via a text-file intermediate for subsequent graphing and analysis. In contrast, information concerning the relative amplitudes, and times of occurrence of mERG ‘a’ and ‘b’ wave components was determined using a combination of ‘peak/trough’ detection and ‘active cursors’ in MCRack and Spike 2 (Cambridge Electronic Design V8.07x64). The changing mERG profiles of both ‘control’ and ‘treated’ retinal explants under the different light stimulation conditions (light/dark adapted, colour, intensity), and relative to the time of transduction is examined, and the relative ‘a’-wave and ‘b’-wave fingerprints determined via scatterplots and cluster analysis.

FIGS. 7A and 7B show human retinal explants tested for light responsiveness using Multi Electrode Arrays (MEA) pre-transduction and 2 weeks post-transduction with either AAV_GFAP_mcherry (control, A) or with AAV_GFAP_MEF2C_RXRG and AAV_GFAP_MEF2D_CRX (TF treated, B). In FIG. 7A, the mERG

Awsum decreased over the 2 week period of culture post transduction in comparison to baseline (P=0.088, NS, paired t-test). In FIG. 7B, the mERG Awsum significantly increased over the 2 week period post transduction with the TFs (p<0.0001, paired t-test). These findings suggest that there has been an increase in light responsive cells (i.e. photoreceptors) over the 2 week period following transduction with TFs but not the mCherry control. The recordings were performed under light adaptation conditions using coloured light, thus excluding rod photoreceptor light responses and further selecting for cone photoreceptor responses.

FIG. 8 quantifies the number of MEA channels registering mERGs. There were 60 channels on an MEA chip, however 1 is the reference electrode, therefore the maximum number of channels that can be recorded is 59. In FIG. 8A, where the explant was treated with the GFAP_mCherry control AAV, the number of channels recording mERG responses decreased over the 2 week culture period post transduction, whereas in FIG. 8B, where the explant was treated with the GFAP_ME2C_RXRG and GFAP_MEF2D_CRX AAVs, there is an increase in the number of channels recording mERGs. These findings show that the number of light responses has increased across the MEA, and indicates mERGs are being detected 2 weeks post treatment in channels that did not record mERGs at baseline. This is consistent with the generation of novel cones because of cell conversion due to the delivery of MEF2C, MEF2D, RXRG and CRX.

### Example 7—ARR3+ Cells can be observed Outside the Outer Nuclear Layer following In Vivo Delivery of the Transcription Factors

Reprogramming of Muller glia to cone photoreceptors by in vivo delivery of the transcription factors were assessed in C57BL/6JRj male mice to validate the findings of successful Müller glia to cone conversions observed in mouse retinal explants stated in Example 5. All mice were treated at day 0 with a 2x109 vector genome copies per eye intravitreal (IVT) dose of an AAV encoding B-catenin (GFAP_Bcatenin_GFP) for activation and induction of GFAP expression in the Müller glia cells prior to delivery of the TFs.

For intravitreal injections, the anesthetized animals were placed under a stereoscope, and a small incision in the sclera-choroid exposing the vitreous chamber was performed using a 30G needle near the limbus. A microsyringe with a 33G needle attached was used to inject a total volume of 1 ul AAV vector(s) into the intravitreal space.

On day 14, mice received a second injection. At this point the mice receiving a second injection were split into three groups. The first group received an IVT injection of the two bicistronic TF containing AAV2-variant vectors (GFAP_MEF2C-RXRG_myc and GFAP_MEF2D_CRX_mCherry) at a total dose of 2x109 vector genome copies per eye. The second group received a subretinal injection of the two bicistronic TF containing AAV2-variant vectors (GFAP_MEF2C-RXRG_myc and GFAP_MEF2D_CRX_mCherry) at a total dose of 2x109 vector genome copies per eye. The third group received a AAV2-variant control AAV encoding the mCherry fluorophore (GFAP_ mCherry) at a total dose of 2x109 vector genome copies per eye.

For subretinal injections, the anesthetized animals were placed under a stereoscope and a small incision with a 30G needle was performed in the temporal side of the conjunctiva/sclera in order to expose the choroid. The same needle was used to create a small opening in the temporal side of the choroid. The cornea was punctured in order to reduce the intraocular pressure. A Hamilton microsyringe was filled with 1 ul solution and was introduced into the subretinal space through the exposed choroid. The solution was injected into the subretinal space and injection was confirmed using in vivo SD-OCT imaging.

At the end point, day 42, animals were sacrificed, and eyes were enucleated. Eyes were immersion fixed in 4% paraformaldehyde and posterior eye cups cryoprotected in an increased sucrose gradient. Eye cups were subsequently embedded in optimal cutting temperature compound (OCT) and frozen with liquid nitrogen cooled isopentane for histology and immunohistochemistry. Immunofluorescence staining was performed on 12 μm thick cryo-sections using primary antibodies against ARR3, a cone specific marker and mCherry for GFAP_MEF2D_CRX_mCherry visualisation. Slides were then incubated with fluorescent secondary antibodies and investigated for ARR3 and mCherry expression/localisation using a Zeiss LSM 910 confocal microscope. ARR3+ cells outside the photoreceptor layer were quantified by manual counting during microscopic investigation.

Immunohistochemical detection of ARR3 and mCherry in mouse retina sections from these three groups of AAV treated mice plus the ‘no injection wild type control mice’ showed that ARR3+ cells can be identified outside the photoreceptor layer in all four groups. A very low baseline occurrence of ARR3+ cells was observed in a few retinal sections from the AVV control and the ‘no injection wildtype control’ groups. However, we observed a trend in which the number of ARR3+ cells outside the outer nuclear layer (ONL) increased with TF-treatment independent of administration route (subretinal vs. Intravitreal).

The cell body/nucleus of these ARR3+ cells resided in the INL and not the ONL as expected for cone photoreceptors. However, an INL location is consistent with the location of Müller glia nuclei and thus suggesting that these may represent converted Muller glia cells. Combined, this suggests that new cone photoreceptors may be formed following ocular TF delivery. Overall, this is consistent with the Müller glia reprogramming events identified in the transduced retinal organoids and mouse retinal explants.

In contrast to the observations from TF-treated retinal organoids and mouse retinal explants, no co-localisation of mCherry and ARR3 was observed in any of the TF-treated or AAV control groups. This difference may be due to the variation in time points investigated (day 14-post transduction for retinal organoids and mouse retinal explants). The earlier time points may have captured converting ‘Müller glia to cone’ hybrid cells that still have a significant GFAP promoter activity. Whereas the later in vivo ‘4 weeks post TF delivery’ time point may only capture more cone-like cells where the GFAP promoter is no longer active, resulting in a cessation of the mCherry fluorophore expression. The lack of mCherry expression in the ARR3+cells outside of the photoreceptor layer may also be because the conversion is independent of the GFAP_MEF2D_CRX_mCherry AAV and only mediated by the GFAP_MEF2C-RXRG_myc AAV (not detectable by immunohistochemistry).

No mCherry signal was observed in photoreceptors within the photoreceptor layer in any of the groups investigated supporting the Müller glia specific expression of the promoter system used.

### Example 8—Transcription factor contributions analysed in transcriptomic analysis of 2D Müller glia Derivation of Müller glia cells:

At day 125 of retinal organoid differentiation, individual organoids were dissociated to single cell suspensions using Neurosphere Dissociation Kit (Miltenyi Biotec-130-095-943) according to the manufacturers protocol. The Muller glia population was expanding by seeded the retinal organoid single cell suspension on a T25 flask coated with bovine fibronectin at a concentration of 2 ug per cm². Adherent cells were cultured in a medium consisting of DMEM high glucose, 10% foetal bovine serum, 40 ng/ml human EGF and 40ng/ml human bFGF at 37° C. and 5% CO2 (Hence forth referred to as 2D MG medium). Muller glia cells were expanded in 2D MG medium over approximately 2-3 weeks until 90% confluency of a T175 was achieved. To cryopreserve the confluent T175 cultures Müller glia cells were dissociated with TrypLE and cryopreserved in aliquots of 1×10\hat{ }6 cells in DMEM high glucose, 10% foetal bovine serum and 10% dimethyl sulfoxide.

To assess the composition of the retinal organoid-derived culture RT-qPCR for markers for various retinal cell types was performed. Following cryopreservation total RNA was extracted from a vial of frozen cells using the Arcturus PicoPure RNA isolation kit and cDNA was generated from the extracted RNA using the Bioline SensiFAST™ cDNA Synthesis Kit. qPCR was performed on the resulting cDNA to confirm the expression of Müller glia markers CRYM, GLUL, VIM & PAX6 and the absence photoreceptor markers ARR3, GUCA1C and NR2E3.

### Generation of RNA Samples:

Retinal organoid-derived Müller glia cells were seeded into 6 well plates at a density of 10,000 cells per well and maintained in 2D MG medium. 24 hours after seeding cells were transduced in quadruplicate with lentiviral vectors encoding 1 of the 5 following expression cassettes:
GFAP_MEF2C_MEF2D_RXRG_GFP
EF1a_GFP
EF1a_CRX_GFP.
EF1a_RXRG_GFP
EF1a_MEF2D_GFP.
EF1a_MEF2C_GFP

To facilitate transduction, each viral vector was combined with 2ml of 2D MG medium supplemented with 6 ug/ml of polybrene and incubated with the cells at 37° C. and 5% CO2 overnight. Transduction efficiency was above 90%, based on GFP expression. After 24 hours the viral supernatants were aspirated and replaced with fresh 2D MG medium. 7 days after transduction total RNA was extracted from the cells using the Arcturus PicoPure RNA isolation kit according to the manufacturers protocol. RNA-Seq libraries were prepared and sequenced at a depth of 12G per sample on the Illumina NovoSeq 6000 by Novogene, Cambridge. Reads were demultiplexed and aligned to the human genome (GRCh38) via STAR (version 2.7.10b) and transcript counting was performed via RSEM (version 1.3.3). Differential gene expression analysis was performed via DESeq2 (version 1.26.0). Genes were considered significantly differentially expressed between TF-treated conditions and control if they had a p value less than or equal to 0.05 and an absolute log fold change greater than 1.

### Analysis of RNA Samples:

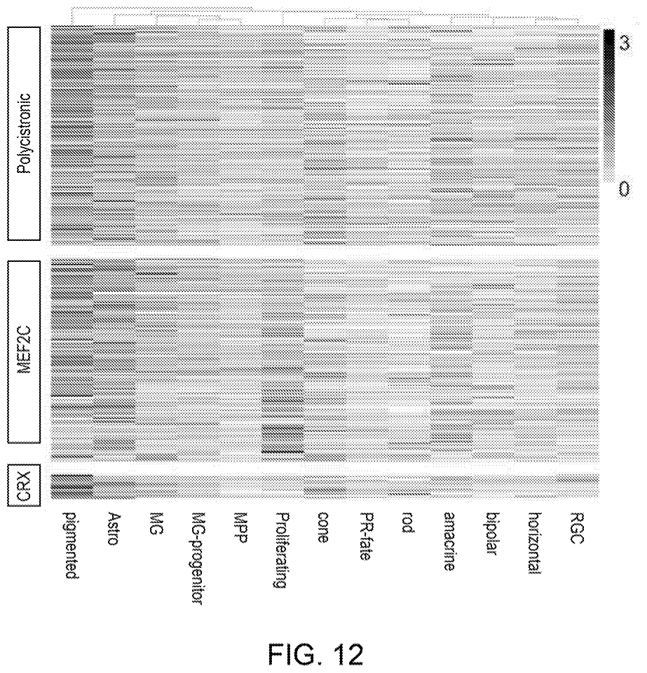

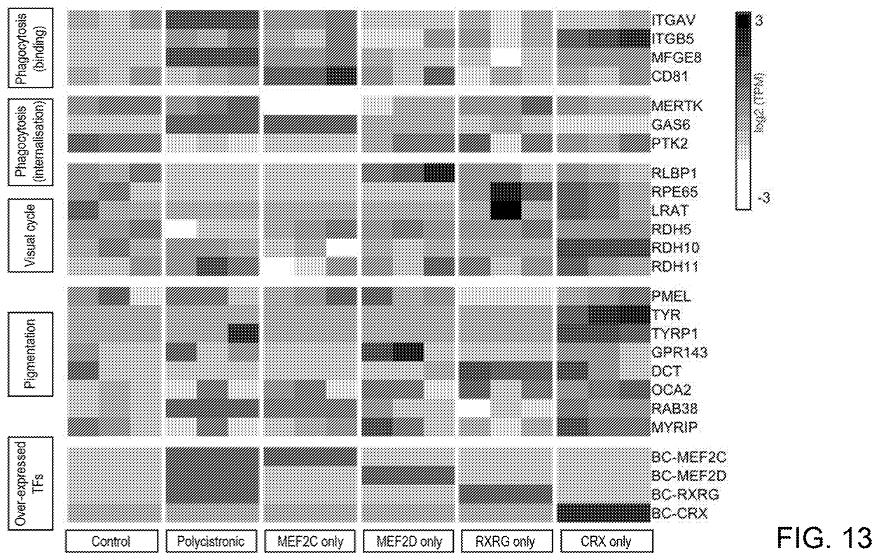

We observed a statistically significant up and downregulation of genes in response to the addition of MEF2C, CRX, or the polycistronic (MEF2C, MEF2D, RXRG), but not when MEF2D or RXRG were added alone (FIG. 12). Surprisingly, MEF2C alone and the polycistronic (MEF2C, MEF2D and RXRG) each caused many more significantly upregulated and downregulated genes than CRX alone. The number of differentially expressed genes increased between MEF2C alone and the polycistronic suggesting a synergistic effect of MEF2C, MEF2D and RXRG.

If we investigate the expression of the statistically significantly upregulated genes in the Cowan et al., reference scRNAseq retinal organoid dataset, we see that the polycistronic, MEF2C alone, and CRX alone upregulated genes are more highly expressed in reference RPE (i.e. pigmented) cells, than any other reference cell type (FIG. 13). In the MEF2C-treated condition, a subset of the upregulated differentially expressed genes are also upregulated in reference proliferating cells. In the polycistronic-treated condition, we also observe a subset of the upregulated differentially expressed genes are also upregulated in reference cone photoreceptors. FIG. 13 also emphasises how MEF2C and the polycistronic (MEF2C, MEF2D and RXRG) each upregulate many more genes in the Cowan et al. reference scRNAseq retinal organoid dataset than CRX.

The effect of the polycistronic, MEF2C alone, and CRX alone on upregulating genes associated with RPE cells raises the question of whether any RPE functions could be activated by these transcription factors. Retinal pigment epithelium (RPE) cells have several well-defined functions, including: phagocytosis of cone photoreceptor outer segments; visual cycle (a biochemical processes that regenerates chromophore); and pigmentation. Genes involved in phagocytosis (binding) by RPE cells include ITGAV, ITGB5, MFGE8 and CD81. Genes involved in phagocytosis (internalisation) by RPE cells include MERTK, GAS6 and PTK2.Genes involved in the visual cycle in RPE cells include RLBP1, RPE65, LRAT, RDH5, RDH10 and RDH11.Genes involved in pigmentation of RPE cells include PMEL, TYR, TYRP1, GPR143, DCT, OCA2, RAB38and MYRIP.

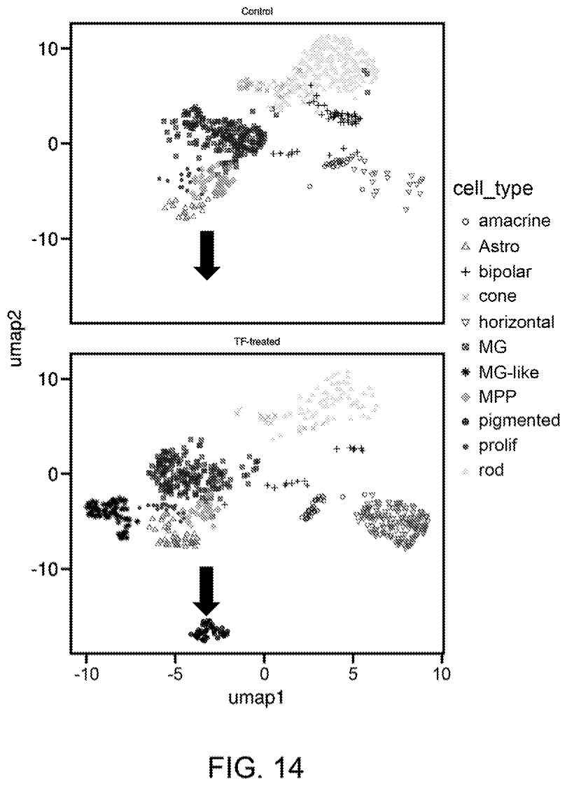

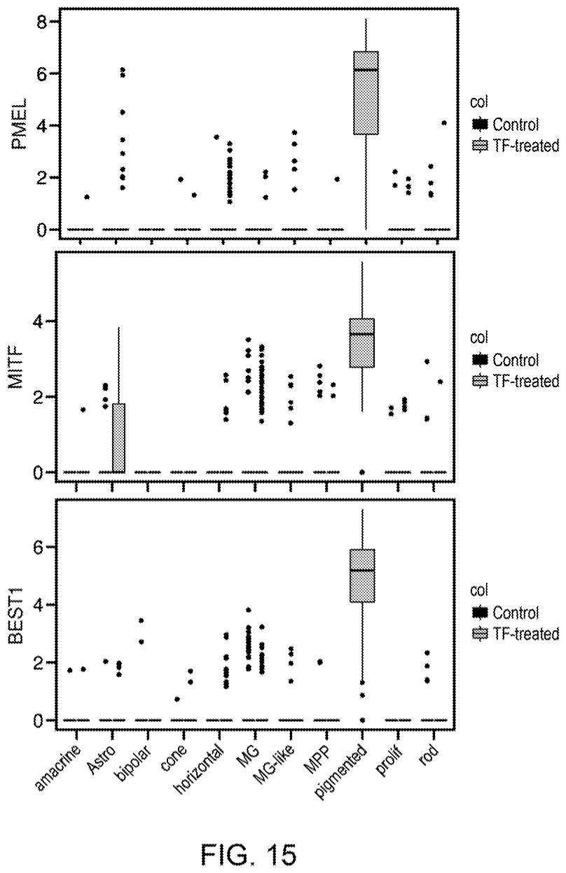

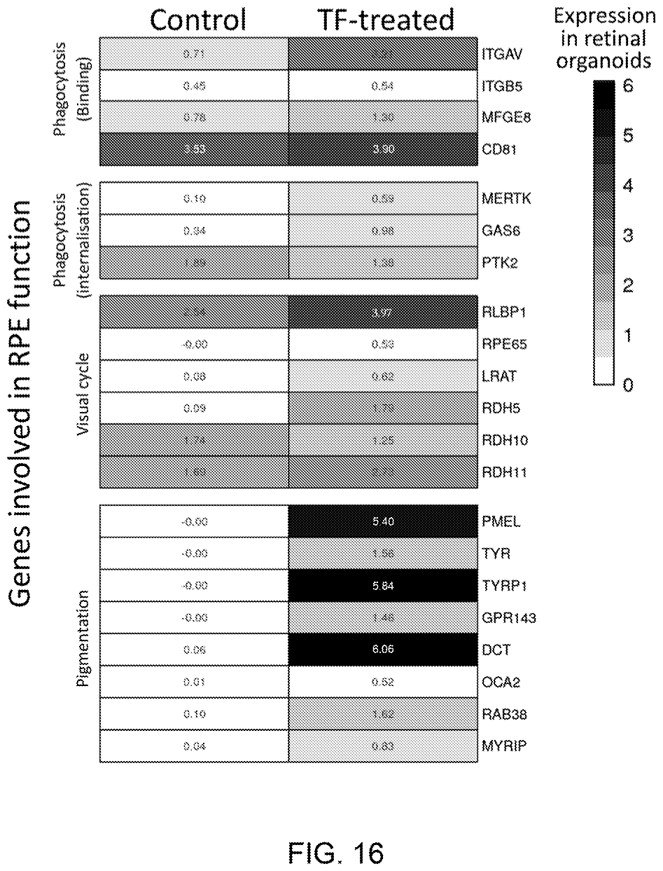

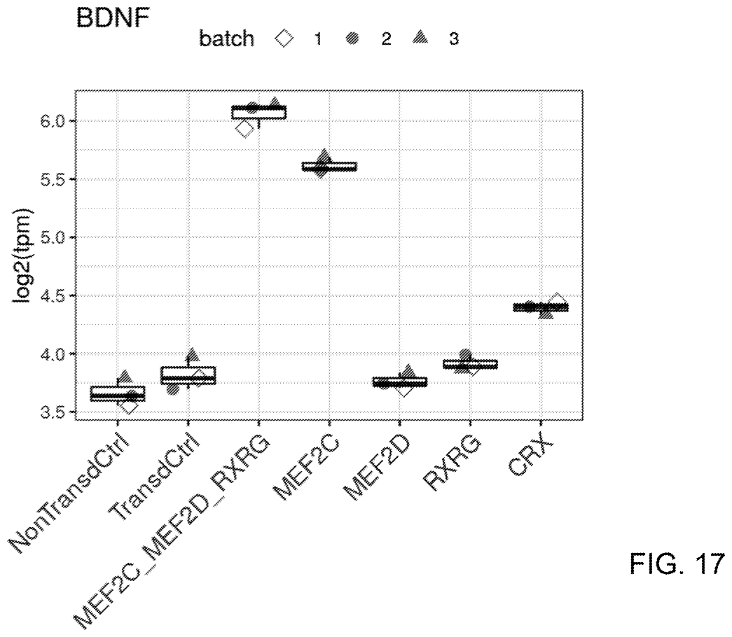

FIG. 14 illustrates the effect of each transcription factor condition on genes involved in RPE function, based on bulk sequencing data from 2D MG cultures. This analysis suggests that genes associated with phagocytosis (binding) may be upregulated by MEF2C and the polycistronic (MEF2C, MEF2D and RXRG) to a greater extent than by CRX alone (which appears to have some effect) and clearly to a greater extent than MEF2D and RXRG. It is possible that CRX alone has the greatest effect on upregulating genes associated with the visual cycle and/or pigmentation in this system, raising the possibility that additive effects of CRX when combined with MEF2C or the polycistronic, could upregulate genes involved in a wide array of RPE functions. However, as these data are generated from bulk sequencing experiments, their interpretation is complicated by the potential for divergent effects on MG. For example, if any transcription factors are directing the cells to transdifferentiate into more than one cell type (for instance via a multipotent, proliferating and/or progenitor cell), genes underlying key RPE functions could be upregulated in some cells adopting an RPE-like phenotype whilst also being downregulated in other cells adopting another cell phenotype (such as a cone-like phenotype). Therefore, while these data suggest that some RPE functions could be differentially activated by MEF2C, the polycistronic and CRX, they require further investigation in a system where RPE-like cells can be isolated and specifically analysed.

### Example 9—Retinal Organoid Transcriptomics

Retinal organoids were generated from the 38F iPSC cell line as described (Cowan et al., 2020). Retinal organoids at week 16 were treated with 1 mm purmorphamine to activate Müller glia. Retinal differentiation media (RDM supplemented with FBS, N2 and Taurine) and fresh purmorphamine was replenished every 2-3 days for a total of two weeks.

Individual organoids were transduced in 96 well plates containing, RDM and 2.5E+06 IVP lentivirus containing 6 mg/ml polybrene and supplemented with 0.5 mm 9-cis-retinoic acid. A total of 16 retinal organoids were transduced with polycistronic v551 lentivirus (encoding MEF2C, MEF2D and RXRG) and 6 control GFP (v770) lentiviruses.

Transduced organoids were maintained for 3 weeks with media changes every 2-3 days. Post transduction organoids were dissociated (Neurosphere dissociation kit, Miltenyi Biotech) and fluorescent positive cells were isolated by FACS sorting (BD FacsAria III).

FACS sorted cells were mixed with spike-in cells, iPSC and k562, and loaded on a 10X Genomics Chromium instrument according to manufacturer's instructions (10X Genomics, chemistry version 3.1). TF-treated and control samples were processed in parallel lanes. Single cell RNA-Seq libraries were prepared according to manufacturer's instructions (10X Genomics, chemistry version 3.1) and sequenced at a depth of at least 50,000 reads per cell on the Illumina NovoSeq 6000 by Novogene, Cambridge. Reads were demultiplexed and aligned to the human genome (GRCh38) and empty barcodes were removed via Cell Ranger (version 6.0.0). Low quality cells were removed and counts were normalised in line with common practices for scRNAseq analysis, see for example Basics of Single-Cell Analysis with Bioconductor, Robert Amezquita et al, Version: 1.6.2 (Chapters 1-3). Batch effect was evaluated using spike-in cells, which demonstrated no batch effect. At this point, spike-in cells were removed from the dataset and normalised counts were re-computed. The remaining data was clustered using shared nearest neighbour-based unsupervised clustering methods. Each cluster was annotated with a cell type based on the expression of well-described retinal cell type markers.

In the TF-treated condition, we observed the presence of an RPE-like population that was absent in the control (GFP-treated) condition (see FIG. 13). As shown in in FIG. 14, his RPE-like group upregulated canonical RPE-markers including Bestrophin 1 (BEST1), melanocyte inducing transcription factor (MITF), and premelanosome protein (PMEL).

Since the RPE-like cells of this Example were identified using single cell sequencing, the expression of genes associated with RPE function can be analysed at single cell resolution in the RPE-like population, without any complicating effects of other cell types that may be represented in the bulk sequencing data of Example 8. The expression of genes underlying the same well-defined functions, including: phagocytosis of cone photoreceptor outer segments; visual cycle (a biochemical processes that regenerates chromophore); and pigmentation were therefore analysed in the RPE-like population of cells produced by

TF treatment of retinal organoids. We observed an upregulation in the expression of genes involved in each of these RPE functions in the transcription factor (TF)-treated RPE-like populations vs controls (FIG. 16). We observed the starkest increase in pigmentation-related genes. These data suggest that the RPE-like cells produced by the polycistronic (MEF2C, MEF2D and RXRG) may exhibit RPE functional characteristics associated with phagocytosis, visual cycle and pigmentation. Furthermore, since the data of Example 8 suggest that a significant proportion of the effect of the polycistronic (MEF2C, MEF2D and RXRG) is exhibited following treatment with MEF2C alone, it is believed that MEF2C alone may be sufficient to produce RPE-like cells exhibiting RPE functional characteristics associated with phagocytosis, visual cycle and pigmentation.

### Example 10—Increased Brain-Derived Neurotrophic Factor Secretion from TF-Treated Retinal Organoid-Derived Müller Glia Cells

Transcriptomic data of TF-treated cells and retinal organoids, described in Examples 8 & 9, shows that the addition of lentivirus-delivered GFAP_MEF2C_MEF2D_RXRG_GFP induces the transdifferentiation of a subset of Müller glia to cells with RPE-like transcriptional profiles.

The ‘golden standard’ for characterising RPE cells is usually based around the fact that they are specialised epithelial cells that polarise and differentiate when situated in a monolayer resulting in a significant transepithelial resistance. Furthermore, evaluation of specialised functions such as melanosome production and presence of RPE markers only found in fully differentiated epithelialized RPE cells such as bestrophin 1 and RPE65 are usually also part of the assessment. However, these classical RPE functions/markers are not all meaningful to pursue in our setting as we are converting individual Müller glia cells into isolated individual RPE-like cells that are not embedded in a monolayer and thus not expected to be capable of polarizing and differentiating to this level within the timelines of the experiments performed.

Another well-established function of the RPE is the secretion of several protein factors including Brain-Derived Neurotrophic Factor (BDNF). Functional evaluation of the Müller glia-derived RPE-like cells through assessment of secretion levels of established secreted factors of the RPE is thus more meaningful.

Increased BDNF expression is seen in retinal organoid-derived Muller glia cells treated with the polycistronic vector or the monocistronic MEF2C vector compared to other individual TFs and the ‘no transduction’ and transduction controls. To evaluate BDNF secretion levels from TF treated Müller glia cells, BDNF levels was quantified in conditioned culture media by enzyme-linked immunoassay (ELISA).

Retinal organoid-derived Müller glia cells derived as in Example 8 and were seeded into 12 well plates at a density of 10,000 cells per cm² and maintained in 2D MG medium (which is described in Example 8). 24 hours after seeding the cells were transduced in triplicate with lentiviral vectors encoding either the GFAP_GFP control or GFAP_MEF2C_MEF2D_RXRG_GFP. To facilitate transduction each viral vector was combined with 1ml of 2D MG medium supplemented with 6 ug/ml of polybrene and incubated with the cells at 37° C. and 5% CO2 overnight. The following day the viral supernatant was aspirated and replaced with 1ml of fresh 2D MG medium. 4 days post transduction the medium was replenished with an equivalent 1 ml volume of 2D MG medium. On day 6 the spent medium was aspirated and replaced with 500 ul of 2D MG medium before harvesting the supernatant on day 9. Supernatants for BDNF secretion quantification were harvested and centrifuged at 2000 g, 4° C. for 10 mins and diluted 1:2 prior to analysis using the Human BDNF SimpleStep ELISA® kit.

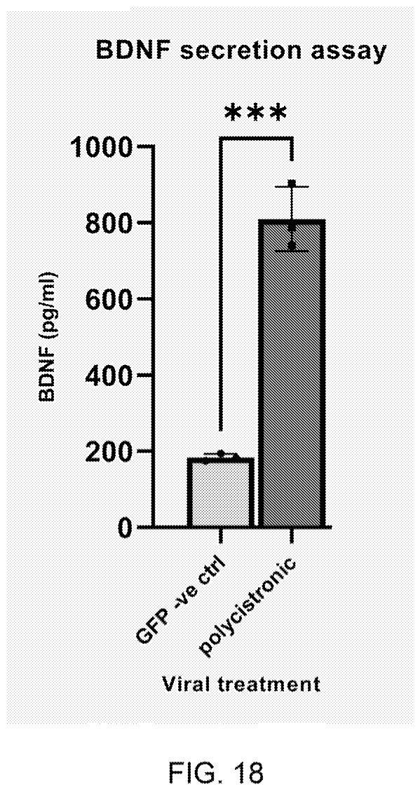

BDNF secretion from Muller glia cells treated with the polycistronic lentivirus vector encoding three TFs (GFAP_MEF2C_MEF2D_RXRG_GFP) increased significantly (4-fold, p=0.0002) compared to cells treated with the GFP only transduction control (GFAP_GFP) (FIG. 18) suggesting that the TF treated Müller glia cells gain RPE functionalities.

1. A nucleic acid molecule comprising a promoter operably linked to a nucleic acid sequence encoding Myocyte Enhancer Factor 2C (MEF2C), or a functional variant thereof, wherein the promoter is for expression of MEF2C in macroglia.

2. The nucleic acid molecule according to claim 1, wherein the functional variant of MEF2C comprises an amino acid sequence at least 90% identical to SEQ ID NO: 1

3. The nucleic acid molecule according to any preceding claim, wherein the nucleotide sequence encoding MEF2C or a functional variant thereof comprises:
(a) SEQ ID NO: 3,
(b) SEQ ID NO: 2, or
@ a nucleotide sequence having at least 60% identity to SEQ ID NO: 3 or SEQ ID NO: 2.

4. The nucleic acid molecule according to any preceding claim, comprising a promoter operably linked to a nucleic acid sequence encoding one or more transcription factor selected from the group consisting of Myocyte Enhancer Factor 2D (MEF2D), Retinoid X Receptor Gamma (RXRG) and Cone-Rod Homeobox (CRX), or functional variants thereof, or any combination thereof, wherein the promoter is for expression of the one or more transcription factor in macroglia.

5. The nucleic acid molecule according to claim 4, wherein the nucleic acid sequence one or more transcription factor selected from the group consisting of Myocyte Enhancer Factor 2D (MEF2D), Retinoid X Receptor Gamma (RXRG) and Cone-Rod Homeobox (CRX), or functional variants thereof, or any combination thereof is operably linked to the same promoter as the a nucleic acid sequence encoding Myocyte Enhancer Factor 2C (MEF2C), or a functional variant thereof.

6. The nucleic acid molecule according to any one of claims 4 to 5, wherein
(a) the functional variant of MEF2D comprises an amino acid sequence at least 90% identical to SEQ ID NO: 4; and/or
(b) the functional variant of RXRG comprises an amino acid sequence at least 90% identical to SEQ ID NO: 7; and/or
(c) the functional variant of CRX comprises an amino acid sequence at least 90% identical to SEQ ID NO: 10.

7. The nucleic acid molecule according to any one of claims 4 to 6, wherein the nucleotide sequence encoding MEF2D or a functional variant thereof comprises:
(a) SEQ ID NO:6,
(b) SEQ ID NO: 5, or
(c) a nucleotide sequence having at least 60% identity to SEQ ID NO: 6 or SEQ ID NO: 5.

8. The nucleic acid molecule according to any one of claims 4 to 7, wherein the nucleotide sequence encoding RXRG or a functional variant thereof comprises:
(a) SEQ ID NO: 9,
b) SEQ ID NO: 8, or
(c) a nucleotide sequence having at least 60% identity to SEQ ID NO: 9 or SEQ ID NO: 8.

9. The nucleic acid molecule according to any one of claims 4 to 8, wherein the nucleotide sequence encoding CRX or a functional variant thereof comprises:
(a) SEQ ID NO: 12,
(b) SEQ ID NO: 11, or
(c) a nucleotide sequence having at least 60% identity to SEQ ID NO: 12 or SEQ ID NO: 11.

10. The nucleic acid molecule according to any one of claims 4 to 9, comprising a nucleotide sequence encoding MEF2C and a nucleotide sequence encoding RXRG.

11. The nucleic acid molecule according to any one of claims 4 to 10, comprising a nucleotide sequence encoding MEF2C, a nucleotide sequence encoding MEF2D and a nucleotide sequence encoding RXRG.

12. The nucleic acid molecule according to any one of claims 1 to 11, wherein the nucleic acid sequence encoding MEF2C is 5′ relative to a nucleic acid sequence encoding one or more further transcription factor.

13. The nucleic acid molecule according to any one of claims 4 to 12, wherein a nucleic acid sequence encoding RXRG is 3′ relative to a nucleic acid sequence encoding MEF2C.

14. The nucleic acid molecule according to any one of claims 4 to 13, wherein the nucleic acid sequence encoding MEF2C is 5′ relative to a nucleic acid sequence encoding MEF2D and a nucleic acid sequence encoding RXRG is 3′ relative to the nucleic acid sequence encoding MEF2D.

15. The nucleic acid molecule according to any preceding claim, wherein the promoter is selected from the group consisting of Glial fibrillary acidic protein (GFAP), CAR2, CD44, GLUL, PDGFRA, retinaldehyde-binding protein 1 (RLBP1), S100B, SLC1A3, VIM, ProB2, GLAST, CAG and CMV.

16. The nucleic acid molecule according to any preceding claim, wherein the promoter is selected from the group consisting of GFAP, RLBP1, ProB2 and GLAST.

17. The nucleic acid molecule according to any preceding claim, wherein the promoter is a macroglia specific promoter.

18. The nucleic acid molecule according to any preceding claim, wherein the macroglia are retinal macroglia.

19. The nucleic acid molecule according to any preceding claim, wherein the macroglia are Müller glia and/or astrocytes.

20. The nucleic acid molecule according to any preceding claim, wherein the promoter is a GFAP promoter.

21. The nucleic acid molecule according to any preceding claim, wherein the promoter is a gfaABC1D GFAP promoter.

22. A vector comprising the nucleic acid molecule according to any preceding claim.

23. The vector according to claim 22 wherein the vector is a viral vector.

24. The vector according to claim 23 wherein the viral vector is selected from the group consisting of a lentiviral vector, a Sendai vector, a Herpes simplex virus (HSV) vector, an Adenoviral vector, an adeno-associated virus (AAV) vector, an episomal vector and a retroviral vector.

25. The vector according to claim 24 wherein the viral vector is selected from the group consisting of a lentiviral vector, an Adenoviral vector and an adeno-associated virus (AAV) vector.

26. The vector according to claim 25 wherein the viral vector is an AAV vector.

27. The vector according to claim 25 wherein the viral vector is a lentiviral vector.

28. The vector according to claim 22 wherein the vector is a non-viral vector.

29. The vector according to claim 28 wherein the non-viral vector is selected from the group consisting of a liposome, nanoparticle, naked DNA, plasmid and a transposon.

30. The vector according to claim 29 wherein the non-viral vector is a repRNA vector or mRNA.

31. A composition comprising the nucleic acid molecule according to any one of claims 1 to 21 or the vector according to any one of claims 22 to 30, and a pharmaceutically acceptable carrier.

32. A product comprising
(a) a first nucleic acid molecule, according to any one of claims 1 to 21, and
(b) a second nucleic acid molecule, comprising a promoter operably linked to a nucleic acid sequence encoding one or more transcription factor selected from the group consisting of Myocyte Enhancer Factor 2D (MEF2D), Retinoid X Receptor Gamma (RXRG) and Cone-Rod Homeobox (CRX), or functional variants thereof, or any combination thereof, wherein the promoter is for expression of the one or more transcription factor in macroglia;
as a combined preparation for simultaneous, separate or sequential use in the treatment of retinal disease or degeneration.

33. The product according to claim 32 wherein at least one of the first nucleic acid molecule and the second nucleic acid molecule encodes MEF2D or a functional variant thereof and at least one of the first nucleic acid molecule and the second nucleic acid molecule encodes RXRG or a functional variant thereof.

34. The product according to any one of claims 32 to 33 wherein at least one of the first nucleic acid molecule and the second nucleic acid molecule encodes CRX or a functional variant thereof.

35. The product according to any one of claims 32 to 34 wherein
(a) the first nucleic acid molecule comprises a nucleic acid sequence encoding MEF2C and a nucleic acid sequence encoding RXRG, and/or
(b) the second nucleic acid molecule comprises a nucleic acid sequence encoding MEF2D and a nucleic acid sequence encoding CRX.

36. A method of converting a retinal source cell to a retinal target cell by introducing one or more transcription factor comprising MEF2C, or a functional variant thereof, into the retinal source cell, thereby converting the retinal source cell into the retinal target cell.

37. The method of claim 36 wherein the retinal source cell is a macroglia.

38. The method of any one of claims 36 to 37, wherein the retinal source cell is a Müller glia cell or astrocyte.

39. The method of any one of claims 36 to 38, wherein the retinal target cell is a photoreceptor-like cell or a retinal pigment epithelium (RPE)-like cell.

40. The method of claim 39 wherein the photoreceptor-like cell is a cone photoreceptor-like cell.

41. The method of any one of claims 36 to 40, wherein the one or more transcription factor is introduced via a nucleic acid molecule according to any one of claims 1 to 21, a vector according to any one of claims 22 to 30, a composition according to claim 31 or a product according to any one of claims 32 to 35.

42. The method of any one of claims 36 to 41, comprising culturing under suitable conditions for at least 4 days.

43. A cell produced by the method of any one of claims 36 to 42.

44. The nucleic acid molecule according to any one of claims 1 to 21, the vector according to any one of claims 22 to 30, the composition according to claim 31, or the cell according to claim 43 for use in the treatment of retinal disease or degeneration.

45. A method of treating retinal disease or degeneration in a subject comprising administering to a retina of the subject in need thereof a therapeutically effective amount of the nucleic acid molecule according to any one of claims 1 to 21, the vector according to any one of claims 22 to 30, the composition according to claim 31, the product according to any one of claims 32 to 35, or the cell according to claim 43.

46. The nucleic acid molecule according to any one of claims 1 to 21, the vector according to any one of claims 22 to 30, the composition according to claim 31, the product according to any one of claims 32 to 35, the cell according to claim 43 or the method of treating retinal disease or degeneration according to claim 45, wherein the retinal disease or degeneration is age-related macular degeneration (AMD), retinitis pigmentosa (RP), late-stage Best disease, Stargadt macular dystrophy, cone rod dystrophy or glaucoma.

47. The nucleic acid molecule according to any one of claims 1 to 21, the vector according to any one of claims 22 to 30, the composition according to claim 31, the product according to any one of claims 32 to 35, the cell according to claim 43 or the method of treating retinal disease or degeneration according to claim 45 wherein the AMD is dry AMD.

48. The nucleic acid molecule according to any one of claims 1 to 21, the vector according to any one of claims 22 to 30, the composition according to claim 31, the product according to any one of claims 32 to 35, the cell according to claim 43 or the method of treating retinal disease or degeneration according to claim 45 wherein the dry AMD is late-stage dry AMD.
